(19) United States
(12) Patent Application Publication
(10) Pub. No.: US 2023/0331751 A1
(43) Pub. Date: Oct. 19, 2023

(54) STEREOSELECTIVE MANUFACTURE OF SELECTED PURINE PHOSPHORAMIDATES

(71) Applicant: Atea Pharmaceuticals, Inc., Boston, MA (US)

(72) Inventors: Adel Moussa, Burlington, MA (US); Narayan Chaudhuri, Acton, MA (US)

(73) Assignee: Atea Pharmaceuticals, Inc., Boston, MA (US)

(21) Appl. No.: 18/111,316

(22) Filed: Feb. 17, 2023

Related U.S. Application Data
(63) Continuation of application No. PCT/US2021/046778, filed on Aug. 19, 2021.
(60) Provisional application No. 63/129,306, filed on Dec. 22, 2020.
(60) Provisional application No. 63/094,759, filed on Oct. 21, 2020.
(60) Provisional application No. 63/074,302, filed on Sep. 3, 2020.
(60) Provisional application No. 63/067,726, filed on Aug. 19, 2020.

(51) Int. Cl. C07F 9/6561

(52) U.S. Cl. CPC C07F 9/65616; C07B 2200/07

(57) ABSTRACT

The present invention provides stereoselective processes of manufacture for the phosphoramidate nucleotide Compound 1 or a pharmaceutically acceptable salt thereof.

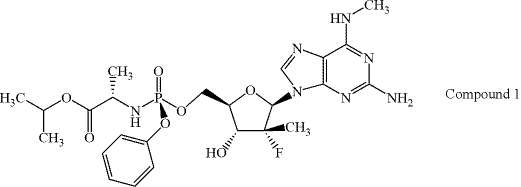 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F]

### CROSS-REFERENCE TO RELATED APPLICATIONS

This application is a continuation of International Patent Application No. PCT/US2021/046778, filed in the U.S. Receiving Office on Aug. 19, 2021, which claims the benefit of U.S. Provisional Application No. 63/067,726, filed Aug. 19, 2020; U.S. Provisional Application No. 63/074,302, filed Sep. 3, 2020; U.S. Provisional Application No. 63/094,759, filed Oct. 21, 2020; and U.S. Provisional Application No. 63/129,306, filed Dec. 22, 2020. The entirety of each of these applications is incorporated by reference for all purposes.

### FIELD OF THE INVENTION

The present invention provides stereoselective processes for the manufacture of purine phosphoramidate nucleotides and intermediates for the production thereof.

### BACKGROUND OF THE INVENTION

Nucleoside analogs have been developed as effective therapeutics for a number of diseases, including cancer, hepatitis C (HCV), hepatitis B (HBV), HIV, and human cytomegalovirus (HCMV). Nucleoside analogs have also been explored for RNA viral infections including viruses of the Flaviviridae family (Dengue Fever, Yellow Fever, Zika Virus), the Filoviridae family (Ebola Virus, Marburg virus), and the Coronaviridae family (SARS-Cov-1 (severe acute respiratory syndrome), SARS-CoV-2 (COVID19) and MERS (Middle East respiratory syndrome coronavirus)).

U.S Pat. Nos. 9,828,410; 10,000,523; 10,005,811; 10,239,911; 10,519,186; 10,815,266; 10,870,672; 10,870,673; 10,875,885 and PCT Applications PCT/US16/21276 (WO2016/144918); PCT/US2017/50323 (WO 2018/048937); PCT/US18/16301 (WO2018/144640); and, PCT/US2019/26837 (WO 2019/200005) disclose Compound 1 or a pharmaceutically acceptable salt of Compound 1, including the hemi-sulfate salt of Compound 1, Compound 1-A, to treat Hepatitis C.

U.S. Pat. No. 10,946,033 and PCT Application PCT/US2017/050323 (WO2018/048937) disclose Compound 1 or a pharmaceutically acceptable salt of Compound 1 to treat certain flaviviruses, including Dengue fever, West Nile fever, Yellow fever, and Zika virus.

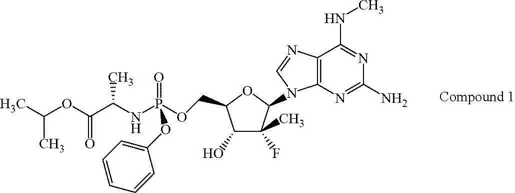 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F]

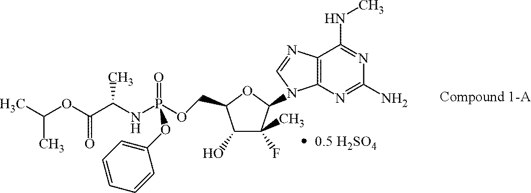

PCT/US21/19468 and U.S. Pat. 10,874,687 describe the use of Compound 1 and Compound 1-A to treat SARS-CoV-2 (COVID-19).

Given the importance of Compound 1 and Compound 1-A for the therapeutic treatment of humans infected with viruses such as a flavivirus, hepatitis C or SARS-CoV-2, it would be useful to provide an advantageous process for manufacture that is scalable.

### SUMMARY OF THE INVENTION

The present invention provides an advantageous and facile stereoselective process for the scalable manufacture of the purine phosphoramidate nucleotide Compound 1 wherein the Sₚ-diastereomer (i.e., the S-stereoconfiguration at the chiral phosphorus atom) is in substantially pure form, e.g., in substantial excess over the Rₚ-diastereomer:

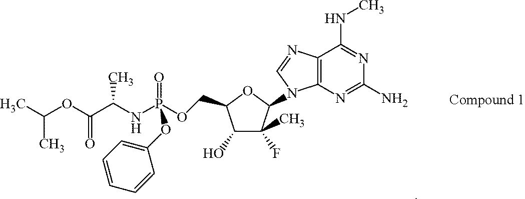

A substantially pure form of the diastereomer typically refers to about 90% or greater of the Sₚ- diastereomer over the Rₚ-diastereomer. In one embodiment, the substantially pure form is about 93% pure or greater, about 95% pure or greater, about 98% pure or greater, or about 99% pure or greater, or even 100% pure. In an alternative embodiment, the substantially pure form is about 80% or greater, about 85% or greater, or about 88% or greater.

The phosphorus S-stereochemistry is set during the reaction of the nucleoside with the phosphoramidate according to the invention. The manufacture of purine Compound 1 according to the present invention includes a coupling reaction of a dihydroquinine salt of a phosphoramidic acid with the requisite purine nucleoside in the presence of a specified activator and a base as described herein.

In a nonlimiting illustrative example of the invention, the process for synthesizing the diastereomerically pure Sₚ-phosphoramidate nucleotide of Compound 1 includes the steps of:
(a) contacting nucleoside Compound 2 with the dihydroquinine salt of isopropyl (hydroxy(phenoxy)phosphoryl)-L-alaninate in the presence of a benzotriazole- or uronium-based activator or other as described herein, such as HATU or COMU, and a base to afford diastereomerically enriched Sₚ-phosphoramidate nucleotide Compound 1 wherein the Sₚ-diastereomer is in substantial excess over the Rₚ-diastereomer:

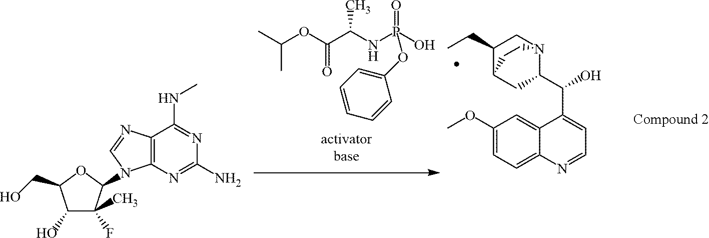

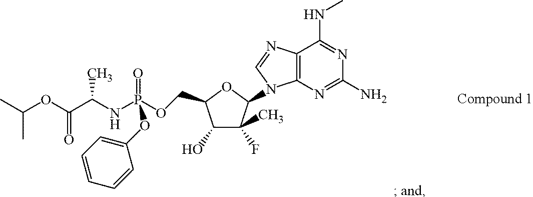

(b) further optionally purifying, e.g., by selective crystallization, the diastereomerically enriched Sₚ-phosphoramidate nucleotide Compound 1 to afford the diastereomerically pure Sₚ-Compound 1 wherein the diastereomerically purity is greater than about 90%, or even greater than about 95% or even about 99% or greater.

In certain embodiments quinine as a salt or freebase can be used for the preparation for dihydroquinine. In one embodiment quinine hemisulfate monohydrate is used for the preparation of dihydroquinine.

In one embodiment, the activator is COMU ((1-cyano-2-ethoxy-2-oxoethylidenaminooxy)dimethylamino-morpholino-carbenium hexafluorophosphate). For the present process invention, COMU is an advantageous activator because it has low shock sensitivity. It has been found in the present invention that the use of COMU as an activator in combination with the dihydroquinine salt of a phosphoramidate and base provides Compound 1 in a high isolated yield. Additionally, use of the COMU activator can result in the preparation of Compound 1 with high diastereoselectivity. Use of COMU as the activator can also allow the reaction to proceed efficiently and/or at relatively low reaction temperature.

Alternatively, the process can be carried out using an activator such as a benzotriazole-based activator, including, but not limited to HOBt ((1-hydroxybenzotriazole), PyBOP (benzotriazol-1-yloxytri(pyrrolidino)phosphonium hexafluorophosphate), HATU (O-(7-azabenzotriazol-1-yl)-1,1,3,3-tetramethyluronium hexafluorophosphate), HBTU (3-[bis(dimethylamino)methyliumyl]-3H-benzotriazol-1-oxide hexafluorophosphate), HCTU (2-(6-chloro-1H-benzotriazole-1-yl)-1,1,3,3-tetramethylaminium hexafluorophosphate), or TBTU (O-benzotriazol-1-yl-1,1,3,3-tetramethyluronium tetrafluoroborate). In one embodiment, the activator is HATU.

Examples 9, 10, and 12 below provide nonlimiting illustrations of the process of manufacture of Compound 1 that includes the activator HATU, which can provide the target Compound 1 in high yield. Any impurities and byproducts associated with use of HATU can be removed by washing and selective crystallization of Compound 1. Use of HATU as the activator in the present invention can provide Compound 1 in high yield.

Example 13 illustrates the process of manufacture of Compound 1 that includes the activator COMU, which can also provide the target Compound 1 in high yield. Likewise, any impurities and byproducts associated with use of COMU can be removed by washing and selective crystallization of Compound 1. As with HATU, use of COMU as the activator in the present invention can provide Compound 1 in high yields.

In one embodiment, the base used in the coupling reaction is selected from NR₃ wherein R can be selected independently in each instance from H, alkyl, aryl, heteroaryl, alkenyl, alkynyl, benzyl and allyl, and wherein NR₃ typically has at least one, and often two or three, non-hydrogen R groups. In one embodiment, the base is DIPEA (N,N-diisopropylethylamine) or NEt₃ (triethylamine). Other nonlimiting examples of the base are alkyl substituted amines generally, or DABCO (1,4-diazabicyclo[2.2.2]octane), DBU (1,8-diazabicyclo[5.4.0]undec-7-ene), N-methyl morpholine, diethylamine or monoethylamine, In an alternative embodiment, the base is quinine or quinidine. In one embodiment, the base is quinine.

It has been surprisingly discovered that the dihydroquinine salt assists in directing the stereochemistry of Compound 1 to the Sₚ-diastereomer during the coupling reaction. The tertiary amine base in coupling reactions is generally considered a spectator to the bond-forming event. The present invention instead demonstrates the unexpected result that the dihydroquinine base not only participates, but directs, the bond forming step. This discovery has been used to develop the present process that delivers Compound 1 in a substantially diastereomerically enriched form.

Additional purification of the Sₚ-diastereomer can be obtained, for example: (i) by selective crystallization in a solvent or solvent/anti-solvent system, as described in more detail below; (ii) trituration of an anti-solvent into a solvent-based solution of Compound 1, or (iii) any method known to skilled chemists that results in such purification, including column chromatography, etc. Exemplary details of the crystallization procedure are provided below. Nonlimiting examples of the crystallization solvent are polar organic solvents such as an alkyl ester, for example ethyl acetate or isopropyl acetate, acetonitrile, DMSO, methylene chloride, acetone, or the like. Nonlimiting examples of suitable anti-solvents are non-polar organic liquids such as hydrocarbons that can be removed from the final product, including but not limited to pentane, hexane, heptane, or the like.

Importantly, in certain embodiments, this manufacturing process may be accomplished without a required extra step of protecting the N⁶-methyl, N²-amino-2,6-diaminopurine base during the reaction, which is advantageous for the efficiency of the full process. In a principal embodiment, neither the amine of the N⁶-methyl or the N²-amino in the diaminopurine is protected or substantially derivatized during the process. This embodiment minimizes the need for tangential protection and/or deprotection steps.

In one embodiment, step (a) is conducted in a polar aprotic solvent, for example dimethylformamide (DMF), dichloromethane (DCM), tetrahydrofuran (THF), 2-methyltetrahydrofuran (2-MeTHF), ethyl acetate (EtOAc), acetonitrile (MeCN), dimethyl sulfoxide (DMSO), acetone, or N-methylpyrrolidone. In one embodiment, step (a) is carried out in dichloromethane (DCM). In one embodiment, step (a) is carried out in 2-methyltetrahydrofuran (2-MeTHF). In some embodiments, step (a) is carried out in a mixture of solvents. In one embodiment, step (a) is carried out in a mixture of dichloromethane (DCM) and 2-methyltetrahydrofuran (2-MeTHF).

In one aspect of the present invention, the manufacture of Compound 2 comprises the steps of:

(1.2.a) the protection of the 5′ hydroxyl and the 3′ hydroxyl on the nucleoside (3S,4R,5R)-3,4-dihydroxy-5-(hydroxymethyl)-3-methyldihydrofuran-2(3H)-one to afford a compound of Formula I wherein R^{1ª} and R^{1b} are oxygen protecting groups and at least one of R^{1ª} and R^{1b} is a carbonate such as —C(O)OC₁-₆alkyl (for example, —C(O)OCH₃ or —C(O)OtBu), —C(O)O-benzyl, or —CH₂-phenyl wherein the phenyl group is substituted with at least one substituent selected from alkoxy (including but not limited to methoxy and ethoxy), hydroxy, nitro, bromo, chloro, fluoro, azido, and haloalkyl, or in an alternative embodiment, at least one of R^{1ª} and R^{1b} are —C(O)OC₁₋₂₀alkyl, —C(O)OC₂₋₂₀alkenyl, or —C(O)NR^{10a}R^{10b} wherein R^{10a} and R^{10b} are independently selected from hydrogen, C₁₋₂₀alkyl, C₂₋₂₀alkenyl, aryl, arylalkyl, heteroaryl, and heteroarylalkyl wherein the C₁₋₂₀alkyl, C₂-₂₀alkenyl, aryl, arylalkyl, heteroaryl, and heteroarylalkyl can optionally be substituted with at least one substituent selected from alkoxy (including but not limited to methoxy and ethoxy), hydroxy, nitro, bromo, chloro, fluoro, azido, and haloalkyl:

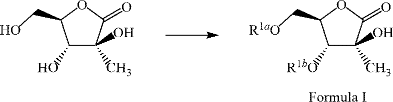

(1.2.b) the conversion of the alcohol of Formula I into a monofluoride with inversion of stereochemistry to afford a compound of Formula II:

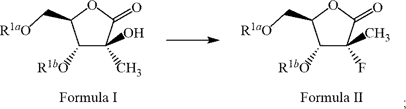

(1.2.c) the reduction of the lactone of the nucleoside compound of Formula II to afford the nucleoside compound of Formula III:

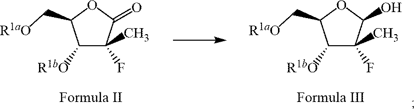

(1.2.d) the conversion of a compound of Formula III to a compound of Formula IV wherein X is Cl, Br, or OAc:

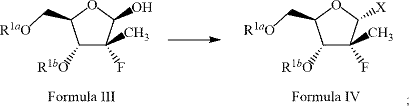

(1.2.e) the nucleophilic substitution of the compound of Formula IV with 2-amino-6-chloropurine to afford a compound of Formula V:

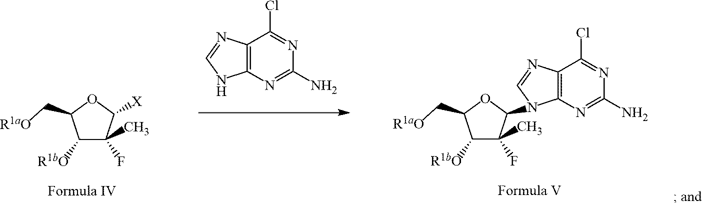

(1.2.f) the conversion of the 2-amino-6-chloropurine base to the 2-amino-N⁶-methyl base and the deprotection of the 3′ and 5′-positions to afford Compound 2:

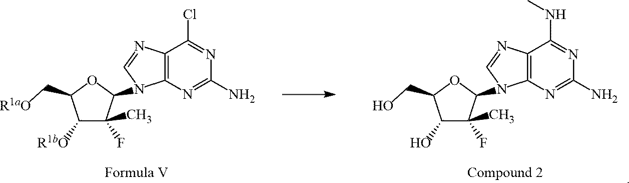

In one embodiment, Compound 2 is prepared via the process below wherein R^{1a} and R^{1b} are —C(O)O—^{t}Bu or a “Boc” group:

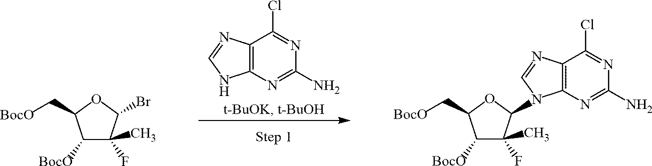

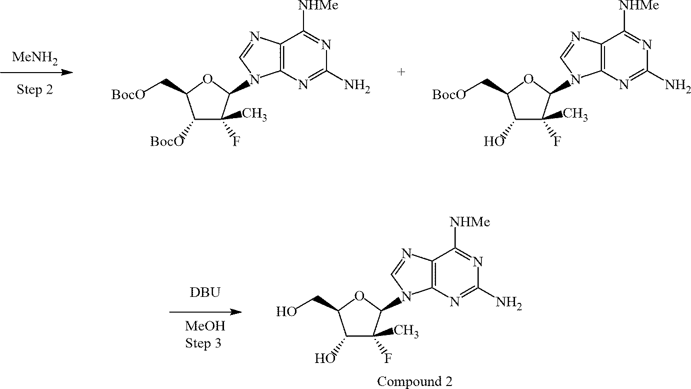

The dicarbonate product of Step 1 above can be carried forward without purification. Alternatively, in one aspect of the invention, demonstrated in Example 13, the product of Step 1 can be purified by selective crystallization. Crystallization of the product in a mixture of DCM and n-heptane provides a pure compound that can be used in the next step to reduce the number of impurities.

Another aspect of the present invention is a novel dicarbonate intermediate in step 1, illustrated below, which can optionally be used in the crystalline form. Isolation of the pure compound in crystalline form provides an additional opportunity to control impurities and monitor the present process.

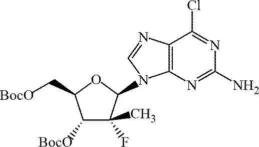

Further, the following dicarbonate intermediate is also a novel intermediate compound:

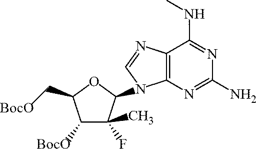

The product mixture of Step 2 can also be purified by selective crystallization of the dicarbonate product, providing yet another opportunity for control and monitoring. Alternatively, in some aspects of the present invention, the mixture of products from Step 2 are taken into Step 3 without isolation. It has been found that doing so does not affect the yield or purity of the final Compound 2.

An additional aspect of the invention describes deprotection of the Boc groups under basic conditions to afford Compound 2. Performing the deprotection under basic conditions allows the product to be ready for coupling to the phosphoramidate prodrug following selective crystallization for purification. Using acidic conditions that are more typically used to deprotect Boc groups, there can be a need to have an additional step to neutralize the salt and purification in order to prepare Compound 2 for installation of the phosphoramidate prodrug.

The diastereomerically enriched phosphoramidate Compound 1 prepared by the present process can be a mixture of Sₚ:Rₚ diastereomers wherein the Sₚ diastereomer is in excess of the Rₚ diastereomer. In one embodiment, the ratio of Sₚ:Rₚ diastereomers in a diastereomerically enriched phosphoramidate Compound 1 is greater than about 51:49, greater than about 55:45, greater than about 60:40, greater than about 65:35, greater than about 70:30, greater than about 75:25, greater than about 80:20, greater than about 85:15, greater than about 90:10, greater than about 95:5, greater than about 98:2, or greater than about 99:1.

In certain embodiments, the process can include a purification step of the enriched mixture of Compound 1. One nonlimiting example is selective crystallization of the enriched mixture in an appropriate solvent. Nonlimiting examples are an alkyl acetate solvent such as ethyl acetate or isopropyl acetate, a chlorinated solvent, such a dichloromethane, a ketone solvent, such as acetone, an aromatic solvent, such as toluene, or a mixture thereof. In some embodiments, the purification is conducted via selective crystallization in an alkyl acetate solvent, for example isopropyl acetate. In an embodiment, the purification is conducted via selective crystallization from a solvent, for example, an alkyl acetate, a chlorinated solvent, a ketone solvent, or a mixture thereof, with an anti-solvent, for example, acetonitrile or an aliphatic hydrocarbon. In one embodiment, the purification is conducted via selective crystallization from a mixture of ethyl acetate and toluene.

In certain aspects of the invention, Compound 1 is prepared as a pharmaceutically acceptable salt, for example, by reaction with a pharmaceutically acceptable acid, as described more fully herein.

In one embodiment, the pharmaceutically acceptable salt form of Compound 1 is the hemisulfate salt form, Compound 1-A:

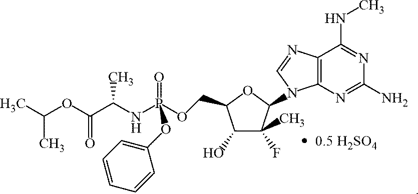

In an aspect of the present invention, the manufacture of the dihydroquinine salt of isopropyl (hydroxy(phenoxy)phosphoryl)-L-alaninate comprises the steps of:
(1.1.a) the coupling of phenyl dichlorophosphate with benzyl alcohol to generate the benzyl phenyl phosphorochloridate in situ that is subsequently reacted with L-alanine isopropyl ester hydrochloride to afford isopropyl ((benzyloxy)(phenoxy)phosphoryl)-L-alaninate:

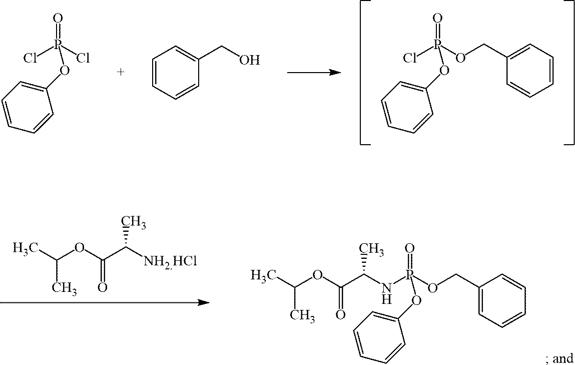

(1.1.b) debenzylation of isopropyl ((benzyloxy)(phenoxy)phosphoryl)-L-alaninate and in situ reduction of quinine to afford the dihydroquinine salt of isopropyl (hydroxy(phenoxy)phosphoryl)-L-alaninate:

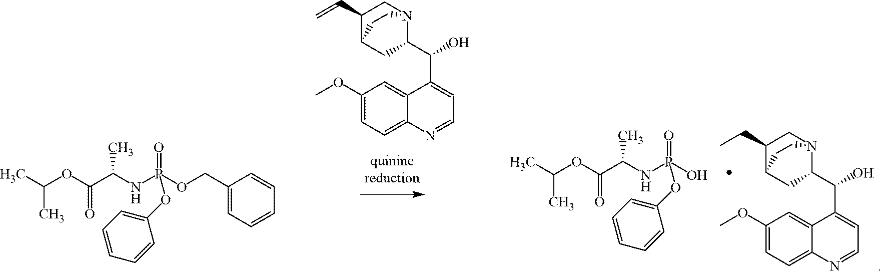

In an alternative embodiment of (1.1.b), quinine is reduced to dihydroquinine in a separate reaction.

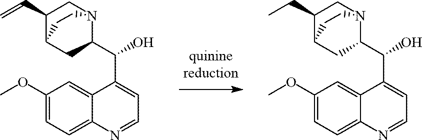

In certain embodiments quinine freebase can be used for the preparation for dihydroquinine. In one embodiment quinine hemisulfate monohydrate is used for the preparation of dihydroquinine.

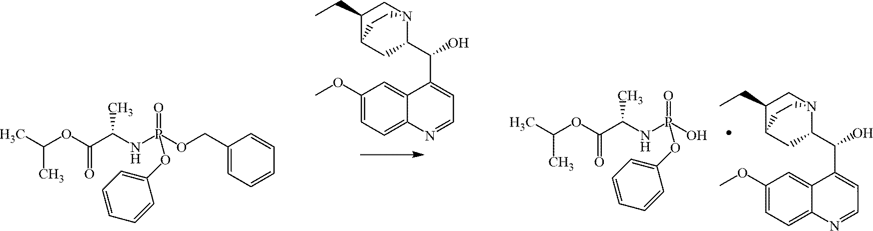

The independently reduced dihydroquinine can then be added to the debenzylation reaction, optionally following purification. While adding an additional step, it has been found that this method produces the dihydroquinine salt of isopropyl (hydroxy(phenoxy)phosphoryl)-L-alaninate in high yields and with high purity.

In one embodiment, Compound 2 is prepared via the process below wherein R^{1a} and R^{1b} are —C(O)O-benzyl or a “carboxybenzyl” (Cbz) group:

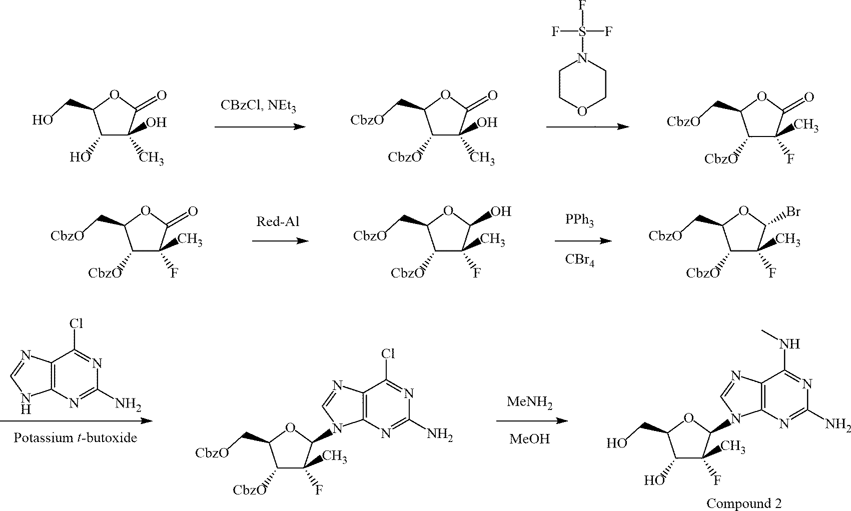

In an alternative embodiment, Compound 2 is prepared via the process below wherein R^{1a} and R^{1b} are —C(O)OCH₃:

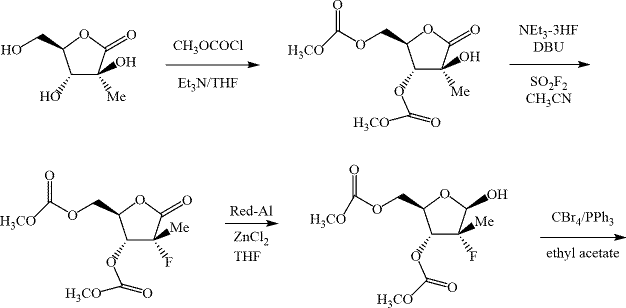

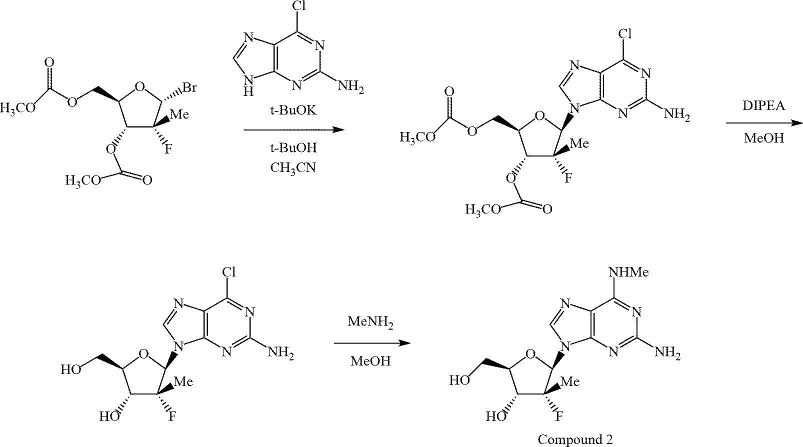

In a further alternative embodiment, the manufacture of Compound 2 comprises the steps of:
(1.2.a) the protection of nucleoside (3S,4R,5R)-3,4-dihydroxy-5-(hydroxymethyl)-3-methyldihydrofuran-2(3H)-one at the 3′- and 5′-hydroxyl groups wherein the protecting group links together the 3′ - and 5′-hydroxyl groups to form a bridge structure:

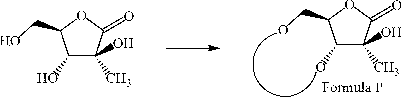

and the bridge structure is selected from

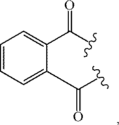

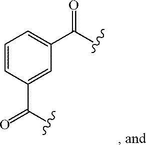

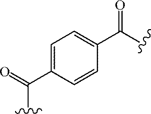

wherein the phenyl group of the bridged structure can be substituted with substituents selected from alkoxy (including but not limited to methoxy and ethoxy), hydroxy, nitro, bromo, chloro, fluoro, azido, and haloalkyl;
(1.2.b) the conversion of the alcohol of Formula I′ into a monofluoride with inversion of stereochemistry to afford a compound of Formula II′:

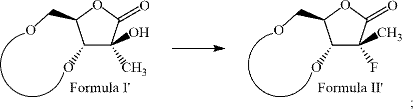

(1.2.c) the reduction of the lactone of the nucleoside compound of Formula II′ to afford the nucleoside compound of Formula III′:

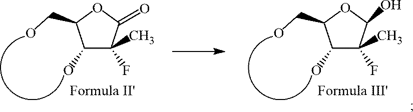

(1.2.d) the conversion of a compound of Formula III′ to a compound of Formula IV′ wherein X is Cl, Br, or OAc:

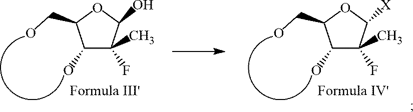

(1.2.e) the nucleophilic substitution of the compound of Formula IV′ with 2-amino-6-chloropurine to afford a compound of Formula V′:

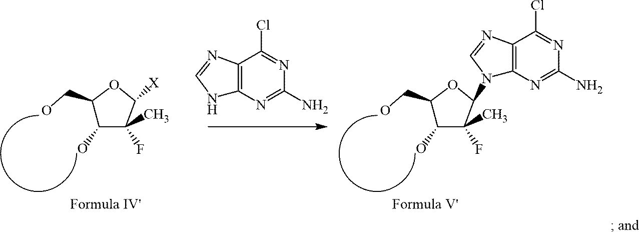

(1.2.f) the conversion of the 2-amino-6-chloropurine base to the 2-amino-N⁶-methyl base and the deprotection of the 3′ and 5′-positions to afford Compound 2:

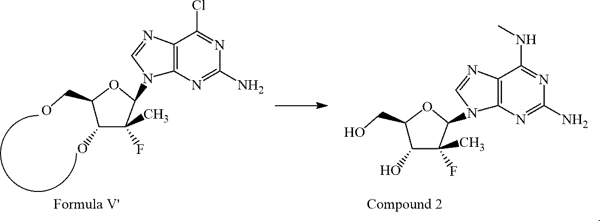

In an alternative embodiment, Compound 2 can be prepared via the process below:

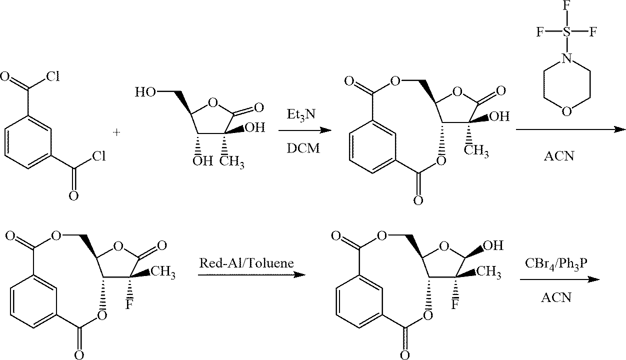

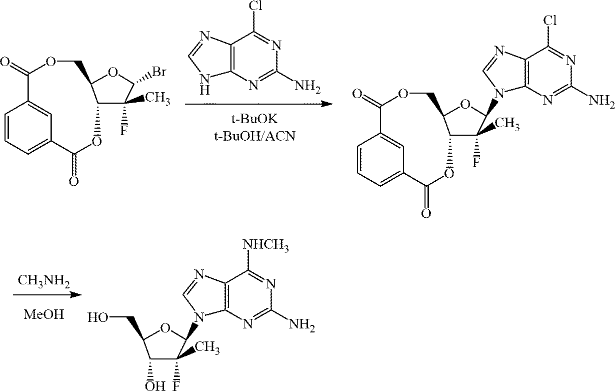

In one embodiment, Compound 1 and Compound 1-A are prepared via the process below using Compound 2:

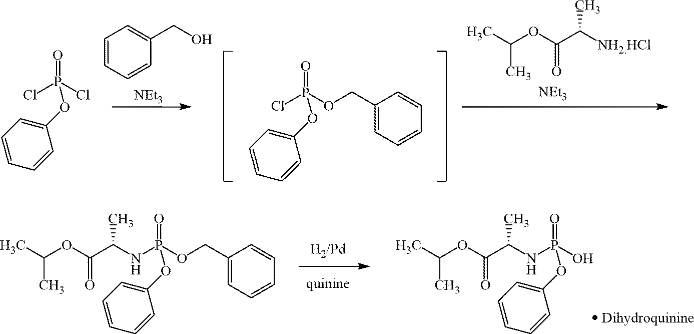

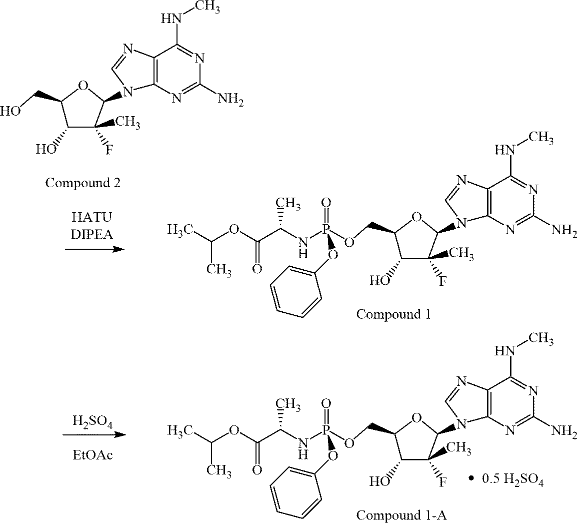

In an alternative embodiment, Compound 1 and Compound 1-A are prepared via the process below using Compound 2:

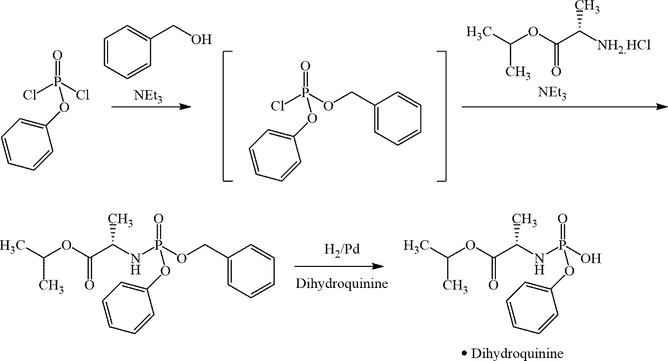

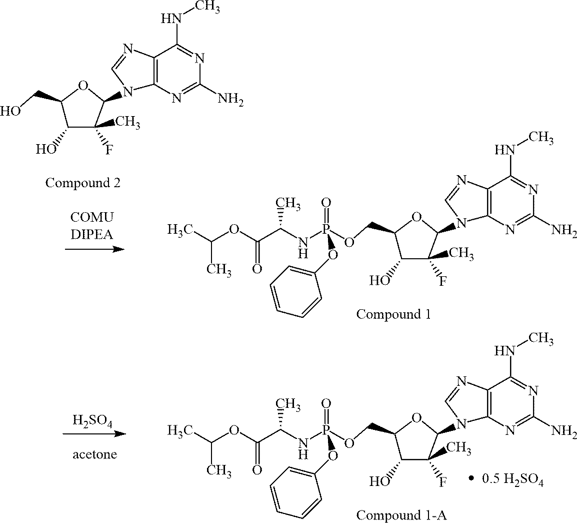

In an alternative embodiment, the coupling of Compound 2 with the dihydroquinine salt of isopropyl (hydroxy(phenoxy)phosphoryl)-L-alaninate is conducted in the presence of the benzotriazole-based activator HATU and the base quinine:

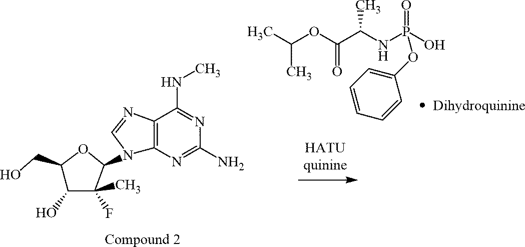

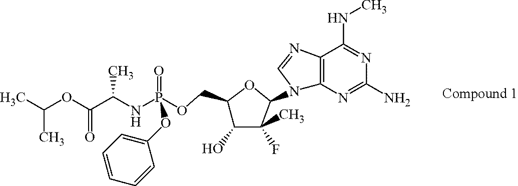 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@@]1(C)F]

In an alternative embodiment, the coupling of Compound 2 with the dihydroquinine salt of isopropyl (hydroxy(phenoxy)phosphoryl)-L-alaninate is conducted in the presence of COMU as activator and the base quinine:

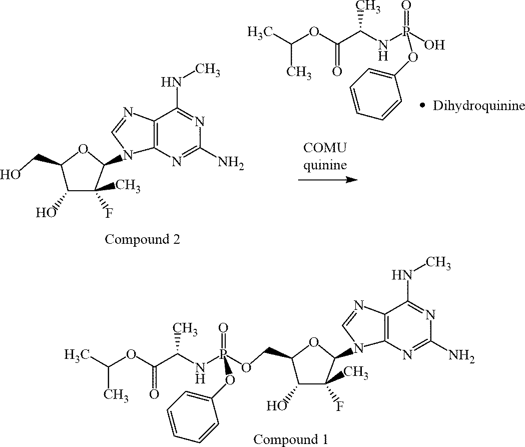

In another aspect, the present invention provides the dihydroquinine salt of isopropyl (hydroxy(phenoxy)phosphoryl)-L-alaninate:

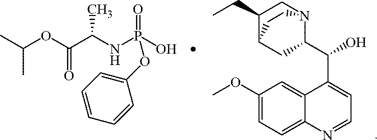

The dihydroquinine salt of isopropyl (hydroxy(phenoxy)phosphoryl)-L-alaninate gives the diastereoselectivity to the coupling reaction with activator and base. Despite starting with a racemic phosphorus atom in the prodrug precursor, use of a chiral tertiary amine produces a substantially Sₚ-enriched product. This unexpected effect makes the dihydroquinine salt of isopropyl (hydroxy(phenoxy)phosphoryl)-L-alaninate advantageous to the production of Compound 1.

In another aspect, the present invention also provides carbonate or carbamate compounds of Formula IIA and Formula IIIA as well as bridged compounds of Formula II′ and Formula III′:

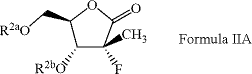

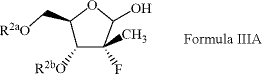

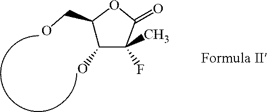

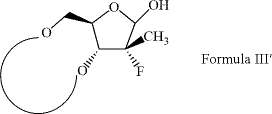

or a pharmaceutically acceptable salt thereof wherein
R^{2a} and R^{2b} are oxygen protecting groups and at least one of R^{2a} and R^{2b} is —C(O)OC₁₋₆alkyl (for example —C(O)OtBu or —C(O)OCH₃) or —C(O)O-benzyl or in an alternative embodiment, at least one of R^{2a} and R^{2b} is —C(O)OC₁₋₂₀alkyl, —C(O)OC₂₋₂₀alkenyl, or —C(O)NR^{10a}R^{10b} wherein R^{10a} and R^{10b} are independently selected from hydrogen, C₁₋₂₀alkyl, C₂₋₂₀alkenyl, aryl, arylalkyl, heteroaryl, and heteroarylalkyl wherein R^{2a} and R^{2b} can independently be optionally substituted with a substituent selected from alkoxy, hydroxy, nitro, bromo, chloro, fluoro, azido, and haloalkyl; and
wherein the bridge structure in Formula II′ and Formula III′ is selected from:

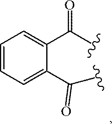

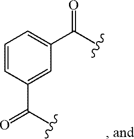

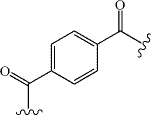

wherein the phenyl group of the bridge structure can be substituted with substituents selected from alkoxy (including but not limited to methoxy and ethoxy), hydroxy, nitro, bromo, chloro, fluoro, azido, and haloalkyl.

In one embodiment, R^{2a} and R^{2b} are both —C(O)OC₁₋₆alkyl, for example —C(O)OtBu. In one embodiment, R^{2a} and R^{2b} are both —C(O)O-benzyl. In one embodiment, R^{2a} is —C(O)OC₁₋₆alkyl or —C(O)O-benzyl and R^{2b} is an oxygen protecting group which when attached to the oxygen is an ester, ether, or silyl ether moiety. In an alternative embodiment, R^{2b} is —C(O)OC₁₋₆alkyl or -C(O)O-benzyl and R^{2a} is an oxygen protecting group which when attached to the oxygen is an ester, ether, or silyl ether moiety.

In an alternative embodiment, R^{2a} and R^{2b} are both —C(O)OCH₃. In one embodiment, R^{2a} is —C(O)OCH₃ and R^{2b} is an oxygen protecting group which when attached to the oxygen is an ester, ether, or silyl ether moiety. In an alternative embodiment, R^{2b} is —C(O)OCH₃ and R^{2a} is an oxygen protecting group which when attached to the oxygen is an ester, ether, or silyl ether moiety.

In an alternative embodiment, R^{2a} and R^{2b} are both —C(O)OC₁₋₂₀alkyl, for example —C(O)OC₁₋₆alkyl, —C(O)OC₇₋₁₀alkyl, —C(O)OC₁₁₋₁₄alkyl, —C(O)OC₁₅₋₁₇alkyl, or —C(O)OC₁₈₋₂₀alkyl. In one embodiment, R^{2a} and R^{2b} are both —C(O)OC₁₆H₃₃. In one embodiment, R^{2a} is —C(O)OC₁₆H₃₃ and R^{2b} is an oxygen protecting group which when attached to the oxygen is an ester, ether, or silyl ether moiety. In an alternative embodiment, R^{2b} is —C(O)OC₁₆H₃₃ and R^{2a}is an oxygen protecting group which when attached to the oxygen is an ester, ether, or silyl ether moiety.

In an alternative embodiment, R^{2a} and R^{2b} are both —C(O)OC₂₋₂₀alkenyl, for example —C(O)OC₂₋₆alkenyl —C(O)OC₆₋₁₀alkenyl, —C(O)OC₁₀₋₁₄alkenyl, —C(O)OC₁₄₋₁₈alkenyl, or —C(O)OC₁₈₋₂₀alkenyl. In one embodiment, R^{2a} and R^{2b} are both —C(O)OC₂₋₂₀alkenyl. In one embodiment, R^{2a} is —C(O)OC₂₋₂₀alkenyl and R^{2b} is an oxygen protecting group which when attached to the oxygen is an ester, ether, or silyl ether moiety. In an alternative embodiment, R^{2b} is —C(O)OC₂₋₂₀alkenyl and R^{2a} is an oxygen protecting group which when attached to the oxygen is an ester, ether, or silyl ether moiety.

In an alternative embodiment, R²^{a} and R^{2b} are both —C(O)NR^{10a}R^{10b}, for example —C(O)NHPh, —C(O)NHBn, —C(O)N(Ph)₂, —C(O)N(Bn)₂, —C(O)NHC₁₋₂₀alkyl (including, but not limited to, —C(O)NHCH₃, —C(O)NHtBu, and —C(O)NHC₁₆H₃₃), and —C(O)N(C₁₋₂₀alkyl)₂ including, but not limited to, —C(O)N(CH₃)₂, —C(O)N(tBu)₂, and —C(O)N(C₁₆H₃₃)₂). In one embodiment, R^{2a} and R^{2b} are both —C(O)NR^{10a}R^{10b}. In one embodiment, R^{2a} is —C(O)NR^{10a}R^{10b} and R^{2b} is an oxygen protecting group which when attached to the oxygen is an ester, ether, or silyl ether moiety. In an alternative embodiment, R^{2b} is —C(O)NR^{10a}R^{10b} and R^{2a} is an oxygen protecting group which when attached to the oxygen is an ester, ether, or silyl ether moiety.

### BRIEF DESCRIPTION OF THE FIGURES

FIG. 1 is a diffractogram collected for the sample of Compound 1-A obtained from Experiment 1 in Example 13. The X-axis is measured in degrees, while the Y-axis shows intensity in counts. Details are provided in Table 3 of Example 14.

FIG. 2 is a diffractogram collected for the sample of Compound 1-A obtained from Experiment 2 in Example 13. The X-axis is measured in degrees, while the Y-axis shows intensity in counts. Details are provided in Table 4 of Example 14.

FIG. 3 is a diffractogram collected for the sample of Compound 1-A obtained from Experiment 3 in Example 13. The X-axis is measured in degrees, while the Y-axis shows intensity in counts. Details are provided in Table 5 of Example 14.

FIG. 4 is a the thermogram collected for the sample of Compound 1-A obtained from Experiment 1 in Example 13. The X-axis is measured in degrees Celsius, while the Y-axis displays heat flow in milliwatts. Details are provided in Example 15.

FIG. 5 is a the thermogram collected for the sample of Compound 1-A obtained from Experiment 2 in Example 13. The X-axis is measured in degrees Celsius, while the Y-axis displays heat flow in milliwatts. Details are provided in Example 15.

FIG. 6 is a thermogram collected for the sample of Compound 1-A obtained from Experiment 3 in Example 13. The X-axis is measured in degrees Celsius, while the Y-axis displays heat flow in milliwatts. Details are provided in Example 15.

FIG. 7 is a scheme of the reaction to form compound 1 from compound 2 and the dihydroquinine salt of isopropyl (hydroxy(phenoxy)phosphoryl)-L-alaninate in the presence of an activator and base.

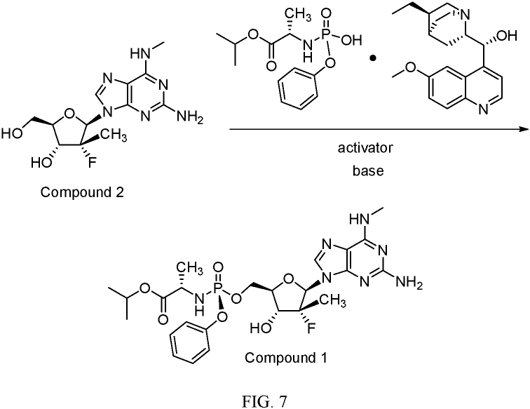

### DETAILED DESCRIPTION OF THE INVENTION

The present invention provides stereoselective processes for the manufacture of the purine phosphoramidate nucleotide Compound 1 wherein the Sₚ-diastereomer is in a substantially pure form, e.g., in excess over the Rₚ-diastereomer:

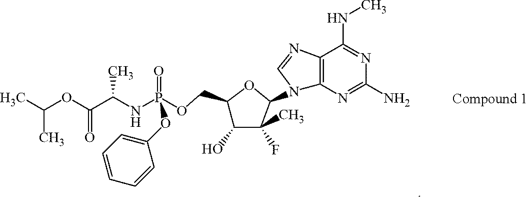

A substantially pure form of the diastereomer refers to about 90% or greater of the Sₚ-diastereomer over the Rₚ-diastereomer. In one embodiment, the substantially pure form is about 93% pure or greater, about 95% pure or greater, about 98% pure or greater, about 99% pure or greater, or even 100% pure. In an alternative embodiment, the substantially pure form is about 80% or greater, about 83% or greater, about 85% or greater, or about 88% or greater.

In one embodiment, Compound 1 is prepared as a pharmaceutically acceptable salt, for example, by reaction with a pharmaceutically acceptable acid, as described more fully herein.

In one embodiment, the pharmaceutically acceptable salt form of Compound 1 is the hemisulfate salt form, Compound 1-A:

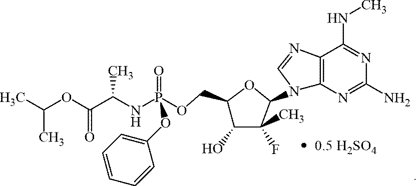

In one embodiment, Compound 1-A is prepared from Compound 1 by the dropwise addition of concentrated H₂SO₄ in EtOAc or MeOH and the filtration of the resulting precipitate. In an alternative embodiment, Compound 1-A is prepared from Compound 1 by the dropwise addition of concentrated H₂SO₄ in acetone.

In one aspect of the present invention, the process for synthesizing the diastereomerically pure Sₚ-phosphoramidate nucleotide of Compound 1 comprises the steps of:
(a) contacting the nucleoside compound of Compound 2 with the dihydroquinine salt of isopropyl (hydroxy(phenoxy)phosphoryl)-L-alaninate in the presence of a specified activator as described herein and base described herein to afford diastereomerically enriched Sₚ-phosphoramidate nucleotide Compound 1 wherein the Sₚ-diastereomer is in substantial excess over the Rₚ-diastereomer:

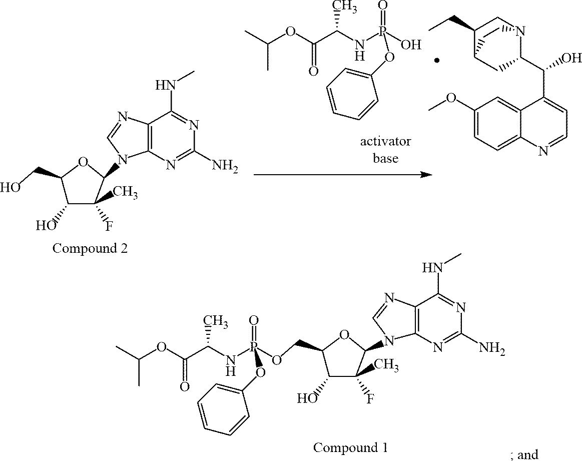

(b) further optionally purifying, e.g., by selective crystallization, the diastereomerically enriched Sₚ-phosphoramidate nucleotide Compound 1 to afford the diastereomerically pure Sₚ-Compound 1 wherein the diastereomerically purity is greater than about 90%, or even greater than about 95% or even about 99% or greater.

An additional optional step includes:

(c) preparing the pharmaceutically acceptable salt form of the diastereomerically pure Sₚ-purine phosphoramidate nucleotide Compound 1.

In one embodiment, the activator is COMU ((1-Cyano-2-ethoxy-2-oxoethylidenaminooxy)dimethylamino-morpholino-carbenium hexafluorophosphate).

In one embodiment, the activator is HATU (O-(7-azabenzotriazol-1-yl)-1,1,3,3-tetramethyluronium hexafluorophosphate).

In some embodiments, an alternative activator is used, typically a benzotriazole-based activator, including, but not limited to HOBt ((1-hydroxybenzotriazole), PyBOP (benzotriazol-1-yloxytri(pyrrolidino)phosphonium hexafluorophosphate), HBTU (3-[bis(dimethylamino)methyliumyl]-3H-benzotriazol-1-oxide hexafluorophosphate), HCTU (2-(6-chloro-1H-benzotriazole-1-yl)-1,1,3,3-tetramethylaminium hexafluorophosphate), and TBTU (O-benzotriazol-1-yl-1,1,3,3-tetramethyluronium tetrafluoroborate).

In one embodiment, the base is selected from NR₃ wherein R can be selected independently in each instance from H, alkyl, aryl, heteroaryl, alkenyl, alkynyl, benzyl and allyl, and which typically has at least one, and often two or more, non-hydrogen R groups. In one embodiment, the base is DIPEA (N,N-diisopropylethylamine). In one embodiment, the base is NEt₃ (triethylamine). In an alternative embodiment, the base is selected from DMAP, (S)-C₅Ph₅-DMA-P, (R)-C₅Me₅-DMAP, quinidine, quinine, TEA, DBU, TMEDA, imidazole, and K₂CO₃. In one embodiment, the base is quinine. In one embodiment, the base is dihydroquinine.

In some embodiments the specified activator is a uronium-type activator selected from HBTU, HATU, COMU, and TFFH and the base is DIPEA. In one embodiment, the activator is COMU and the base is NEt₃. In another embodiment the activator is COMU and the base is DIPEA.

In some embodiments, the specified activator is a benzotriazole-based activator selected from HOBt, PyBOP, HATU, HBTU, HCTU, and TBTU and the base is DIPEA. In one embodiment, the activator is a benzotriazole-based activator selected from HOBt, PyBOP, HATU, HBTU, HCTU, and TBTU and the base is NEt₃. In one embodiment, the activator is HATU and the base is DIPEA. In one embodiment, the activator is HATU and the base is NEt₃.

In some embodiments the specified activator is a uronium-type activator selected from HBTU, HATU, COMU, and TFFH and the base is quinine. In one embodiment, the activator is COMU and the base is quinine. In another embodiment the activator is COMU and the base is dihydroquinine or quinine.

In some embodiments, the specified activator is a benzotriazole-based activator selected from HOBt, PyBOP, HATU, HBTU, HCTU, and TBTU and the base is quinine, or its salt or salt hydrate. In one embodiment, the activator is a benzotriazole-based activator selected from HOBt, PyBOP, HATU, HBTU, HCTU, and TBTU and the base is quinine. In one embodiment, the activator is HATU and the base is dihydroquinine. In one embodiment, the activator is HATU and the base is quinine.

In one embodiment, step (a) is conducted in a polar aprotic solvent, including dimethylformamide (DMF), dichloromethane (DCM), tetrahydrofuran (THF), 2-methyltetrahydrofuran (2-MeTHF), ethyl acetate (EtOAc), acetonitrile (MeCN), dimethyl sulfoxide (DMSO), acetone, and N-methylpyrrolidone. In one embodiment, the solvent of step (a) is DCM. In one embodiment the solvent of step (a) is 2-MeTHF. In some embodiments, the solvent of step (a) is a mixture of solvents. In one embodiment, the solvent of step (a) is a mixture of DCM and 2-MeTHF.

The temperature for the reaction or purification of each step is chosen independently. In some embodiments, step (a), is performed at or below about -20° C. In some embodiments, step (a), is performed performed at or below about 0° C. In some embodiments, step (a), is performed at or below about 10° C. In some embodiments, step (a), is performed between about 10° C. and about 30° C. In some embodiments, step (a), is performed at or above about 30° C. In some embodiments, step (a), is performed at or above about 50° C. In some embodiments, step (a), is performed at or above about 70° C. Step (a) can be run at any temperature that achieves the desired result.

Step (a) affords the diastereomerically enriched phosphoramidate Compound 1 wherein the Sₚ-diastereomer is in excess of the Rₚ-diastereomer. In one embodiment, the ratio of Sₚ:Rₚ diastereomers in the diastereomerically enriched Compound 1 is greater than about 51:49, greater than about 55:45, greater than about 60:40, greater than about 65:35, greater than about 70:30, greater than about 75:25, greater than about 80:20, greater than about 85:15, greater than about 90: 10, greater than about 95:5, greater than about 98:2, or greater than about 99:1.

In one embodiment, the purification in step (b) is the selective crystallization of the enriched mixture, for example, in an alkyl acetate solvent such as ethyl acetate or isopropyl acetate, a chlorinated solvent, such a dichloromethane, a ketone solvent, such as acetone, an aromatic solvent, such as toluene, or a mixture thereof to afford pure a Sₚ-Compound 1. In one embodiment, the purification is conducted via selective crystallization from an alkyl acetate, such as isopropyl acetate. In certain embodiments, the purification is conducted via a selective crystallization from a mixture of ethyl acetate and toluene.

In one embodiment, the purification in step (b) is the selective crystallization of the enriched mixture wherein the enriched mixture is dissolved in an organic solvent and then an anti-solvent is added dropwise to the above solution system wherein the organic solvent comprises a solvent selected from C₁₋₈ alcohols, C₂₋₈ ethers, C₃₋₇ ketones, C₃₋₇ esters, C₁₋₂ chlorocarbons, and C₂₋₇ nitriles and wherein the anti-solvent comprises a solvent selected from C₅₋₁₂ saturated hydrocarbons, C₆₋₁₂ aromatic hydrocarbons, and petroleum ether. In one embodiment, the organic solvent is selected from ethyl acetate, tert-butyl methyl ether, isopropanol or tetrahydrofuran. In one embodiment, the anti-solvent is selected from petroleum ether or hexane.

In one embodiment, diastereomerically pure Compound 1 is greater than about 95% pure, greater than about 96%, greater than about 98%, greater than about 99%, or 100% pure.

In one aspect of the present invention, the synthesis process of the dihydroquinine salt of isopropyl (hydroxy(phenoxy)phosphoryl)-L-alaninate comprises the steps of:
(1.a) the coupling of phenyl dichlorophosphate with benzyl alcohol to generate the benzyl phenyl phosphorochloridate in situ that is subsequently reacted with L-alanine isopropyl ester hydrochloride to afford isopropyl ((benzyloxy)(phenoxy)phosphoryl)-L-alaninate:

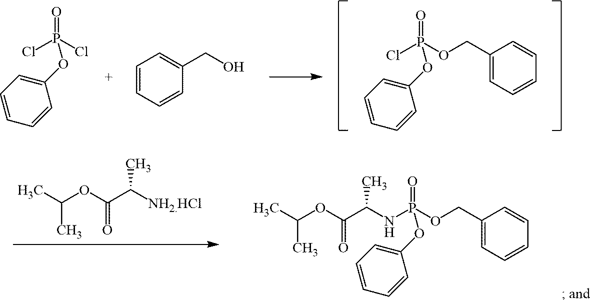

(1.b) debenzylation of isopropyl ((benzyloxy)(phenoxy)phosphoryl)-L-alaninate and in situ reduction of quinine to afford the dihydroquinine salt of isopropyl (hydroxy(phenoxy)phosphoryl)-L-alaninate:

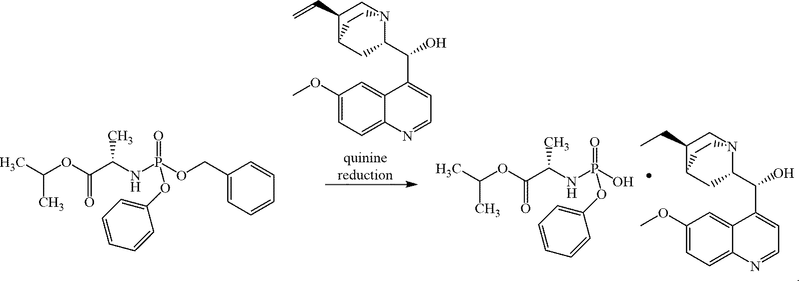

In one embodiment, the debenzylation of isopropyl ((benzyloxy)(phenoxy)phosphoryl)-L-alaninate and in situ reduction of quinine is conducted in the presence of Pd/C and H₂. In an alternative embodiment the debenzylation and reduction are conducted in the presence of a metal catalyst and H₂. In an additional alternative embodiment, debenzylation and reduction are conducted in the presence of a metal catalyst and a reductant. Reductants suitable for the transformation include but are not limited to formate salts, Hantzsch ester and derivatives thereof, and cyclohexadiene and derivatives thereof.

In an alternative embodiment, the debenzylation of isopropyl ((benzyloxy)(phenoxy)phosphoryl)-L-alaninate is conducted in the presence of dihydroquinine to afford the dihydroquinine salt of isopropyl (hydroxy(phenoxy)phosphoryl)-L-alaninate:

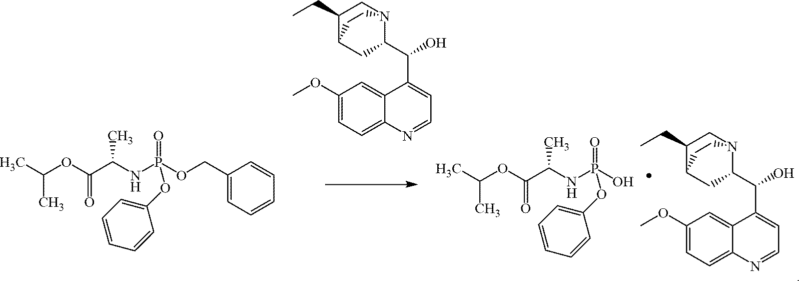

In certain embodiments, the dihydroquinine added to the debenzylation reaction is prepared separately by the reduction of quinine.

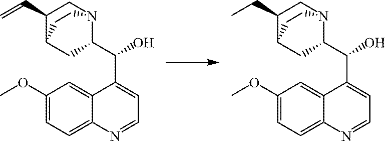 [SMILES: C=C[C@H]1C[N@]2CC[C@H]1C[C@H]2[C@H](O)c1ccnc2ccc(OC)cc12 ; CC[C@H]1C[N@]2CC[C@H]1C[C@H]2[C@H](O)c1ccnc2ccc(OC)cc12]

In certain embodiments, the quinine that is reduced to dihydroquinine is a salt. In certain embodiments, the salt is the hemisulfate. In certain embodiments, the quinine is the hemisulfate monohydrate.

In certain embodiments, separate preparation of the dihydroquinine used in step (1.b) results in improved impurity control.

In some embodiments, step (1.a) is performed in isopropyl acetate solvent. In some embodiments, step (1.a) is performed in an alkyl acetate solvent. In some embodiments step (1.a) is performed in a polar aprotic organic solvent. Alternative solvents suitable for use in step (1.a) include, but are not limited to, dichloromethane, acetonitrile, tetrachloroethane, benzene, chlorobenzene, toluene, trifluorotoluene, isopropyl acetate, ethyl acetate, tetrahydrofuran, diethyl ether, methyl tertbutyl ether, dimethoxy ethane, dimethyl acetamide, and N-methyl-2-pyrrolidone.

Step (1.a) can be performed below room temperature. In certain embodiments, step (1.a) is performed at or below about -70° C. In certain embodiments, step (1.a) is performed at or below about -50° C. In certain embodiments, step (1.a) is performed at or below about -30° C. In certain embodiments, step (1.a) is performed at or below about -10° C. In certain embodiments, step (1.a) is performed at or below about 0° C. In certain embodiments, step (1.a) is performed at or below about 20° C. Alternatively, step (1.a) can be run at any temperature that achieves the desired result.

In some embodiments, the debenzylation step of (1.b) is performed in isopropyl alcohol solvent. In some embodiments, the debenzylation is performed in an alkyl alcohol solvent. In some embodiments, the benzylation is performed in a polar protic solvent. In some embodiments, the benzylation is performed in a polar solvent. Alternative solvents suitable for use in step (1.b) include, but are not limited to, water, methanol, ethanol, n-propyl alcohol, butanol, pentanol, hexanol, dimethylsulfoxide, tetrahydrofuran, dimethylformamide, dimethylacetamide, dimethoxyethane, dimethyl carbonate, acetonitrile, and N-methyl-2-pyrrolidone.

Step (1.b) can be run at any temperature that achieves the desired result. In some embodiments, step (1.b) is performed at or below about -20° C. In some embodiments, step (1.b) is performed at or below about 0° C. In some embodiments, step (1.b) is performed at or below about 10° C. In some embodiments, step (1.b) is performed between about 10° C. and about 30° C. In some embodiments, step (1.b) is performed at or above about 30° C. In some embodiments, step (1.b) is performed at or above about 50° C. In some embodiments, step (1.b) is performed at or above about 70° C.

In a further aspect of the present invention, the synthesis process of Compound 2 comprises the steps below:

Step 1 (1.2.a): the protection of the 5′-hydroxyl and the 3′-hydroxyl of the nucleoside (3S,4R,5R)-3,4-dihydroxy-5-(hydroxymethyl)-3-methyldihydrofuran-2(3H)-one to afford a carbonate or carbamate compound of Formula I wherein R^{1a} and R^{1b} are oxygen protecting groups and at least one of R^{1a} and R^{1b} is —C(O)OC₁-₆alkyl, —C(O)O-benzyl, or —CH₂-phenyl wherein the phenyl group is substituted or in an alternative embodiment, at least one of R^{1a} and R^{1b} are —C(O)OC₁₋₂₀alkyl, —C(O)OC₂₋₂₀alkenyl, or —C(O)NR^{10a}R^{10b} wherein R^{10a} and R^{10b} are independently selected from hydrogen, C₁₋₂₀alkyl, C₂₋₂₀alkenyl, aryl, arylalkyl, heteroaryl, and heteroarylalkyl and wherein the aryl, arylalkyl, heteroaryl, and heteroarylalkyl can optionally be substituted with at least one substituent selected from alkoxy (including but not limited to methoxy and ethoxy), hydroxy, nitro, bromo, chloro, fluoro, azido, and haloalkyl:

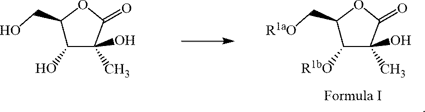

In one embodiment, at least one of R^{1a} and R^{1b} is —CH₂-phenyl wherein the phenyl group is substituted with at least one substitutent selected from alkoxy, hydroxy, nitro, bromo, chloro, fluoro, azido, and haloalkyl.

Non-limiting examples of substituted benzyl ether moieties include p-methoxybenzyl, 3,4-dimethoxybenzyl, 2,4-dimethoxybenzyl, 2-hydroxybenzy, 3,4-dimethoxybenzyl, 2,3,4-trimethoxybenzyl, 3,4,5-trimethoxybenzyl, 2,5-dimethoxybenzyl, o-nitrobenzyl, p-nitrobenzyl, p-bromobenzyl, p-chlorobenzyl, 2,6-dichlorobenzyl, p-phenylbenzyl, 2,6-difluorobenzyl, p-azidobenzyl, 2-trifluorobenzyl, and 4-azido-3-chlorobenzyl.

In one embodiment, at least one of R^{1a} and R^{1b} is —C(O)OC₁₋₆alkyl, for example —C(O)OtBu, or —C(O)O-benzyl. In an alternative embodiment, at least one of R^{1a} and R^{1b} is —C(O)OCH₃. In an alternative embodiment, both R^{1a} and R^{1b} are —C(O)OCH₃.

In one embodiment, R^{1a} is —C(O)OC₁₋₆alkyl, —C(O)O-benzyl, or —CH₂-phenyl wherein the phenyl group is substituted and R^{1b} is an oxygen protecting group which when attached to the oxygen is an ester, ether, or silyl ether moiety. In an alternative embodiment, R^{1b} is —C(O)OC₁₋₆alkyl, —C(O)O-benzyl, or —CH₂-phenyl wherein the phenyl group is substituted and R^{1a} is an oxygen protecting group which when attached to the oxygen is an ester, ether, or silyl ether moiety.

In an alternative embodiment, at least one of R^{1a} and R^{1b} are —C(O)OC₁₋₂₀alkyl, including —C(O)OC₁₋₁₈alkyl, —C(O)OC₁₋₁₆alkyl, —C(O)OC₁₋₁₄alkyl, —C(O)OC₁₋₁₂alkyl, —C(O)OC₁₋₁₀alkyl, —C(O)OC₁₋₈alkyl, —C(O)OC₁₋₆alkyl, —C(O)OC₁₋₄alkyl, —C(O)OC₁₋₂alkyl, —C(O)OC₂₋₂₀alkyl, —C(O)OC₄₋₂₀alkyl, —C(O)OC₆₋₂₀alkyl, —C(O)OC₈₋₂₀alkyl, —C(O)OC₁₀₋₂₀alkyl, —C(O)OC₁₂₋₂₀alkyl, —C(O)OC₁₄₋₂₀alkyl, —C(O)OC₁₆₋₂₀alkyl, and —C(O)OC₁₈₋₂₀alkyl. In one embodiment, both R^{1a} and R^{1b} are —C(O)OC₁₋₂₀alkyl. In one embodiment, both R^{1a} and R^{1b} are —C(O)OC₁₆H₃₃.

In an alternative embodiment, R^{1a} and R^{1b} are both —C(O)NR^{10a}R^{10b}, for example —C(O)NHPh, —C(O)NHBn, —C(O)N(Ph)₂, —C(O)N(Bn)₂, —C(O)NHC₁₋₂₀alkyl (including, but not limited to, —C(O)NHCH₃, —C(O)NHtBu, and —C(O)NHC₁₆H₃₃), and —C(O)N(C₁₋₂₀alkyl)₂ including, but not limited to, —C(O)N(CH₃)₂, —C(O)N(tBu)₂, and —C(O)N(C₁₆H₃₃)₂). In one embodiment, R^{1a} and R^{1b} are both —C(O)NR^{10a}R^{10b}. In one embodiment, R^{1a} is —C(O)NR^{10a}R^{10b} and R^{1b} is an oxygen protecting group which when attached to the oxygen is an ester, ether, or silyl ether moiety. In an alternative embodiment, R^{1b} is —C(O)NR^{10a}R^{10b} and R^{2a} is an oxygen protecting group which when attached to the oxygen is an ester, ether, or silyl ether moiety.

In one embodiment, at least one of R^{1a} and R^{1b} are —C(O)NHC₁₋₂₀alkyl, including —C(O)NHC₁₋₁₈alkyl, —C(O)NHC₁₋₁₆alkyl, —C(O)NHC₁₋₁₄alkyl, —C(O)NHC₁₋₁₂alkyl, —C(O)NHC₁₋₁₀alkyl, —C(O)NHC₁₋₈alkyl, —C(O)NHC₁₋₆alkyl, —C(O)NHC₁₋₄alkyl, —C(O)NHC₁₋₂alkyl, —C(O)NHC₂₋₂₀alkyl, —C(O)NHC₄₋₂₀alkyl, —C(O)NHC₆₋₂₀alkyl, —C(O)NHC₈₋₂₀alkyl, —C(O)NHC₁₀₋₂₀alkyl, —C(O)NHC₁₂₋₂₀alkyl, —C(O)NHC₁₄₋₂₀alkyl, —C(O)NHC₁₆₋₂₀alkyl, and —C(O)NHC₁₈₋₂₀alkyl. In one embodiment, both R^{1a} and R^{1b} are —C(O)NHC₁₋₂₀alkyl. In one embodiment, both R^{1a} and R^{1b} are —C(O)NHC₁₆H₃₃.

This step may be conducted according to one of the procedures described in Theodora W. Green, Protective Groups in Organic Synthesis, Third Edition, John Wiley & Sons (1999), which is incorporated by reference, for the protection of hydroxyls. For example, when R^{1a} and/or R^{1b} is —CH₂-phenyl wherein the phenyl group is substituted, the compound of Formula I can be prepared according to the conditions described in the text on pages 76-99. When R^{1a} and/or R^{1b} is —C(O)OCH₃, the compound of Formula I can be prepared using ClC(O)OCH₃ in a base such as triethylamine and an appropriate solvent, such as THF.

In an alternative embodiment, the 5′ - and 3′-hydroxyl groups on the nucleoside (3S,4R,5R)-3,4-dihydroxy-5-(hydroxymethyl)-3-methyldihydrofuran-2(3H)-one are protected with protecting groups R^{1c} and R^{1d} wherein R^{1c} and R^{1d} are independently selected from an oxygen protecting group which when attached to the oxygen is an ester, ether, or silyl ether moiety to afford a compound of Formula I′:

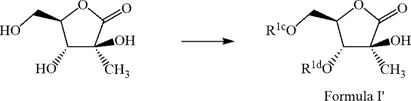

For example, the protecting group that when attached to the oxygen can be an ester moiety, for example benzoate acetate. In one embodiment, the oxygen protecting group that when attached to the oxygen is a silyl ether moiety (for example (trimethylsilyl (TMS), triisopropylsilyl (TIPS), tert-butyldimethylsilyl (TBDMS or TBS) or tert-butyldiphenylsilyl (TBDPS). In one embodiment, the oxygen protecting group that when attached to the oxygen is an ether moiety, for example methyl ether, methoxymethyl ether, or benzyl ether. These protecting groups can be installed according to one of the procedures described in Theodora W. Green, Protective Groups in Organic Synthesis, Third Edition, John Wiley & Sons (1999), which is incorporated by reference, for the protection of hydroxyls. For example, when the oxygen protecting group which when attached to the oxygen is an ester moiety, the compound of Formula I can be prepared according to the conditions described in the text on page 149-178 and when the oxygen protecting group is a silyl ether moiety when attached to the oxygen, the compound of Formula I can be prepared according to the conditions described in the text on page 113-147. In one embodiment, the protecting group is a tert-butyldimethylsilyl (TBS) group. The TBS group is selectively installed on the primary alcohol over the secondary alcohol using the conditions described in the text on page 128 and in Ogilvie et al. Can. J. Chem. 1979, 57, 2230. These conditions include the use of TBSCl, DMAP, and NEt₃ in DMF at 25° C.

Non-limiting examples of additional protecting groups which when attached to the oxygen also include -bromobenzoate, p-methoxybenzyloxymethyl ether (MPBM), o-nitrobenzyloxymethyl ether (NBOM), p-nitrobenzyloxymethyl ether, t-butoxymethyl ether, 2,2,2-trichloroethoxymethyl ether, 3-bromotetrahydropyranyl ether, tetrahydropyranyl ether, tetrahydrothiopyranyl ether, 1-methoxycyclohexyl ether, 1,4-dioxan-2-yl ether, tetrahydrofuranyl ether, tetrahydrothiofuranyl ether, a substituted phenyl ether, 2-picolyl ether, 4-picolyl ether, 1,3-benzodithiolan-2-yl ether, p-chlorophenoxyacetate ester, 3-phenylpropionate ester, p-phenylbenzoate ester, alkyl p-nitrophenyl carbonyl, alkyl benzyl carbonyl, alkylp-methoxybenzyl carbonyl, alkyl o-nitrobenzyl carbonyl, and alkyl p-nitrobenzyl carbonyl.

Non-limiting examples of R^{1a} and R^{1b} include:

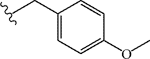

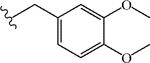

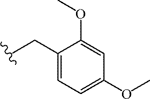

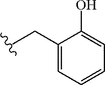

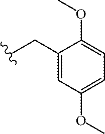

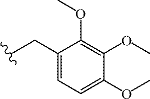

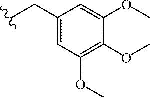

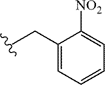

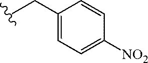

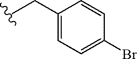

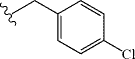

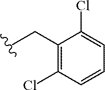

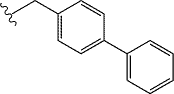

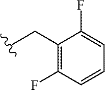

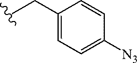

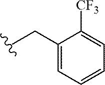

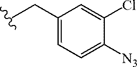

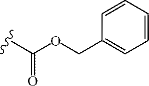

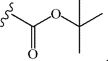

Additional non-limiting examples of R^{1a} and R^{1b} include:

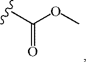

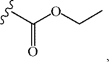

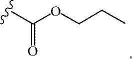

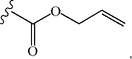

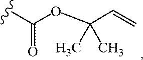

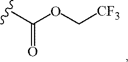

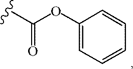

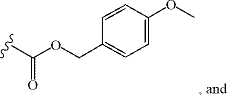

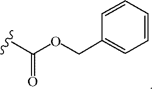

Additional non-limiting examples of R^{1a} and R^{1b} include:

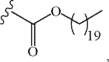

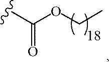

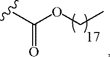

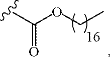

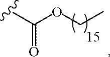

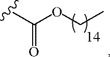

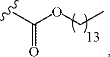

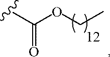

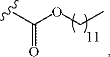

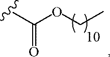

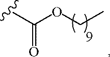

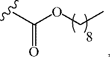

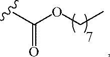

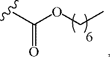

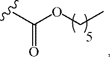

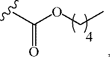

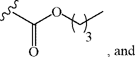

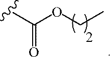

Additional non-limiting examples of R^{1a} and R^{1b} include:

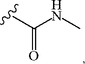

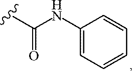

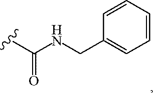

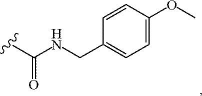

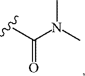

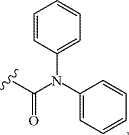

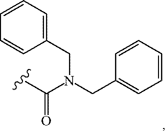

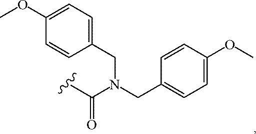

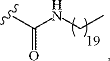

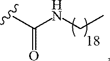

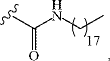

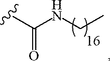

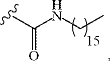

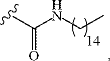

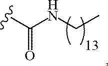

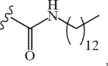

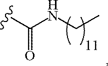

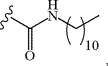

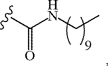

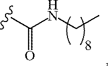

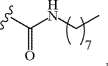

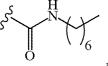

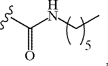

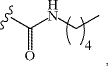

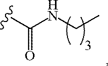

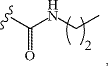

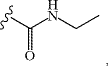

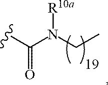

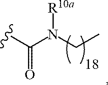

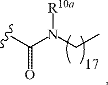

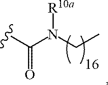

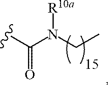

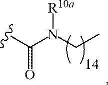

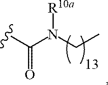

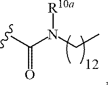

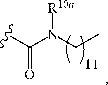

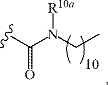

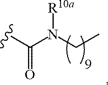

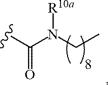

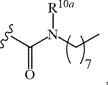

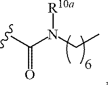

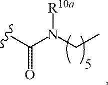

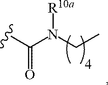

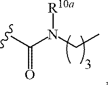

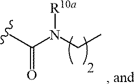

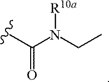

In one embodiment, R^{10a} is hydrogen. In one embodiment, R^{10a} is phenyl.

Non-limiting examples of a carbonate compound of Formula I include:

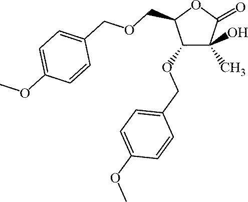 [SMILES: COc1ccc(COC[C@H]2OC(=O)[C@@](C)(O)[C@@H]2OCc2ccc(OC)cc2)cc1]

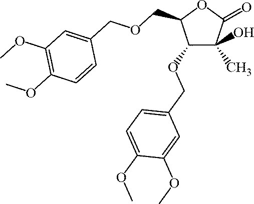 [SMILES: COc1ccc(COC[C@H]2OC(=O)[C@@](C)(O)[C@@H]2OCc2ccc(OC)c(OC)c2)cc1OC]

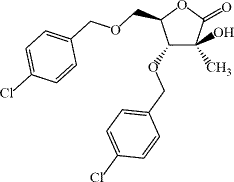 [SMILES: C[C@@]1(O)C(=O)O[C@H](COCc2ccc(Cl)cc2)[C@H]1OCc1ccc(Cl)cc1]

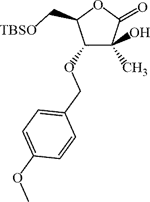 [SMILES: COc1ccc(CO[C@@H]2[C@@H](CO[Si](C)(C)C(C)(C)C)OC(=O)[C@@]2(C)O)cc1]

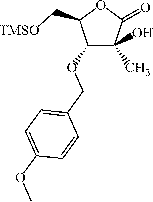

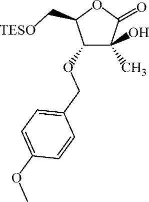

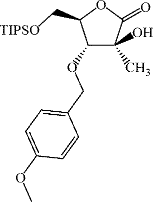

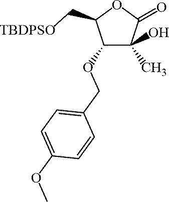

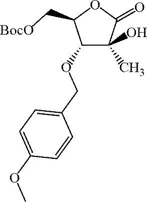

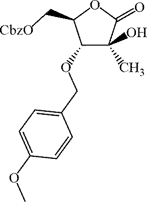

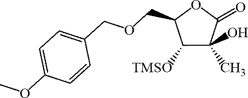

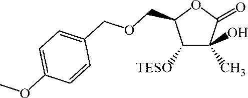

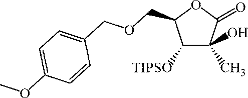

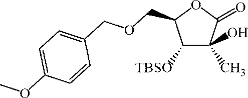 [SMILES: COc1ccc(COC[C@H]2OC(=O)[C@@](C)(O)[C@@H]2O[Si](C)(C)C(C)(C)C)cc1]

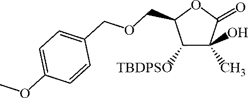

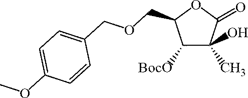

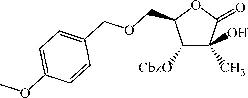

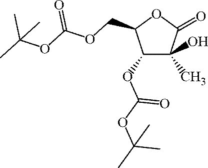 [SMILES: CC(C)(C)OC(=O)OC[C@H]1OC(=O)[C@@](C)(O)[C@@H]1OC(=O)OC(C)(C)C]

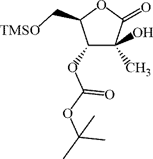

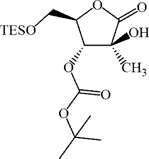

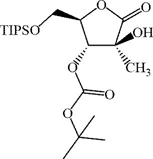

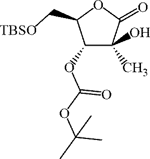 [SMILES: CC(C)(C)OC(=O)O[C@@H]1[C@@H](CO[Si](C)(C)C(C)(C)C)OC(=O)[C@@]1(C)O]

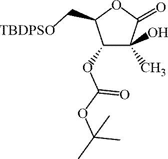

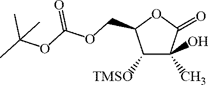

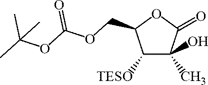

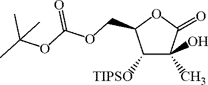

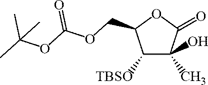 [SMILES: CC(C)(C)OC(=O)OC[C@H]1OC(=O)[C@@](C)(O)[C@@H]1O[Si](C)(C)C(C)(C)C]

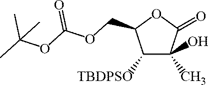

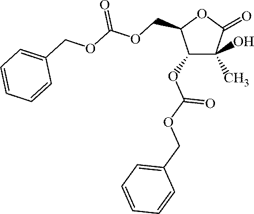 [SMILES: C[C@@]1(O)C(=O)O[C@H](COC(=O)OCc2ccccc2)[C@H]1OC(=O)OCc1ccccc1]

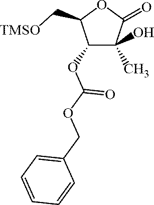

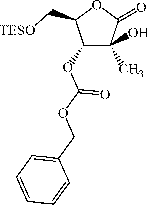

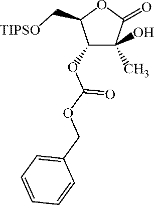

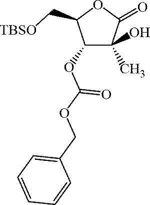 [SMILES: CC(C)(C)[Si](C)(C)OC[C@H]1OC(=O)[C@@](C)(O)[C@@H]1OC(=O)OCc1ccccc1]

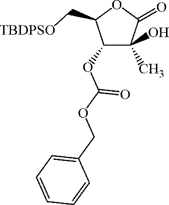

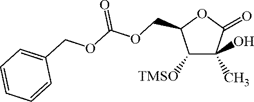

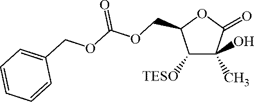

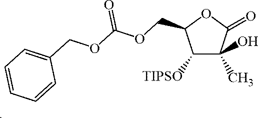

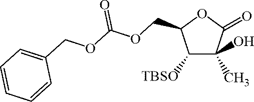 [SMILES: CC(C)(C)[Si](C)(C)O[C@@H]1[C@@H](COC(=O)OCc2ccccc2)OC(=O)[C@@]1(C)O]

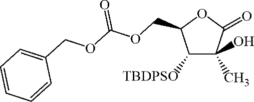

Additional non-limiting examples of a carbonate compound of Formula I include:

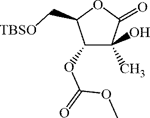 [SMILES: COC(=O)O[C@@H]1[C@@H](CO[Si](C)(C)C(C)(C)C)OC(=O)[C@@]1(C)O]

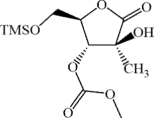

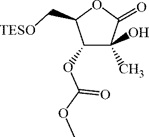

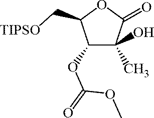

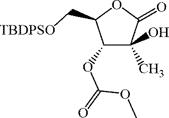

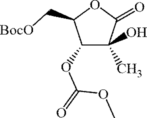

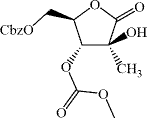

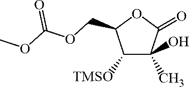

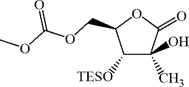

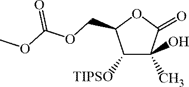

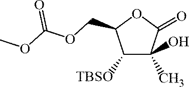 [SMILES: COC(=O)OC[C@H]1OC(=O)[C@@](C)(O)[C@@H]1O[Si](C)(C)C(C)(C)C]

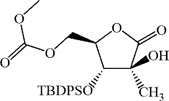

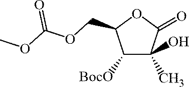

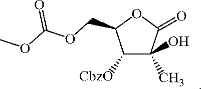

Additional non-limiting examples of a carbonate compound of Formula I include:

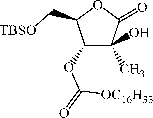

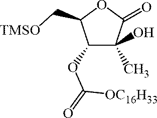

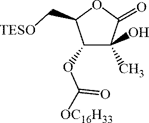

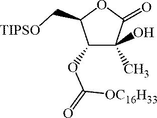

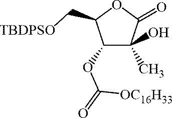

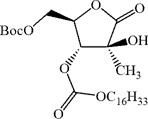

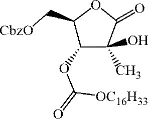

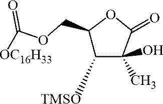

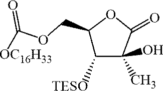

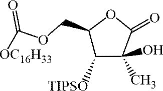

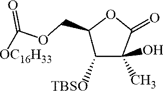

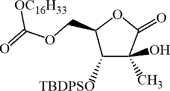

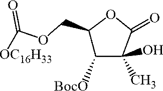

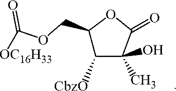

Additional non-limiting examples of a compound of Formula I include:

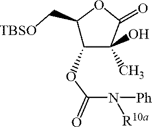

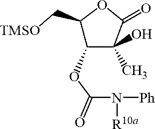

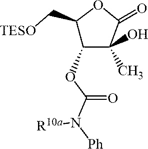

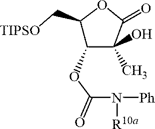

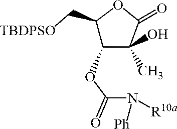

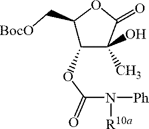

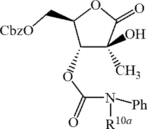

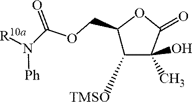

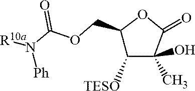

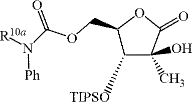

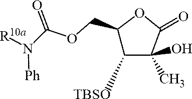

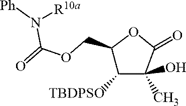

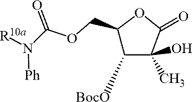

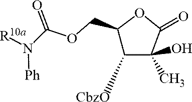

In one embodiment, R^{10a} is hydrogen. In one embodiment, R^{10a} is phenyl.

Step (1.2.b): the conversion of the alcohol of Formula I into the corresponding fluoro derivative with inversion of stereochemistry to afford a carbonate or carbamate compound of Formula II with an appropriate fluorinating agent such as Morpho-DAST, DAST or SO₂F₂:

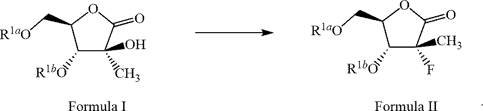

In one embodiment, step (1.2.b) as described herein is conducted with a sulfonyl fluoride/TREAT•HF mixture (SO₂F₂, NEt₃·3HF). In one embodiment, step (1.2.b) as described herein is conducted with DAST (Et₂NSF₃). In one embodiment, step (1.2.b) as described herein is conducted with Deoxo-Fluor®. In one embodiment, step (1.2.b) as described herein is conducted with morpholinosulfur trifluoride (Morph-DAST). Alternatively, step (1.2.b) can be performed with any fluorinating reagent that achieves the desired result.

In some embodiments step (1.2.b) is performed at or below about -70° C. In some embodiments step (1.2.b) is performed at or below about -50° C. In some embodiments step (1.2.b) is performed at or below about -10° C. In some embodiments step (1.2.b) is performed at or below about 0° C. In some embodiments step (1.2.b) is performed at or below about 10° C. In some embodiments step (1.2.b) is performed between about 10° C. and about 30° C. In some embodiments step (1.2.b) is performed at or above about 30° C. In some embodiments step (1.2.b) is performed at or above about 50° C. Step (1.2.b) can be performed at any temperature that achieves the desired result.

In an alternative embodiment, the fluorination reaction primarily proceeds with retention of stereochemistry at the 2′-position. In this embodiment, a compound of Formula I″ is reacted with a fluorination reagent to afford a compound of Formula II:

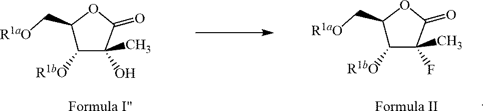

If the product of the fluorination reaction is a mixture of “α-fluoro” and “β-fluoro” lactone derivatives, the compounds can be separated by conventional methods known to a skilled artisan, for example, column chromatography or crystallization, to isolate the desired stereochemistry (“α-fluoro” configuration).

Additional non-limiting examples of nucleophilic fluorination reagents include pyridinium poly(hydrogen fluoride) (Olah’s reagent), nitrosonium tetrafluoroborate/pyridinium poly(hydrogen fluoride), triethylamine tris(hydrogen fluorine) (TREAT•HF), perfluoro-1-butanesulfonyl fluoride (PBSF), Yarovenko’s reagent, Ishikawa’s reagent, TFEDMA, N,N′-dimethyl-2,2,-difluroimidazolidine, 4-morpholinosulfur trifluoride, bromine trifluoride, and 4-tert-butyl-2,6-dimethylphenylsulfur trifluoride (Fluolead™). The fluorination reaction can be conducted according to conditions described in Pankiewicz, K., Journal of Fluorine Chemistry, 1993, 64, 15-36; Hudlicky, M. “Fluorination with Diethylaminosulfur Trifluoride and related Aminofluorosulfuranes” in Organic Reactions, Vol. 35, 1998, 513-637; Singh et al. Synthesis, 2002, 17, 2561-2578; and, Liang, Theresa, et al. Angewandte Chemie International Edition, 2013, 52, 8214-8264.

Step (1.2.c): the reduction of the lactone of the nucleoside compound of Formula II to afford the nucleoside compound of Formula III, with an appropriate reducing agent, for example Red-Al, DIBAL, LiAlH₄ or NaBH₄;

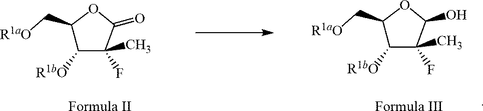

Non-limiting reagents for the reduction of the lactone as described herein include DIBAL-H (diisobutylaluminium hydride), NaBH₄, Red-Al® sodium bis(2-methoxyethoxy)aluminum hydride, and LiAlH₄ (lithium aluminum hydride). Alternatively, the reduction of the lactone can be achieved by metal reductants, including but not limited to zinc, magnesium, copper, iron, sodium, potassium, and lithium. Any reductant can be used which achieves the desired results.

In some embodiments step (1.2.c) is performed at or below about -70° C. In some embodiments step (1.2.c) is performed at or below about -50° C. In some embodiments step (1.2.c) is performed at or below about -10° C. In some embodiments step (1.2.c) is performed at or below about 0° C. In some embodiments step (1.2.c) is performed at or below about 10° C. In some embodiments step (1.2.c) is performed between about 10° C. and about 30° C. In some embodiments step (1.2.c) is performed at or above about 30° C. In some embodiments step (1.2.c) is performed at or above about 50° C. Step (1.2.c) can be performed at any temperature that achieves the desired result.

If the product of the reduction is a mixture of nucleosides with R- and S-stereochemistry at the hydroxyl group, the compounds can be separated by conventional methods known to a skilled artisan, for example, column chromatography or crystallization, to isolate the desired stereochemistry. Alternatively, the mixture of diastereomers can be carried forward in Step (1.2.d) as described herein, resulting in a compound of Formula IV as a mixture of diastereomers. In this embodiment, the compound of Formula IV (as a mixture of diastereomers at the 1′-position) is reacted with 2-amino-6-chloropurine to afford a compound of Formula V as a mixture of diastereomers. The Formula V diastereomers can be separated by conventional methods known to a skilled artisan, for example, column chromatography or crystallization, to isolate the desired stereochemistry.

Step (1.2.d): the conversion of a compound of Formula III to a compound of Formula IV wherein X is Cl, Br, or OAc:

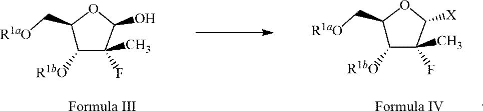

In one embodiment, the hydroxyl group is converted to a Br using PPh₃ and CBr₄. In one embodiment, the hydroxyl group is converted to a Br using PPh₃ and dibromohydantoin. In one embodiment, the hydroxyl group is converted to Cl using PPh₃ and CCl₄. In one embodiment, the hydroxyl group is converted to OAc using ClC(O)CH₃ and, optionally NEt₃. Step (1.2.d) can be accomplished by any chlorinating, brominating, or acetylating reagent that achieves the desired result.

In some embodiments, step (1.2.d) is performed in tetrahydrofuran solvent. In some embodiments, step (1.2.d) is performed in an ether solvent. In some embodiments, step (1.2.d) is performed in a non-polar solvent. In some embodiments, step (1.2.d) is performed in a polar protic solvent. Solvents suitable for use in step (1.2.d) include, but are not limited to, diethyl ether, methyl tertbutyl ether, tetrahydrofuran, dimethoxy ethane, methanol, ethanol, trifluoroethanol, propanol, butanol, pentanol, hexanol, pentane, hexane, heptane, benzene, toluene, trifluorotoluene, and xylene.

In some embodiments step (1.2.d) is performed at or below about -70° C. In some embodiments step (1.2.d) is performed at or below about -50° C. In some embodiments step (1.2.d) is performed at or below about -10° C. In some embodiments step (1.2.d) is performed at or below about 0° C. In some embodiments step (1.2.d) is performed at or below about 10° C. In some embodiments step (1.2.d) is performed between about 10° C. and about 30° C. In some embodiments step (1.2.d) is performed at or above about 30° C. In some embodiments step (1.2.d) is performed at or above about 50° C. Step (1.2.d) can be performed at any temperature that achieves the desired result.

Step (1.2.e): the nucleophilic substitution of the compound of Formula IV with 2-amino-6-chloropurine to afford a compound of Formula V:

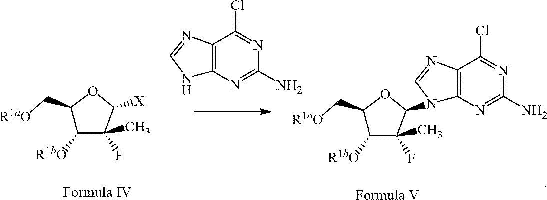

In one embodiment, the nucleophilic substitution in Step (1.2.e) as described herein is conducted with a non-nucleophilic base. Non-limiting embodiments of non-nucleophilic bases for Step (1.2.e) include sodium tert-pentoxide, potassium tert-pentoxide, sodium tert-butoxide, potassium tert-butoxide, lithium diisopropylamide, and lithium bis(trimethylsilyl)amide. In one embodiment, the base in Step (1.2.e) as described herein is sodium tert-butoxide or potassium tert-butoxide. In one embodiment, the base in Step (1.2.e) as described herein is sodium tert-pentoxide or potassium tert-pentoxide. Any base can be used in step (1.2.e) that achieves the desired result.

In some embodiments, the reaction of step (1.2.e) is performed in acetonitrile solvent. In some embodiments the solvent of step (1.2.e) is selected from acetonitrile, tetrahydrofuran, dimethylformamide, dimethylacetamide, dimethylsulfoxide, and dimethoxyethane. In some embodiments the reaction of step (1.2.e) is performed in a polar aprotic solvent.

In some embodiments, the compound of Formula V can be purified from a crude reaction mixture by selective crystallization. In some embodiments, the selective crystallization is performed with a mixture of solvents. In some embodiments, the mixture of solvents used is a mixture of DCM and n-heptane.

In some embodiments step (1.2.e) is performed between about 10° C. and about 30° C. In some embodiments step (1.2.e) is performed at or above about 30° C. In some embodiments step (1.2.e) is performed at or above about 50° C. In some embodiments step (1.2.e) is performed at or above about 70° C. In some embodiments step (1.2.e) is performed at or above about 90° C. Step (1.2.e) can be performed at any temperature that achieves the desired result.

In certain embodiments, the invention includes the crystalline compound of Formula V of structure:

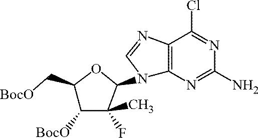

Step (1.2.f): the conversion of the 2-amino-6-chloropurine base to the 2-amino-N⁶-methyl base and the deprotection of the 3′ and 5′-positions to afford Compound 2:

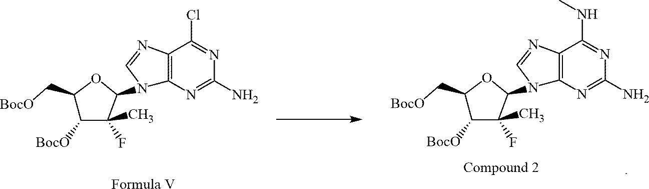

In some embodiments, step (1.2.f) is performed with methylamine. In some embodiments the methylamine used is a solution in methanol. In some embodiments the methylamine used is a solution in water.

In some embodiments, the reaction of step (1.2.f) is performed in acetonitrile solvent. In some embodiments the solvent of step (1.2.f) is selected from acetonitrile, tetrahydrofuran, dimethylformamide, dimethylacetamide, dimethylsulfoxide, dimethoxyethane, methanol, ethanol, propanol, butanol, pentanol, and hexanol. In some embodiments the reaction of step (1.2.f) is performed in a polar aprotic solvent. In some embodiments the reaction of step (1.2.f) is performed in a polar protic solvent.

In some embodiments step (1.2.f) is performed between about 10° C. and about 30° C. In some embodiments step (1.2.f) is performed at or above about 30° C. In some embodiments step (1.2.f) is performed at or above about 50° C. In some embodiments step (1.2.f) is performed at or above about 70° C. In some embodiments step (1.2.f) is performed at or above about 90° C. Step (1.2.f) can be performed at any temperature that achieves the desired result.

In some embodiments, step (1.2.f) comprises at least two smaller steps, with optional purification of the products between the steps. In certain embodiments, step (1.2.f) can be broken into two steps. In some embodiments of step (1.2.f), the compound of Formula V is first converted to a compound of Formula Va, leaving the alcohol protecting groups intact.

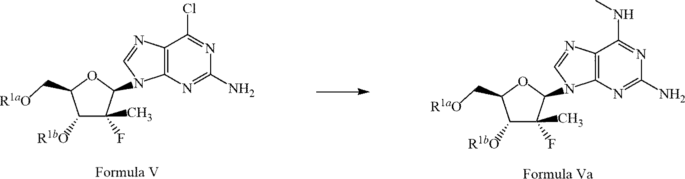

Compound of Formula Va are optionally purified by crystallization. In some embodiments, the crude compound of Formula Va is carried forward without additional purification. In certain embodiments, it is advantageous to purify the intermediate compound of Formula Va. In certain embodiments, the invention includes the crystalline compound of Formula Va of structure:

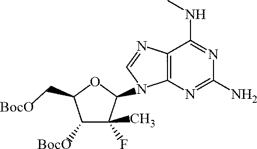

In some embodiments of step (1.2.f), the compound of Formula Va is deprotected to give Compound 2. In certain embodiments, the deprotection is carried out under acidic conditions. In certain embodiments, the deprotection is carried out under basic conditions. When basic conditions are used for the deprotection, there is no need for a step to neutralize the salt before the next reaction.

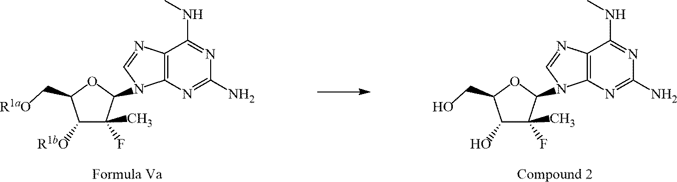

In an alternative embodiment, the 2-amino-6-chloropurine base is converted to the 2-amino-N⁶-methyl base and the 5′-hydroxyl group is selectively deprotected, as shown in step (1.2.f.1):

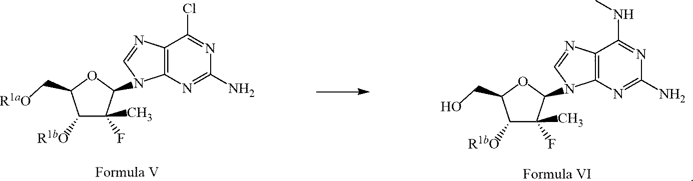

In certain embodiments, a mixture of products obtained from step (1.2.f) can be taken to the next step without further purification. In certain embodiments, a mixture of products is taken to the next step without further purification when the product of step (1.2.e) was purified.

In this embodiment, a compound of Formula VI is reacted with the dihydroquinine salt of isopropyl (hydroxy(phenoxy)phosphoryl)-L-alaninate in the presence of a specified activator as described herein and base to afford a protected diastereomerically enriched Sₚ-phosphoramidate nucleotide of Formula VII wherein the Sₚ-diastereomer is in excess of the Rₚ-diastereomer:

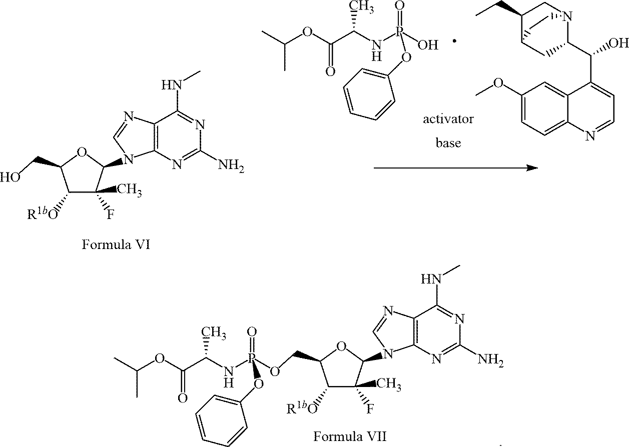

In an alternative embodiment, the N²-position of the nucleoside is protected prior to the phosphorylation. In this embodiment, a compound of Formula VIII where the N²-amine is protected is reacted with the dihydroquinine salt of isopropyl (hydroxy(phenoxy)phosphoryl)-L-alaninate in the presence of a specified activator as described herein and base to afford a protected diastereomerically enriched Sₚ-phosphoramidate nucleotide of Formula IX wherein the Sₚ-diastereomer is in excess of the Rₚ-diastereomer:

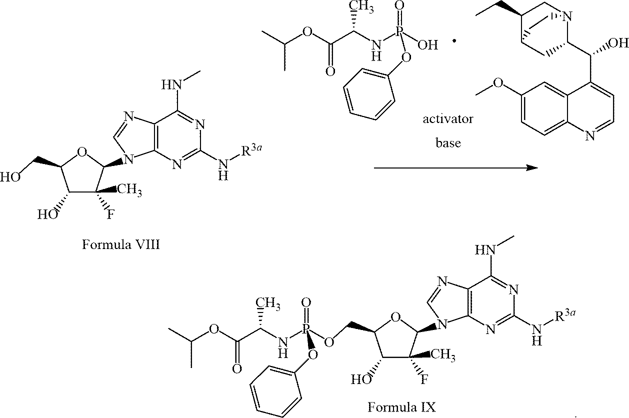

In one embodiment, the manufacture of a compound of Formula VIII comprises the steps: (1.2.f.1) protecting the N²-position in the compound of Formula V with protecting group R^{3a} to afford a compound of Formula X wherein R^{3a} is a nitrogen protecting which when attached to the nitrogen is an amine, amide, or carbamate moiety:

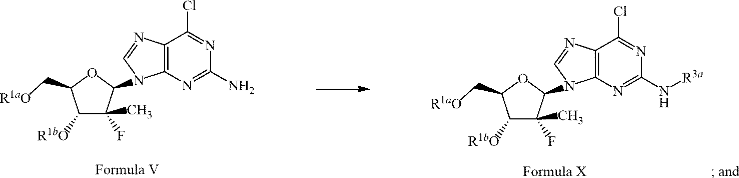

(1.2.g) converting the 6-chloro position in the compound of Formula X to the N⁶-methylamino group and deprotecting the R^{1a} and R^{1b} positions to afford a compound of Formula VIII:

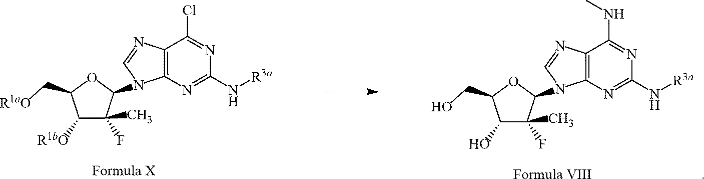

In an additional alternative embodiment, the N²-amine and the N⁶-methylamine of the nucleoside are protected prior to the phosphorylation. In this embodiment, a compound of Formula XI where the N²-amine and the N⁶-methylamine are protected is reacted with the dihydroquinine salt of isopropyl (hydroxy(phenoxy)phosphoryl)-L-alaninate in the presence of a specified activator as described herein and base to afford a protected diastereomerically enriched Sₚ-phosphoramidate nucleotide of Formula XII wherein the Sₚ-diastereomer is in excess of the Rₚ-diastereomer:

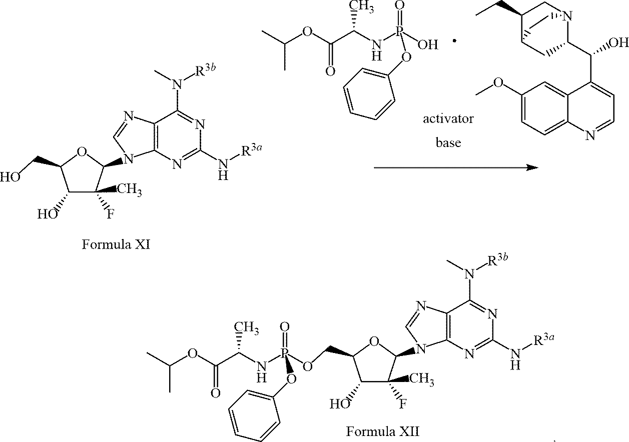

In one embodiment, the manufacture of a compound of Formula XI comprises protecting the N⁶-methylamine in the compound of Formula VIII with protecting group R^{3b} to afford a compound of Formula XI wherein R^{3b} is a nitrogen protecting which when attached to the nitrogen is an amine, amide, or carbamate moiety:

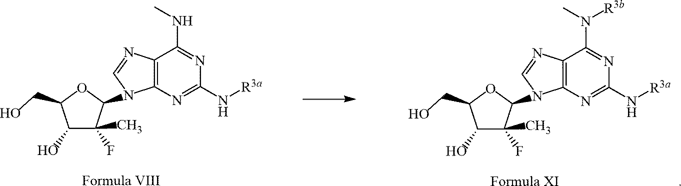

In an alternative embodiment, the N⁶-methylamine of the nucleoside is protected prior to the phosphorylation. In this embodiment, a compound of Formula XIII where the N⁶-methylamine is protected is reacted with the dihydroquinine salt of isopropyl (hydroxy(phenoxy)phosphoryl)-L-alaninate in the presence of a specified activator as described herein and base to afford a protected diastereomerically enriched Sₚ-phosphoramidate nucleotide of Formula XIV wherein the Sₚ-diastereomer is in excess of the Rₚ-diastereomer:

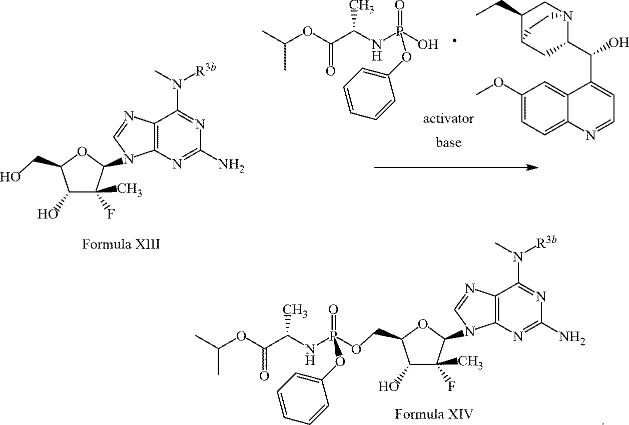

In one embodiment, the manufacture of a compound of Formula XIII is synthesized by protecting the N⁶-methylamine in Compound 2 with protecting group R^{3b} to afford a compound of Formula XIII where the N⁶-methylamine positions:

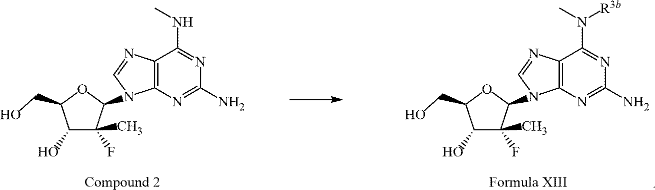

In one embodiment, R^{3a} and R^{3b} are independently nitrogen protecting groups which when attached to the nitrogen are carbamate moieties, for example, tert-butoxycarbonyl-(Boc), benzyloxycarbonyl-(Cbz). In one embodiment, R^{3a} and R^{3b} are independently nitrogen protecting groups which when attached to the nitrogen are amine moieties, for example, benzyl amine or para-methoxybenzyl amine. In some embodiments, R^{3a} and R^{3b} are similar protecting groups to R¹ and can be deprotected by a similar process as discussed herein. In one embodiment, R^{3a} and R^{3b} are a benzyl amine when attached to the nitrogen. The benzyl group can be formed and cleaved as described in Theodora W. Green, Protective Groups in Organic Synthesis, Third Edition, John Wiley & Sons (1999) on pages 579-580. For example, the benzyl group can be installed using BnBr and NEt₃ in CH₃CN and the benzyl group can be removed with Pd/C and HCOOH in CH₃OH. In one embodiment, R^{3a} and R^{3b} are independently a tert-butoxycarbonyl-(Boc) group that is formed and cleaved as described in Theodora W. Green, Protective Groups in Organic Synthesis, Third Edition, John Wiley & Sons (1999) on pages 518-525. For example, the tert-butoxycarbonyl group can be installed using di-tert-butyl-dicarbonate and DMAP in MeCN and can be removed with catalytic DBU in MeOH.

The protected diastereomerically enriched Sₚ-phosphoramidate nucleotides of Formula VII, Formula IX, Formula XII, or Formula XIV are then further optionally purified, e.g., by selective crystallization, to afford the diastereomerically pure Sₚ-purine phosphoramidate nucleotides of Formula VII, Formula IX, Formula XII, or Formula XIV, respectively, wherein the diastereomerically purity is greater than about 90%, about 95% or even about 99% or greater; and then deprotected to afford the diastereomerically pure Sₚ-phosphoramidate nucleotide Compound 1.

In one embodiment, Compound 1 is then further purified and/or converted to a pharmaceutically acceptable salt, for example Compound 1-A.

In an alternative embodiment, the manufacture of Compound 2 comprises the steps (1.2.a) - (1.2.d):
(1.2.a) the protection of nucleoside (3S,4R,5R)-3,4-dihydroxy-5-(hydroxymethyl)-3-methyldihydrofuran-2(3H)-one at the 3′- and 5′-hydroxyl groups wherein the protecting group links together the 3′- and 5′-hydroxyl groups to form a bridge structure:

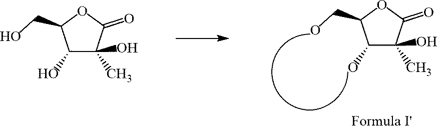

and the bridge structure is selected from

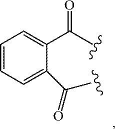

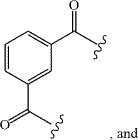

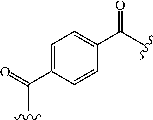

wherein the phenyl group can be substituted with substituents selected from alkoxy (including but not limited to methoxy and ethoxy), hydroxy, nitro, bromo, chloro, fluoro, azido, and haloalkyl.

In an alternative embodiment, the bridge structure is selected from

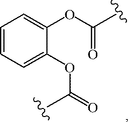

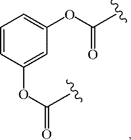

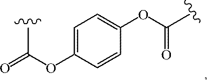

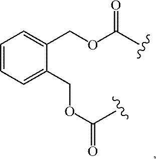

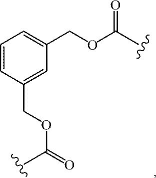

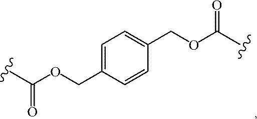

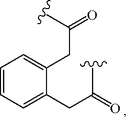

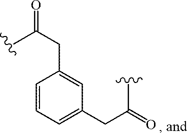

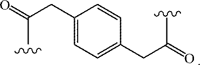

wherein the phenyl group can be substituted with substituents selected from alkoxy (including but not limited to methoxy and ethoxy), hydroxy, nitro, bromo, chloro, fluoro, azido, and haloalkyl.

(1.2.b) the conversion of the alcohol of Formula I′ into a monofluoride with inversion of stereochemistry to afford a compound of Formula II′:

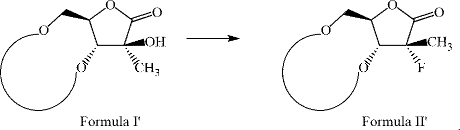

In one embodiment, step (1.2.b) as described herein can be conducted with a sulfonyl fluoride/TREAT•HF mixture (SO₂F₂, NEt₃·3HF). In one embodiment, step (1.2.b) as described herein can be conducted with DAST (Et₂NSF₃). In one embodiment, step (1.2.b) as described herein can be conducted with Deoxo-Fluor®. In one embodiment, step (1.2.b) as described herein can be conducted with morpholinosulfur trifluoride (Morph-DAST).

In an alternative embodiment, the fluorination reaction primarily can proceed with retention of stereochemistry at the 2′-position. In this embodiment, a compound of Formula XV can be reacted with a fluorination reagent to afford a compound of Formula II′:

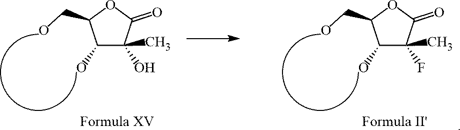

If the product of the fluorination reaction is a mixture of “α-fluoro” and “β-fluoro” lactone derivatives, the compounds can be separated by conventional methods known to a skilled artisan, for example, column chromatography or crystallization, to isolate the desired stereochemistry (“α-fluoro” configuration).

Step (1.2.c): the reduction of the lactone of the nucleoside compound of Formula II′ to afford the nucleoside compound of Formula III′, with an appropriate reducing agent, for example Red-Al, DIBAL, LiAlH₄ or NaBH₄;

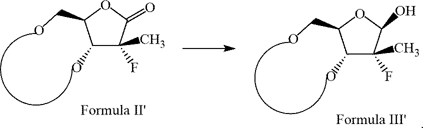

Non-limiting reagents for the reduction of the lactone include DIBAL-H (diisobutylaluminium hydride), NaBH₄, Red-Al® sodium bis(2-methoxyethoxy)aluminum hydride, and LiAlH₄ (lithium aluminum hydride). If the product of the reduction is a mixture of nucleosides with R- and S-stereochemistry at the hydroxyl group, the compounds can be separated by conventional methods known to a skilled artisan, for example, column chromatography or crystallization, to isolate the desired stereochemistry. Alternatively, the mixture of diastereomers can be carried forward in Step (1.2.d), resulting in a compound of Formula IV′ as a mixture of diastereomers. In this embodiment, the compound of Formula IV′ (as a mixture of diastereomers at the 1′-position) can be reacted with 2-amino-6-chloropurine to afford a compound of Formula V as a mixture of diastereomers. The Formula V′ diastereomers can be separated by conventional methods known to a skilled artisan, for example, column chromatography or crystallization, to isolate the desired stereochemistry.

(1.2.d) the conversion of a compound of Formula III′ to a compound of Formula IV′ wherein X is Cl, Br, or OAc:

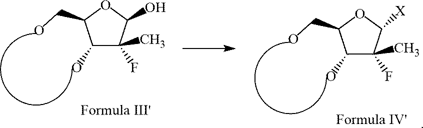

In one embodiment, the hydroxyl group can be converted to a Br using PPh₃ and CBr₄. In one embodiment, the hydroxyl group can be converted to Cl using PPh₃ and CCl₄. In one embodiment, the hydroxyl group can be converted to OAc using ClC(O)CH₃ and, optionally NEt₃.

(1.2.e) the nucleophilic substitution of the compound of Formula IV′ with 2-amino-6-chloropurine to afford a compound of Formula V′:

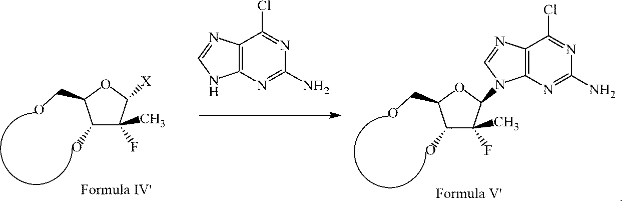

(1.2.f) the conversion of the 2-amino-6-chloropurine base to the 2-amino-N⁶-methyl base and the deprotection of the 3′ and 5′-positions to afford Compound 2:

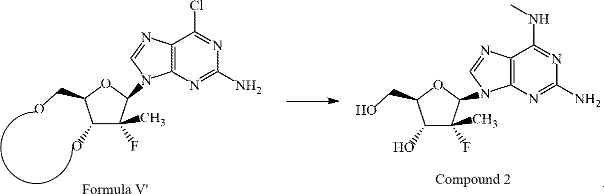

In another aspect of the present invention, a process is provided for the manufacture of a Sₚ-phosphoramidate nucleoside other than the specific phosphoramidate described in the compound illustration. In one embodiment, a process is provided for the manufacture of a phosphoramidate of Formula XVI wherein the Sₚ-isomer is in excess of the Rₚ-isomer:

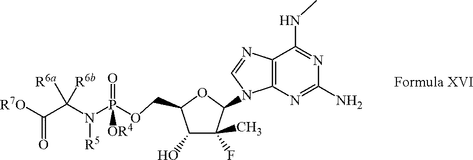

or a pharmaceutically acceptable salt thereof wherein:
R⁴ is hydrogen, C₁₋₆alkyl (including methyl, ethyl, propyl, and isopropyl), C₃₋₇cycloalkyl, or aryl (including phenyl and napthyl);
R⁵ is hydrogen or C₁₋₆alkyl (including methyl, ethyl, propyl, and isopropyl);
R^{6a} and R^{6b} are independently selected from hydrogen, C₁₋₆alkyl (including methyl, ethyl, propyl, and isopropyl), or C₃₋₇cycloalkyl; and
R⁷ is hydrogen, C₁₋₆alkyl (including methyl, ethyl, propyl, and isopropyl), C₁₋₆haloalkyl, or C₃₋₇cycloalkyl.

In one embodiment, the process for the manufacture of the diastereomerically enriched Sₚ-phosphoramidate nucleotide of Formula XVI comprises:
(a) contacting Compound 2 with the dihydroquinine salt of Formula XVII in the presence of a specified activator as described herein and base to afford a diastereomerically enriched Sₚ-phosphoramidate nucleotide of Formula XVI:

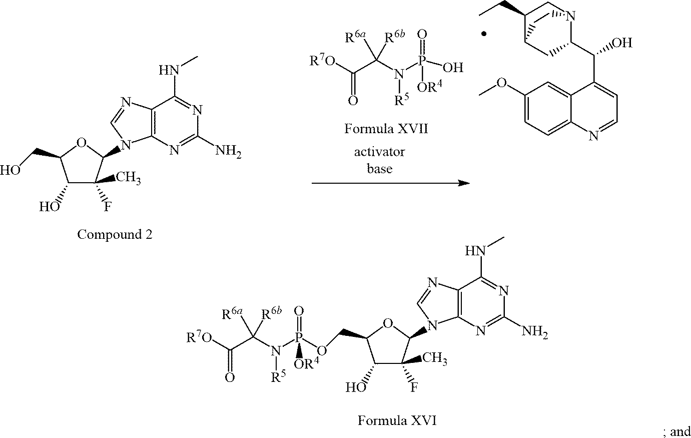

(b) further purifying the diastereomerically enriched Sₚ-phosphoramidate nucleotide of Formula XVI to afford the diastereomerically pure Sₚ-purine phosphoramidate nucleotide of Formula XVI wherein the diastereomerically purity is greater than about 90%, about 95%, or even about 99% or greater; and,
(c) optionally converting the compound of Formula XVI to a pharmaceutically acceptable salt of a compound of Formula XVI;
   wherein R², R⁴, R⁵, R^{6a}, R^{6b}, and R⁷ are as defined herein.

In one embodiment, compounds with an alternative amino acid configuration are synthesized via the processes discussed above:

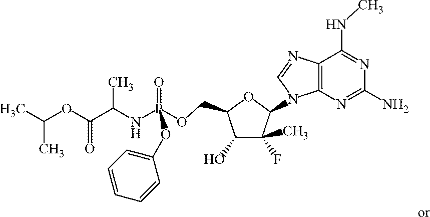

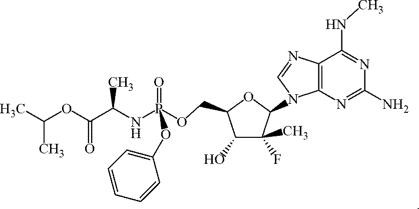

Similarly, the present invention also provides processes for the pharmaceutically acceptable salts of compounds with alterative amino acid configurations, including the hemi-sulfate salt compounds:

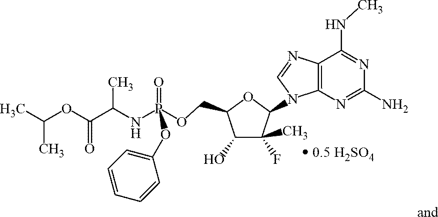

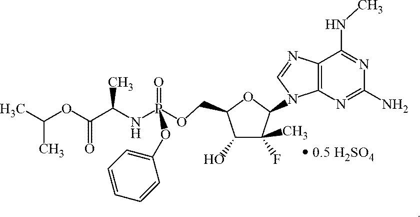

### Definitions

Compounds are described using standard nomenclature. Unless defined otherwise, all technical and scientific terms used herein have the same meaning as is commonly understood by one of skill in the art to which the invention belongs.

The terms “a” and “an” do not denote a limitation of quantity, but rather denote the presence of at least one of the referenced item. The term “or” means “and/or”. Recitation of ranges of values merely intend to serve as a shorthand method of referring individually to each separate value falling within the range, unless otherwise indicated herein, and each separate value is incorporated into the specification as if it were individually recited herein. The endpoints of all ranges are included within the range and independently combinable.

All processes described herein can be performed in a suitable order unless otherwise indicated herein or otherwise clearly contradicted by context. The use of example, or exemplary language (e.g., “such as”), is intended merely to better illustrate the invention and does not pose a limitation on the scope of the invention unless otherwise claimed.

Throughout the present application, the R/S system of nomenclature of enantiomers is followed. A chiral center having regard to the phosphorus atom P is labeled R_{P} or S_{P} according to a system in which the substituents on the atom P are each assigned a priority based on atomic number, according to the Cahn-Ingold-Prelog priority rules (CIP). Reference concerning the CIP rules is made to “Advanced Organic Chemistry” by J. March published by John Wiley & Sons (2007) and IUPAC Rules for the Nomenclature of Organic Chemistry, Section E, Stereochemistry (1974). The CIP rules allocate the lowest priority to the direct substituent on the chiral center P having the lowest atomic number. In the case of a phosphoramidate, this substituent is N. The P center is then orientated so that the N substituent is pointed away from the viewer. The atoms or next nearest atoms, if present, to the three O atoms directly linked to P are then considered, according to the CIP rules. If these atoms decrease in atomic number when viewed in a clockwise direction, the enantiomer is labeled RP. If these atoms decrease in atomic number in a counterclockwise direction, the enantiomer is labeled SP.

The symbol

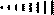

(dashed bond) present in some of the formulas of the specification and claims indicates that the substituent is directed below the plane of the sheet. The symbol

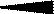

(wedge bond) present in some of the formulas of the specification and claims indicates that the substituent is directed above the plane of the sheet.

The compounds prepared by the processes of the present invention have one or more stereocenters, and may exist, be used or be isolated in diastereoisomerically pure forms or as diastereomeric enriched mixtures. It should be understood that the processes of the present invention may yield diastereoisomerically pure forms or diastereomeric enriched mixtures. It should also be understood that the products of the present invention may be isolated as diastereoisomerically pure forms or as diastereomeric enriched mixtures.

A diastereomeric mixture may contain the two diastereoisomers in any mutual ratio, unless otherwise indicated.

“Diastereomerically enriched” as used in the present application means that one of the diastereoisomers is present in excess of the other diastereoisomer.

“Diastereomerically pure” refers to a compound whose diastereoisomeric purity is at least about 90%, about 95%, or even about 99% or greater, and may be 100% pure.

“Alkyl” is a branched or straight chain saturated aliphatic hydrocarbon group. In one non-limiting embodiment, the alkyl group contains from about 1 to about 6 carbon atoms, more generally from 1 to about 4 carbon atoms, or from 1 to about 3 carbon atoms. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentance, 3-methylpentane, 2,2-dimethylbutane, and 2,3-dimethylbutane.

“Cycloalkyl” is a saturated group containing all carbon rings and from 3 to 6 carbon atoms (“C₃-C₆cycloalkyl”) and zero heteroatoms in a monocyclic or polycyclic (e.g. bicyclic or tricyclic) non-aromatic ring system. Non-limiting examples of “cycloalkyl” include: cyclopropyl, cyclobutyl, cyclopentyl, and cyclohexyl.

Any compound used in or formed by the processes described herein may be modified by making an inorganic or organic acid or base addition salt thereof to form a “pharmaceutically acceptable salt”, if appropriate under the conditions of use. The salts of the present compounds can be synthesized from a parent compound that contains a basic or acidic moiety by conventional chemical processes. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds may optionally be provided in the form of a solvate.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from inorganic or organic acids that are not unduly toxic. For example, conventional acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n—COOH where n is 0-4, and the like, or using a different acid that produces the same counterion. Lists of additional suitable salts may be found, e.g., in Remington’s Pharmaceutical Sciences, 17th ed., Mack Publishing Company, Easton, Pa., p. 1418 (1985).

The C₁ to C₈ alcohol refers to a straight/branched and/or cyclic/acyclic alcohol having any of the number of carbons within the range, and the range is specifically intended to independently disclose each compound within the range. The C₁ to C₈ alcohol includes, but is not limited to, methanol, ethanol, n-propanol, isopropanol, isobutanol, hexanol, and cyclohexanol.

The C₂ to C₈ ether refers to a straight/branched and/or cyclic/acyclic ether having any of the number of carbons within the range, and the range is specifically intended to independently disclose each compound within the range. The C₂ to C₈ ether includes, but is not limited to, dimethyl ether, diethyl ether, di-isopropyl ether, di-n-butyl ether, methyl-t-butyl ether (MTBE), tetrahydrofuran, and dioxane

The C₃ to C₇ ketone refers to a straight/branched and/or cyclic/acyclic ketone having any of the number of carbons within the range, and the range is specifically intended to independently disclose each compound within the range. The C₃ to C₇ ketone includes, but is not limited to, acetone, methyl ethyl ketone, propanone, butanone, methyl isobutyl ketone, methyl butyl ketone, and cyclohexanone.

The C₃ to C₇ ester refers to a straight/branched and/or cyclic/acyclic ester having any of the number of carbons within the range, and the range is specifically intended to independently disclose each compound within the range. The C₃ to C₇ ester includes, but is not limited to, ethyl acetate, propyl acetate, n-butyl acetate, etc.

The C₁ to C₂ chlorocarbon refers to a chlorocarbon with 1 or 2 carbons, with any number of chloro atoms that fulfill the desired purpose. The C₁ to C₂ chlorocarbon includes, but is not limited to, chloroform, methylene chloride (DCM), carbon tetrachloride, 1,2-dichloroethane, and tetrachloroethane.

A C₂ to C₇ nitrile refers to a nitrile having any of the number of carbons within the range, and the range is specifically intended to independently disclose each compound within the range. The C₂ to C₇ nitrile includes, but is not limited to, acetonitrile, propionitrile, etc.

A miscellaneous solvent refers to a solvent known to those skilled in the art and employed in organic chemistry, which includes, but is not limited to, diethylene glycol, diglyme (diethylene glycol dimethyl ether), 1,2-dimethoxy-ethane, dimethylformamide, dimethylsulfoxide, ethylene glycol, glycerin, hexamethylphsphoramide, hexamethylphosphorous triame, N-methyl-2-pyrrolidinone, nitromethane, pyridine, triethyl amine, and acetic acid.

The term C₅ to C₁₂ saturated hydrocarbon refers to a straight/branched and/or cyclic/acyclic hydrocarbon having any of the number of carbons within the range, and the range is specifically intended to independently disclose each compound within the range.. The C₅ to C₁₂ saturated hydrocarbon includes, but is not limited to, pentane (including n-pentane), petroleum ether (ligroine), hexane (including n-hexane), heptane (including n-heptane), cyclohexane, and cycloheptane.

The term C₆ to C₁₂ aromatic refers to a substituted and unsubstituted hydrocarbon having a phenyl group in its backbone. Examples of hydrocarbons include benzene, xylene, toluene, chlorobenzene, o-xylene, m-xylene, p-xylene, xylenes, with toluene being particularly useful.

### Compounds of Formula IIA, Formula IIIA, Formula II′, and Formula III′

The present invention also provides compounds of Formula IIA, Formula IIIA, Formula II′, and Formula III′:

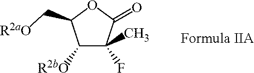

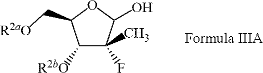

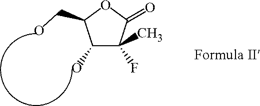

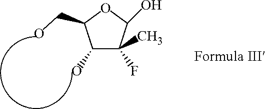

or a pharmaceutically acceptable salt thereof wherein
R^{2a} and R^{2b} are oxygen protecting groups and at least one of R^{2a} and R^{2b} is —C(O)OC₁-₆alkyl (for example —C(O)OtBu or —C(O)OCH₃), or —C(O)O-benzyl or in an alternative embodiment, at least one of R^{2a} and R^{2b} is —C(O)OC₁₋₂₀alkyl, —C(O)OC₂₋₂₀alkenyl, or —C(O)NR^{10a}R^{10b} wherein R^{10a} and R^{10b} are independently selected from hydrogen, C₁₋₂₀alkyl, C₂₋₂₀alkenyl, aryl, arylalkyl, heteroaryl, and heteroarylalkyl wherein R^{2a} and R^{2b} can independently be optionally substituted with a substituent selected from alkoxy, hydroxy, nitro, bromo, chloro, fluoro, azido, and haloalkyl; and
wherein the bridge structure in Formula II′ and Formula III′ is selected from

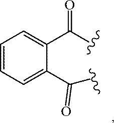

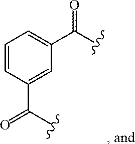

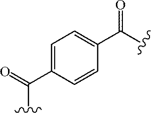

wherein the phenyl group of the bridge structure can be substituted with substituents selected from alkoxy (including but not limited to methoxy and ethoxy), hydroxy, nitro, bromo, chloro, fluoro, azido, and haloalkyl.

In an alternative embodiment, the bridge structure is selected from

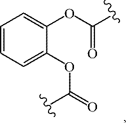

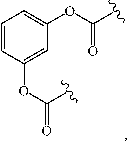

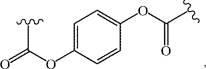

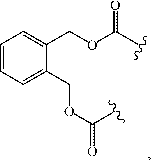

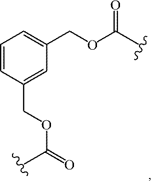

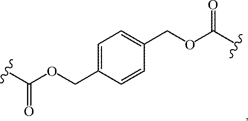

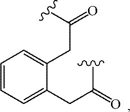

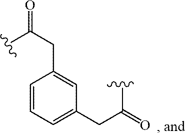

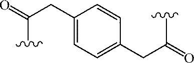

In one embodiment, a compound of Formula IIIA is of the Formula:

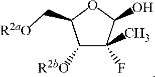

In one embodiment, a compound of Formula IIIA is of the Formula:

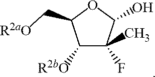

In one embodiment, a compound of Formula III′ is of the Formula:

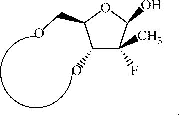

In one embodiment, a compound of Formula III′ is of the Formula:

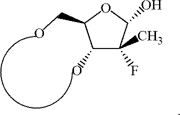

In one embodiment, R^{2a} and R^{2b} are both —C(O)OC₁₋₆alkyl, for example —C(O)OtBu. In one embodiment, R^{2a} and R^{2b} are both —C(O)O-benzyl. In one embodiment, R^{2a} is —C(O)OC₁₋₆alkyl or —C(O)O-benzyl and R^{2b} is an oxygen protecting group which when attached to the oxygen is an ester, ether, or silyl ether moiety. In an alternative embodiment, R^{2b} is —C(O)OC₁₋₆alkyl or —C(O)O-benzyl and R^{2a} is an oxygen protecting group which when attached to the oxygen is an ester, ether, or silyl ether moiety.

In an alternative embodiment, R^{2a} and R^{2b} are both —C(O)OCH₃. In an alternative embodiment, R^{2a} is —C(O)OCH₃ and R^{2b} is an oxygen protecting group which when attached to the oxygen is an ester, ether, or silyl ether moiety. In an alternative embodiment, R^{2b} is —C(O)OCH₃ and R^{2a} is an oxygen protecting group which when attached to the oxygen is an ester, ether, or silyl ether moiety.

In an alternative embodiment, at least one of R^{2a} and R^{2b} are selected from —C(O)OC₁₋₂₀alkyl, including —C(O)OC₁₋₁₈alkyl, —C(O)OC₁₋₁₆alkyl, —C(O)OC₁₋₁₄alkyl, —C(O)OC₁₋₁₂alkyl, —C(O)OC₁₋₁₀alkyl, —C(O)OC₁₋₈alkyl, —C(O)OC₁₋₆alkyl, —C(O)OC₁₋₄alkyl, —C(O)OC₁₋₂alkyl, —C(O)OC₂₋₂₀alkyl, —C(O)OC₂₋₂₀alkyl, —C(O)OC₂₋₂₀alkyl, —C(O)OC₈₋₂₀alkyl, —C(O)OC₁₀₋₂₀alkyl, —C(O)OC₁₂₋₂₀alkyl, —C(O)OC₁₄₋₂₀alkyl, —C(O)OC₁₆₋₂₀alkyl, and —C(O)OC₁₈₋₂₀alkyl. In one embodiment, both R^{2a} and R^{2b} are —C(O)OC₁₋₂₀alkyl. In one embodiment, both R^{2a} and R^{2b} are —C(O)OC₁₆H₃₃.

In an alternative embodiment, R^{2a} and R^{2b} are both —C(O)NR^{10a}R^{10b}, for example —C(O)NHPh, —C(O)NHBn, —C(O)N(Ph)₂, —C(O)N(Bn)₂, —C(O)NHC₁₋₂₀alkyl (including, but not limited to, —C(O)NHCH₃, —C(O)NHtBu, and —C(O)NHC₁₆H₃₃), and —C(O)N(C₁₋₂₀alkyl)₂ including, but not limited to, —C(O)N(CH₃)₂, —C(O)N(tBu)₂, and —C(O)N(C₁₆H₃₃)₂). In one embodiment, R^{2a} and R^{2b} are both —C(O)NR^{10a}R^{10b}. In one embodiment, R^{2a} is —C(O)NR^{10a}R^{10b} and R^{2b} is an oxygen protecting group which when attached to the oxygen is an ester, ether, or silyl ether moiety. In an alternative embodiment, R^{2b} is —C(O)NR^{10a}R^{10b} and R^{2a} is an oxygen protecting group which when attached to the oxygen is an ester, ether, or silyl ether moiety.

In one embodiment, at least one of R^{2a} and R^{2b} are —C(O)NHC₁₋₂₀alkyl, including —C(O)NHC₁₋₁₈alkyl, —C(O)NHC₁₋₁₆alkyl, —C(O)NHC₁₋₁₄alkyl, —C(O)NHC₁₋₁₂alkyl, —C(O)NHC₁₋alkyl, —C(O)NHC₁₋₈alkyl, —C(O)NHC₁₋₆alkyl, —C(O)NHC₁₋₄alkyl, —C(O)NHC₁₋₂alkyl, —C(O)NHC₂₋₂₀alkyl, —C(O)NHC₄₋₂₀alkyl, —C(O)NHC₆₋₂₀alkyl, —C(O)NHC₈₋₂₀alkyl, —C(O)NHC₁₀₋₂₀alkyl, —C(O)NHC₁₂₋₂₀alkyl, —C(O)NHC₁₄₋₂₀alkyl, —C(O)NHC₁₆₋₂₀alkyl, and —C(O)NHC₁₈₋₂₀alkyl. In one embodiment, both R^{2a} and R^{2b} are —C(O)NHC₁₋₂₀alkyl. In one embodiment, both R^{2a} and R^{2b} are —C(O)NHC₁₆H₃₃.

In one embodiment, the protecting group that when attached to the oxygen is an ester moiety, for example benzoate acetate. In one embodiment, the oxygen protecting group that when attached to the oxygen is a silyl ether moiety (for example (trimethylsilyl (TMS), triisopropylsilyl (TIPS), tert-butyldimethylsilyl (TBDMS or TBS) or tert-butyldiphenylsilyl (TBDPS). In one embodiment, the oxygen protecting group that when attached to the oxygen is an ether moiety, for example methyl ether, methoxymethyl ether, or benzyl ether. These protecting groups can be installed according to one of the procedures described in Theodora W. Green, Protective Groups in Organic Synthesis, Third Edition, John Wiley & Sons (1999), which is incorporated by reference, for the protection of hydroxyls. For example, when the oxygen protecting group which when attached to the oxygen is an ester moiety, the compound of Formula II, Formula IIA, Formula III, Formula IIIA, Formula II′, or Formula III′ can be prepared according to the conditions described in the text on page 149-178 and when the oxygen protecting group is a silyl ether moiety when attached to the oxygen, the compound of Formula II, Formula IIA, Formula III, Formula IIIA, Formula II′, or Formula III′ can be prepared according to the conditions described in the text on page 113-147. In one embodiment, the protecting group is a tert-butyldimethylsilyl (TBS) group. The TBS group is selectively installed on the primary alcohol over the secondary alcohol using the conditions described in the text on page 128 and in Ogilvie et al. Can. J. Chem. 1979, 57, 2230. These conditions include the use of TBSCl, DMAP, and NEt₃ in DMF at 25° C.

Non-limiting examples of additional protecting groups which when attached to the oxygen also include bromobenzoate,p-methoxybenzyloxymethyl ether (MPBM), o-nitrobenzyloxymethyl ether (NBOM), p-nitrobenzyloxymethyl ether, t-butoxymethyl ether, 2,2,2-trichloroethoxymethyl ether, 3-bromotetrahydropyranyl ether, tetrahydropyranyl ether, tetrahydrothiopyranyl ether, 1-methoxycyclohexyl ether, 1,4-dioxan-2-yl ether, tetrahydrofuranyl ether, tetrahydrothiofuranyl ether, a substituted phenyl ether, 2-picolyl ether, 4-picolyl ether, 1,3-benzodithiolan-2-yl ether, p-chlorophenoxyacetate ester, 3-phenylpropionate ester, p-phenylbenzoate ester, alkyl p-nitrophenyl carbonyl, alkyl benzyl carbonyl, alkyl p-methoxybenzyl carbonyl, alkyl o-nitrobenzyl carbonyl, and alkyl p-nitrobenzyl carbonyl.

Non-limiting examples of R^{2a} and R^{2b} include:

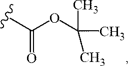

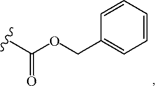

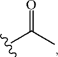

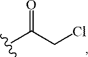

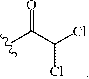

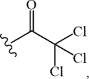

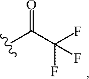

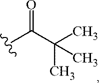

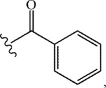

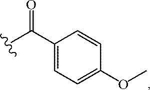

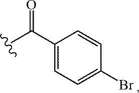

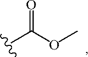

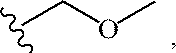

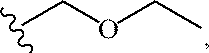

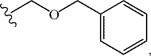

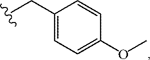

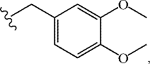

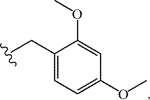

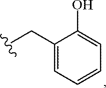

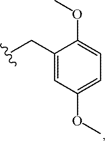

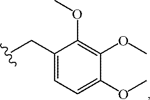

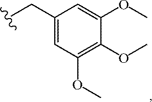

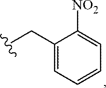

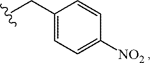

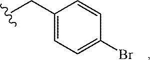

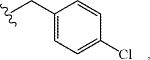

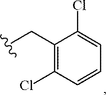

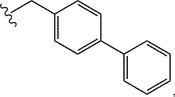

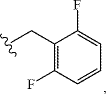

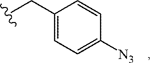

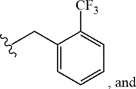

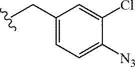

Additional non-limiting examples of R^{2a} and R^{2b} include:

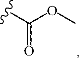

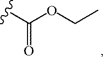

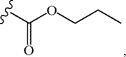

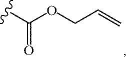

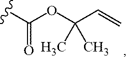

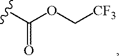

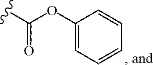

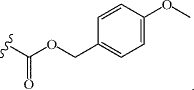

Additional non-limiting examples of R^{2a} and R^{2b} include:

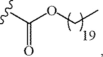

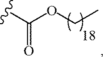

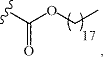

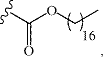

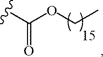

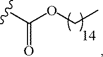

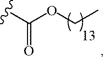

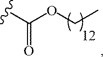

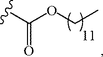

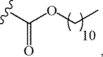

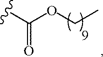

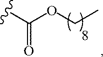

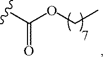

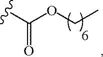

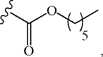

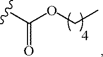

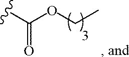

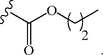

Additional non-limiting examples of R^{2a} and R^{2b} include:

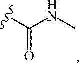

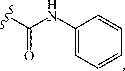

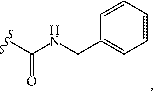

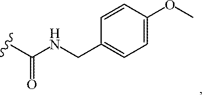

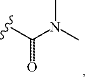

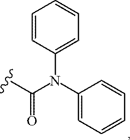

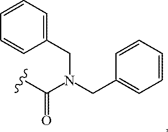

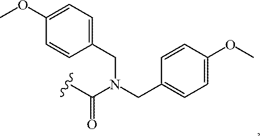

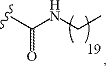

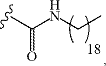

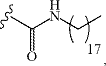

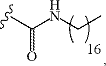

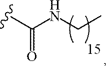

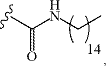

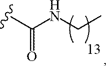

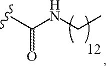

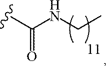

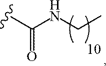

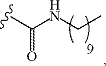

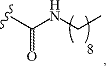

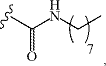

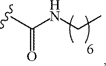

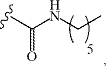

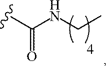

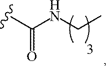

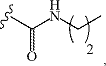

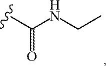

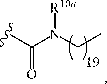

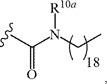

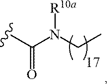

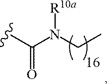

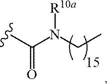

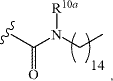

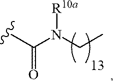

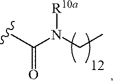

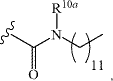

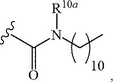

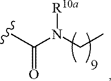

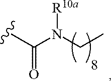

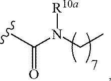

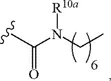

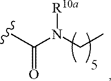

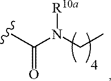

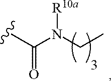

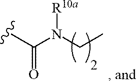

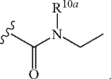

In one embodiment, R^{10a} is hydrogen. In one embodiment, R^{10a} is phenyl.

In one embodiment, a carbonate or carbamate compound of Formula IIIA is of the Formula:

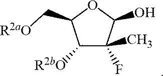

In one embodiment, a carbonate or carbamate compound of Formula IIIA is of the Formula:

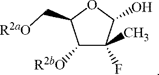

In one embodiment, a bridged compound of Formula III′ is of the Formula:

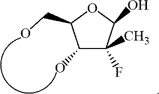

In one embodiment, a bridged compound of Formula III′ is of the Formula:

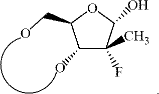

Non-limiting examples of a carbonate or carbamate compound of Formula IIA and Formula IIIA or bridged compound of Formula II′ and Formula III′ include:

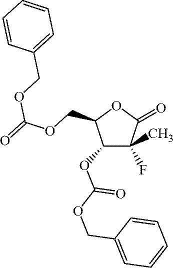 [SMILES: C[C@]1(F)C(=O)O[C@H](COC(=O)OCc2ccccc2)[C@H]1OC(=O)OCc1ccccc1]

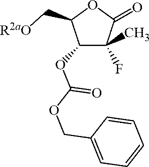

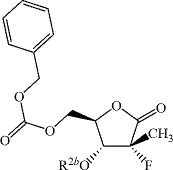

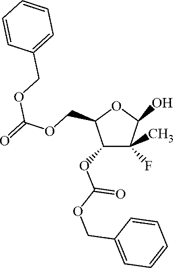 [SMILES: C[C@]1(F)[C@H](O)O[C@H](COC(=O)OCc2ccccc2)[C@H]1OC(=O)OCc1ccccc1]

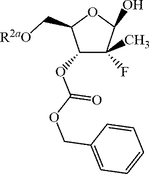

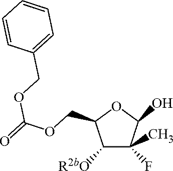

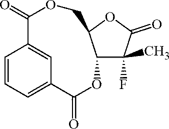 [SMILES: C[C@]1(F)C(=O)O[C@@H]2COC(=O)c3cccc(c3)C(=O)O[C@H]21]

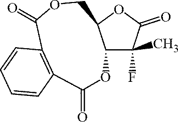 [SMILES: C[C@]1(F)C(=O)O[C@@H]2COC(=O)c3ccccc3C(=O)O[C@H]21]

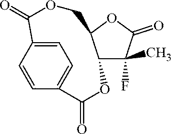 [SMILES: C[C@]1(F)C(=O)O[C@@H]2COC(=O)c3ccc(cc3)C(=O)O[C@H]21]

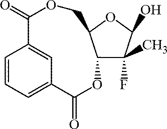 [SMILES: C[C@]1(F)[C@H](O)O[C@@H]2COC(=O)c3cccc(c3)C(=O)O[C@H]21]

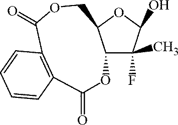 [SMILES: C[C@]1(F)[C@H](O)O[C@@H]2COC(=O)c3ccccc3C(=O)O[C@H]21]

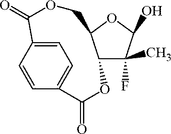 [SMILES: C[C@]1(F)[C@H](O)O[C@@H]2COC(=O)c3ccc(cc3)C(=O)O[C@H]21]

Additional non-limiting examples of a carbonate compound of Formula IIA include:

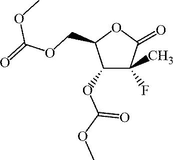 [SMILES: COC(=O)OC[C@H]1OC(=O)[C@](C)(F)[C@@H]1OC(=O)OC]

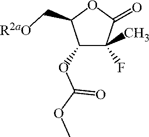

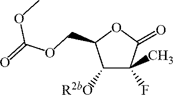

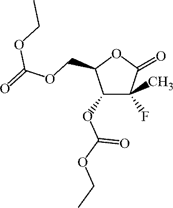 [SMILES: CCOC(=O)OC[C@H]1OC(=O)[C@](C)(F)[C@@H]1OC(=O)OCC]

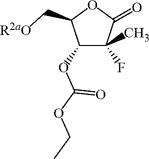

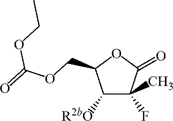

Additional non-limiting examples of a carbonate or carbamate compound of Formula IIA and Formula IIIA include:

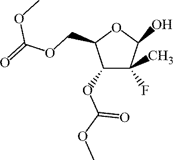 [SMILES: COC(=O)OC[C@H]1O[C@@H](O)[C@](C)(F)[C@@H]1OC(=O)OC]

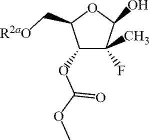

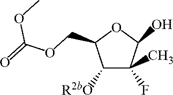

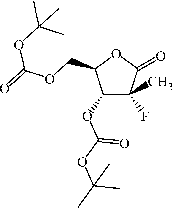 [SMILES: CC(C)(C)OC(=O)OC[C@H]1OC(=O)[C@](C)(F)[C@@H]1OC(=O)OC(C)(C)C]

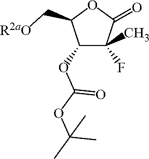

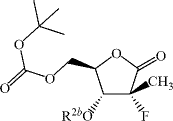

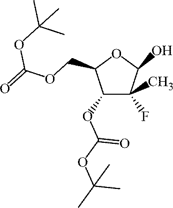 [SMILES: CC(C)(C)OC(=O)OC[C@H]1O[C@@H](O)[C@](C)(F)[C@@H]1OC(=O)OC(C)(C)C]

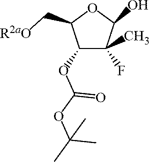

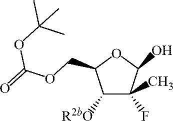

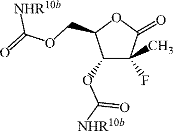

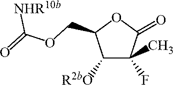

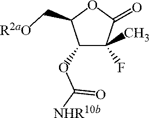

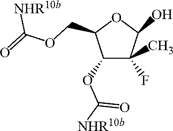

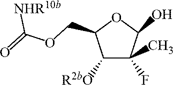

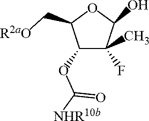

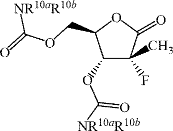

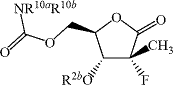

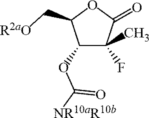

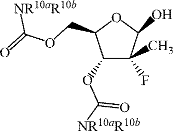

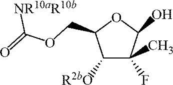

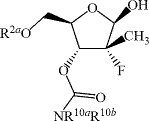

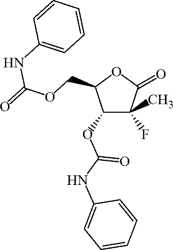 [SMILES: C[C@]1(F)C(=O)O[C@H](COC(=O)Nc2ccccc2)[C@H]1OC(=O)Nc1ccccc1]

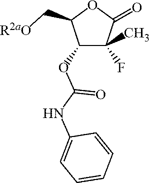

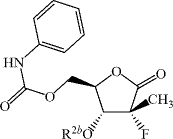

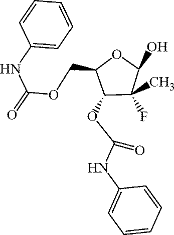 [SMILES: C[C@]1(F)[C@H](O)O[C@H](COC(=O)Nc2ccccc2)[C@H]1OC(=O)Nc1ccccc1]

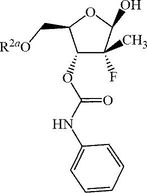

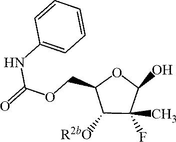

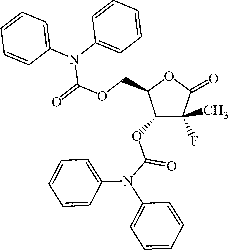 [SMILES: C[C@]1(F)C(=O)O[C@H](COC(=O)N(c2ccccc2)c2ccccc2)[C@H]1OC(=O)N(c1ccccc1)c1ccccc1]

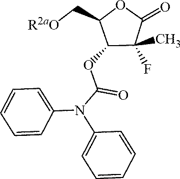

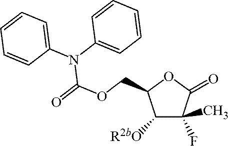

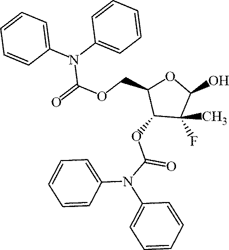 [SMILES: C[C@]1(F)[C@H](O)O[C@H](COC(=O)N(c2ccccc2)c2ccccc2)[C@H]1OC(=O)N(c1ccccc1)c1ccccc1]

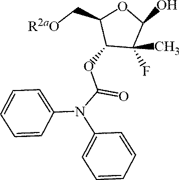

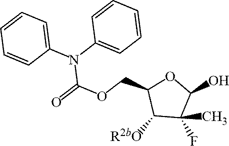

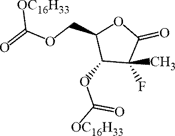

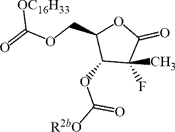

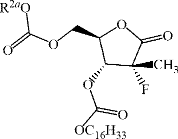

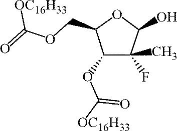

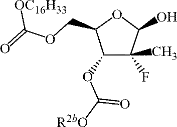

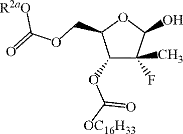

Non-limiting examples of a carbonate compound of Formula IIA and IIIA include:

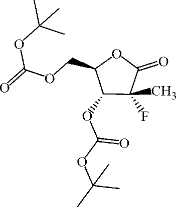 [SMILES: CC(C)(C)OC(=O)OC[C@H]1OC(=O)[C@](C)(F)[C@@H]1OC(=O)OC(C)(C)C]

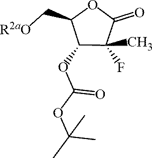

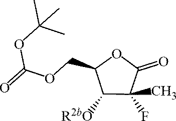

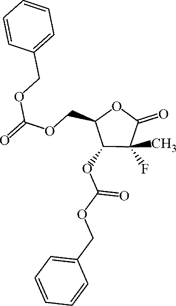 [SMILES: C[C@]1(F)C(=O)O[C@H](COC(=O)OCc2ccccc2)[C@H]1OC(=O)OCc1ccccc1]

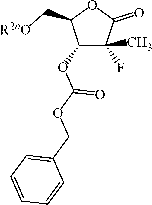

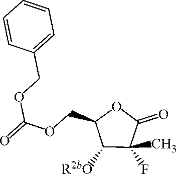

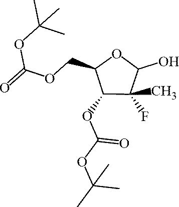 [SMILES: CC(C)(C)OC(=O)OC[C@H]1OC(O)[C@](C)(F)[C@@H]1OC(=O)OC(C)(C)C]

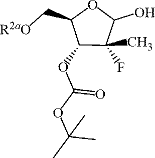

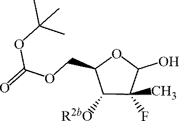

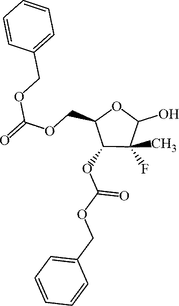 [SMILES: C[C@]1(F)C(O)O[C@H](COC(=O)OCc2ccccc2)[C@H]1OC(=O)OCc1ccccc1]

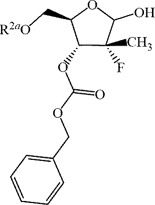

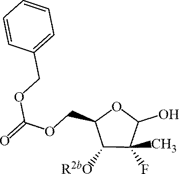

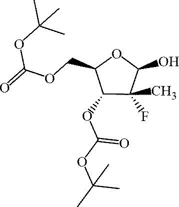 [SMILES: CC(C)(C)OC(=O)OC[C@H]1O[C@@H](O)[C@](C)(F)[C@@H]1OC(=O)OC(C)(C)C]

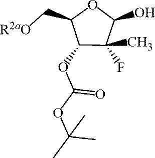

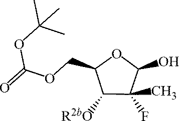

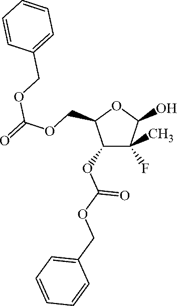 [SMILES: C[C@]1(F)[C@H](O)O[C@H](COC(=O)OCc2ccccc2)[C@H]1OC(=O)OCc1ccccc1]

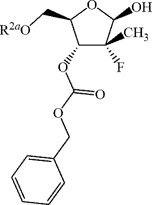

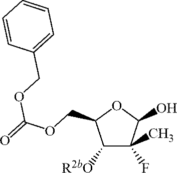

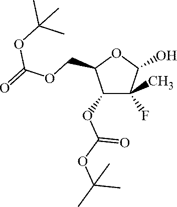 [SMILES: CC(C)(C)OC(=O)OC[C@H]1O[C@H](O)[C@](C)(F)[C@@H]1OC(=O)OC(C)(C)C]

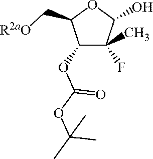

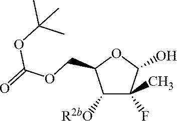

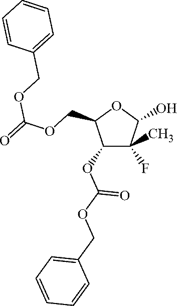 [SMILES: C[C@@]1(F)[C@H](OC(=O)OCc2ccccc2)[C@@H](COC(=O)OCc2ccccc2)O[C@@H]1O]

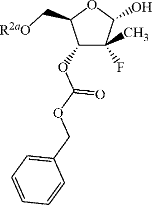

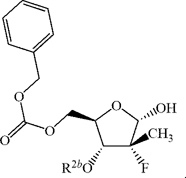

Additional non-limiting examples of a carbamate compound of Formula IIA and IIIA include:

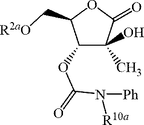

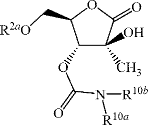

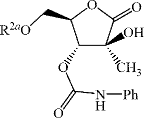

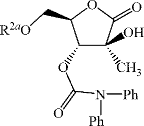

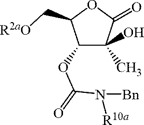

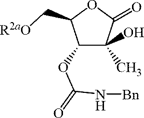

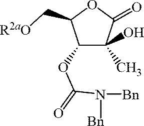

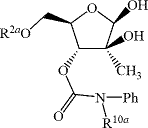

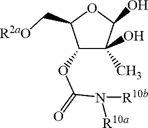

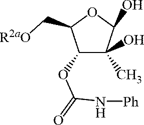

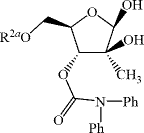

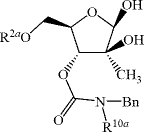

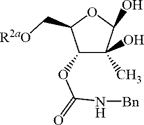

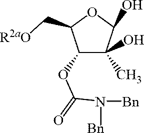

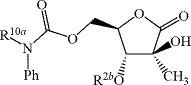

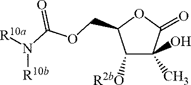

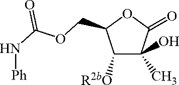

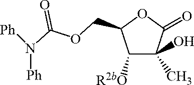

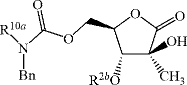

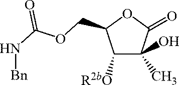

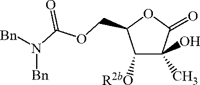

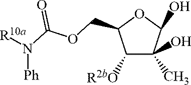

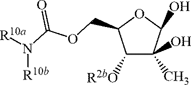

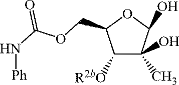

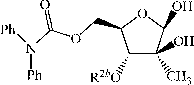

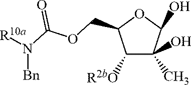

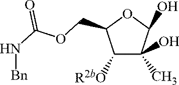

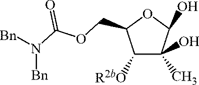

Additional non-limiting examples of a carbonate compound of Formula II′ and III′ include:

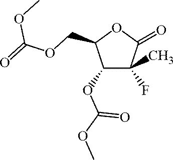 [SMILES: COC(=O)OC[C@H]1OC(=O)[C@](C)(F)[C@@H]1OC(=O)OC]

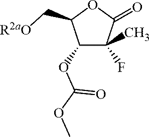

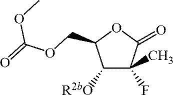

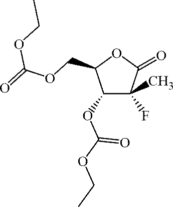 [SMILES: CCOC(=O)OC[C@H]1OC(=O)[C@](C)(F)[C@@H]1OC(=O)OCC]

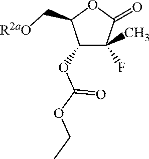

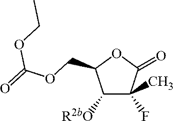

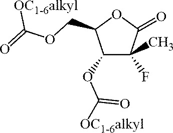

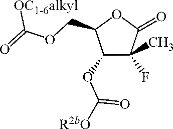

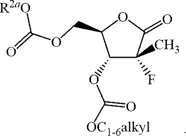

Additional non-limiting examples of a bridged compound of Formula II′ and III′ include:

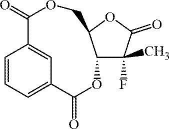 [SMILES: C[C@]1(F)C(=O)O[C@@H]2COC(=O)c3cccc(c3)C(=O)O[C@H]21]

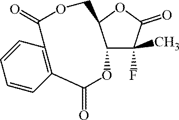 [SMILES: C[C@]1(F)C(=O)O[C@@H]2COC(=O)c3ccccc3C(=O)O[C@H]21]

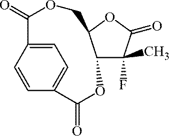 [SMILES: C[C@]1(F)C(=O)O[C@@H]2COC(=O)c3ccc(cc3)C(=O)O[C@H]21]

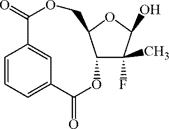 [SMILES: C[C@]1(F)[C@H](O)O[C@@H]2COC(=O)c3cccc(c3)C(=O)O[C@H]21]

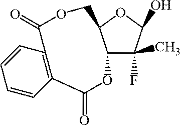 [SMILES: C[C@]1(F)[C@H](O)O[C@@H]2COC(=O)c3ccccc3C(=O)O[C@H]21]

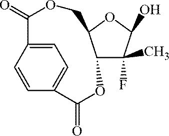 [SMILES: C[C@]1(F)[C@H](O)O[C@@H]2COC(=O)c3ccc(cc3)C(=O)O[C@H]21]

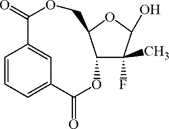 [SMILES: C[C@]1(F)C(O)O[C@@H]2COC(=O)c3cccc(c3)C(=O)O[C@H]21]

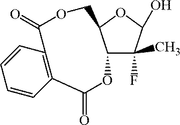 [SMILES: C[C@]1(F)C(O)O[C@@H]2COC(=O)c3ccccc3C(=O)O[C@H]21]

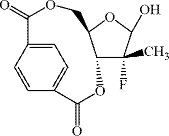 [SMILES: C[C@]1(F)C(O)O[C@@H]2COC(=O)c3ccc(cc3)C(=O)O[C@H]21]

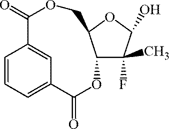 [SMILES: C[C@@]1(F)[C@@H]2OC(=O)c3cccc(c3)C(=O)OC[C@H]2O[C@@H]1O]

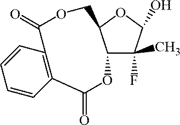 [SMILES: C[C@@]1(F)[C@@H]2OC(=O)c3ccccc3C(=O)OC[C@H]2O[C@@H]1O]

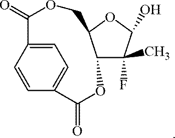

### Additional Embodiments

1. In certain embodiments a process is provided for preparing a diastereomer Sₚ-phosphoramidate nucleotide of Formula XVI, wherein the nucleotide of Formula XVI is greater than about 90% pure, comprising the steps of contacting the nucleoside Compound 2 with a compound of Formula XVII dihydroquinine salt, and an activator and a base to afford the diastereomer Sₚ-phosphoramidate nucleotide of Formula XVI:

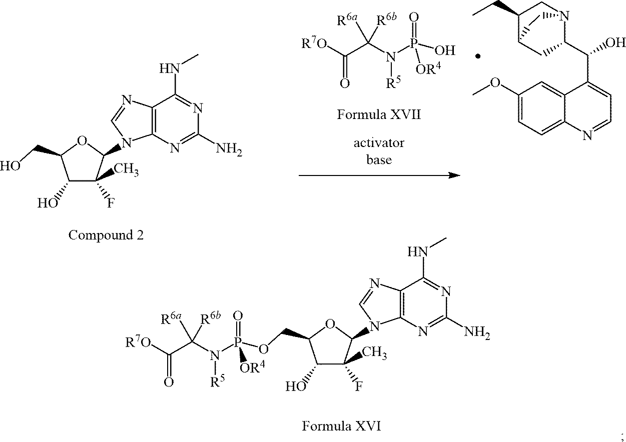

(b) optionally further purifying the diastereomerically enriched Sₚ-phosphoramidate nucleotide of Formula XVI to increase the purity; wherein:
   R⁴ is hydrogen, C₁₋₆alkyl, C₃₋₇cycloalkyl, or aryl;
   R⁵ is hydrogen or C₁₋₆alkyl;
   R^{6a} and R^{6b} are independently selected from the group consisting of hydrogen, C₁₋₆alkyl, and C₃₋₇cycloalkyl; and
   R⁷ is hydrogen, C₁₋₆alkyl, C₁₋₆haloalkyl, or C₃₋₇cycloalkyl.

2. The process of embodiment 1 wherein R⁴ is aryl.

3. The process of embodiment 1 or 2 wherein R⁵ is hydrogen.

4. The process of any one of embodiments 1-3 wherein at least one of R^{6a} and R^{6b} is hydrogen.

5. The process of any one of embodiments 1-4 wherein R^{6a} and R^{6b} are hydrogen and methyl.

6. The process of any one of embodiments 1-5 wherein R⁷ is C₁₋₆alkyl.

7. The process of any one of embodiments 1-6 wherein the activator is selected from HOBt ((1-hydroxybenzotriazole), PyBOP (benzotriazol-1-yloxytri(pyrrolidino)phosphonium hexafluorophosphate), HATU (O-(7-azabenzotriazol-1-yl)-1,1,3,3-tetramethyluronium hexafluorophosphate), HBTU (3-[bis(dimethylamino)methyliumyl]-3H-benzotriazol-1-oxide hexafluorophosphate), HCTU (2-(6-Chloro-1H-benzotriazole-1-yl)-1,1,3,3-tetramethylaminium hexafluorophosphate), COMU ((1-Cyano-2-ethoxy-2-oxoethylidenaminooxy)dimethylamino-morpholino-carbenium hexafluorophosphate), and TBTU (O-benzotriazol-1-yl-1,1,3,3-tetramethyluronium tetrafluoroborate).

8. The process of any one of embodiments 1-7 wherein the activator is HATU (O-(7-azabenzotriazol-1-yl)-1,1,3,3-tetramethyluronium hexafluorophosphate).

9. The process of any one of embodiments 1-7 wherein the activator is COMU ((1-Cyano-2-ethoxy-2-oxoethylidenaminooxy)dimethylamino-morpholino-carbenium hexafluorophosphate).

10. The process of any one of embodiments 1-9, wherein the base is NR₃ and R can be selected independently in each instance from H, alkyl, aryl, heteroaryl, alkenyl, alkynyl, benzyl and allyl, wherein at least one R is not hydrogen.

11. The process of any one of embodiments 1-10, wherein the base is DIPEA (N,N-diisopropylethylamine) or NEt₃ (triethylamine).

12. The process of any one of embodiments 1-11, wherein the base is DIPEA (N,N-diisopropylethylamine).

13. The process of any one of embodiments 1-9, wherein the base is quinine.

14. The process of any one of embodiments 1-13 wherein step (a) is performed in a polar aprotic solvent.

15. The process of embodiment 13 wherein the solvent is selected from dimethylformamide (DMF), dichloromethane (DCM), tetrahydrofuran (THF), 2-methyltetrahydrofuran (2-MeTHF), ethyl acetate (EtOAc), acetonitrile (MeCN), dimethyl sulfoxide (DMSO), acetone, and N-methylpyrrolidone.

16. The process of any one of embodiments 1-13 wherein step (a) is performed in a mixture of solvents selected from dimethylformamide (DMF), dichloromethane (DCM), tetrahydrofuran (THF), 2-methyltetrahydrofuran (2-MeTHF), ethyl acetate (EtOAc), acetonitrile (MeCN), dimethyl sulfoxide (DMSO), acetone, and N-methylpyrrolidone.

17. The process of step 16 wherein the mixture of solvents comprises dichloromethane (DCM) and 2-methyltetrahydrofuran (2-MeTHF).

18. The process of any one of embodiments 1-17, wherein the ratio of Sₚ:Rₚ diastereomers before step (b) is greater than about 60:40.

19. The process of any one of embodiments 1-18, wherein the ratio of Sₚ:Rₚ diastereomers before step (b) is greater than about 70:30.

20. The process of any one of embodiments 1-19, wherein the ratio of Sₚ:Rₚ diastereomers before step (b) is greater than about 80:20.

21. The process of any one of embodiments 1-20, wherein the ratio of Sₚ:Rₚ diastereomers before step (b) is greater than about 90:10.

22. The process of any one of embodiments 1-21, wherein the nucleotide of Formula XVI is greater than about 98% pure

23. The process of any one of embodiments 1-22, wherein the nucleotide of Formula XVI is greater than about 99% pure

24. The process of any one of embodiments 1-23, wherein the purification of step (b) is a selective crystallization.

25. The process of embodiment 24, wherein the crystallization is conducted in a polar organic solvent.

26. The process of embodiment 24 or 25, wherein the crystallization is conducted in an alkyl ester.

27. The process of any one of embodiments 24-26, wherein the crystallization is conducted in ethyl acetate or isopropyl acetate.

28. The process of embodiment 24, wherein the crystallization is conducted in a mixture of solvents.

29. The process of embodiment 24 or 28 wherein the crystallization is conducted in a mixture of polar organic solvent and aromatic solvent.

30. The process of any one of embodiments 24, 28, or 29, wherein the crystallization is conducted in a mixture of ethyl acetate and toluene.

31. The process of any one of embodiments 1-30, further comprising step (c) wherein the compound of Formula XVI is converted to a pharmaceutically acceptable salt.

32. The process of embodiment 31, wherein the pharmaceutically acceptable salt is the hemi-sulfate salt.

33. The process of any one of embodiments 1-32 wherein the compound of Formula XVI has the structure

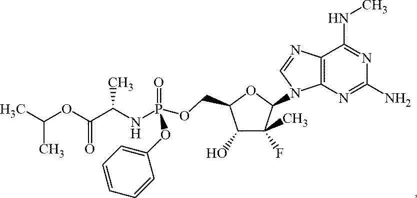

and the compound of Formula XVII has the structure

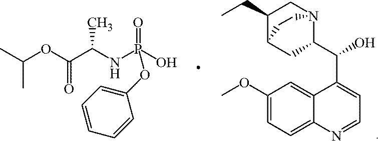

34. In certain embodiments a process is provided for preparing Compound 2 comprising the steps of:
(a) protecting (3S,4R,5R)-3,4-dihydroxy-5-(hydroxymethyl)-3-methyldihydrofuran-2(3H)-one at the 5′ and 3′ hydroxyl to afford a compound of Formula I

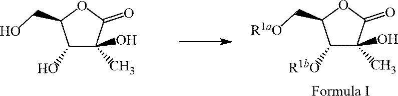

(b) converting the compound of Formula I into a compound of Formula II:

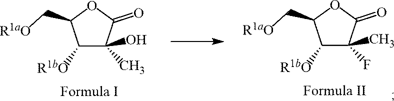

(c) reducing the compound of Formula II to afford a compound of Formula III:

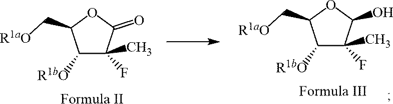

(d) converting the compound of Formula III to a compound of Formula IV

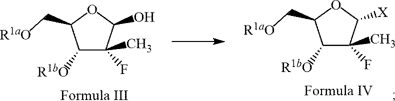

(e) converting the compound of Formula IV to a compound of Formula V via nucleophilic substitution with 2-amino-6-chloropurine:

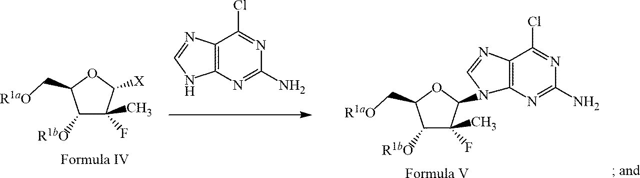

(f) converting the compound of Formula V to Compound 2:

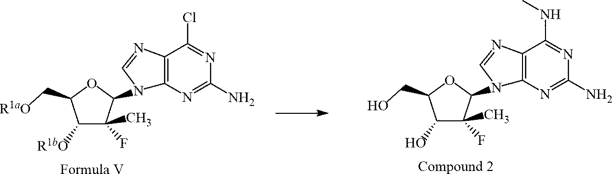

wherein:
   R^{1a} and R^{1b} are oxygen protecting groups;
   at least one of R^{1a} and R^{1b} is —C(O)OC₁₋₆alkyl, —C(O)O-benzyl, or —CH₂-phenyl wherein the phenyl group is substituted with at least one substituent selected from alkoxy, hydroxy, nitro, bromo, chloro, fluoro, azido, and haloalkyl;
   at least one of R^{1a} and R^{1b} is —C(O)OC₁₋₂₀alkyl, —C(O)OC₂₋₂₀alkenyl, or —C(O)NR₁₀ₐR_{10b};
   R^{10a} and R^{10b} are independently selected from hydrogen, C₁₋₂₀alkyl, C₂₋₂₀alkenyl, aryl, arylalkyl, heteroaryl, and heteroarylalkyl wherein the C₁₋₂₀alkyl, C₂₋₂₀alkenyl, aryl, arylalkyl, heteroaryl, and heteroarylalkyl can optionally be substituted with 1, 2, 3, or 4 substituents independently selected from alkoxy, hydroxy, nitro, bromo, chloro, fluoro, azido, and haloalkyl; and
   X is Cl, Br, or OAc.

35. The process of embodiment 34 wherein X is Br.

36. The process of embodiment 34 or 35 wherein R^{1a} and R^{1b} are —C(O)OtBu.

37. In certain embodiments a process is provided for preparing Compound 2 comprising the steps of:
(a) protecting (3S,4R,5R)-3,4-dihydroxy-5-(hydroxymethyl)-3-methyldihydrofuran-2(3H)-one at the 5′ and 3′ hydroxyl to form a bridge structure to afford a compound of Formula I′

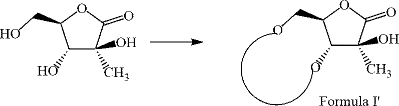

(b) converting the compound of Formula I′ into a compound of Formula II′:

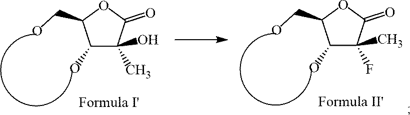

(c) reducing the compound of Formula II′ to afford a compound of Formula III′:

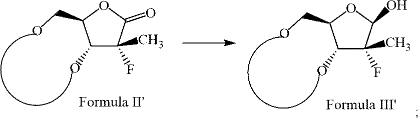

(d) converting the compound of Formula III′ to a compound of Formula IV′

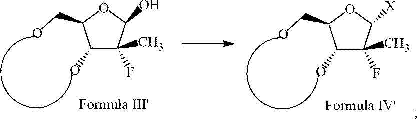

(e) converting the compound of Formula IV′ to a compound of Formula V′ via nucleophilic substitution with 2-amino-6-chloropurine:

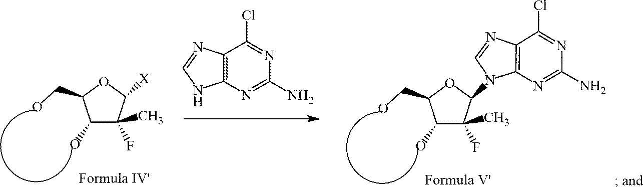

(f) converting the compound of Formula V′ to Compound 2:

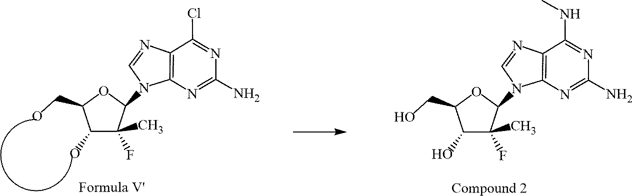

wherein:
   the bridge structure is selected from: 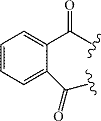 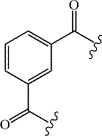 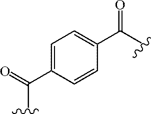 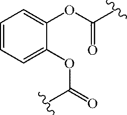 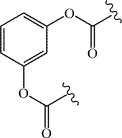 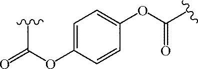 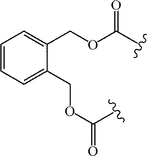 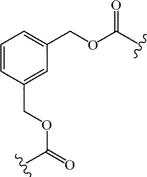 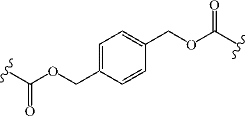 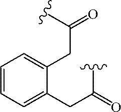 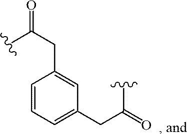 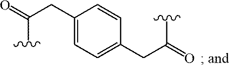
   X is Cl, Br, or OAc.

38. The process of any one of embodiments 34-37 wherein step (b) is performed with a fluorinating agent selected from a sulfonyl fluoride/TREAT•HF mixture (SO₂F₂, NEt₃·3HF), DAST (Et₂NSF₃), and morpholinosulfur trifluoride (Morph-DAST).

39. The process of any one of embodiments 34-38 wherein step (c) is performed with a reducing agent selected from DIBAL-H (diisobutylaluminium hydride), NaBH₄, Red-Al® (sodium bis(2-methoxyethoxy)aluminum hydride), and LiAlH₄ (lithium aluminum hydride).

40. The process of any one of embodiments 34-39 wherein step (d) is performed with triphenylphosphine and dibromohydantoin.

41. The process of any one of embodiments 34-40 wherein step (e) is performed with a non-nucleophilic base selected from sodium tert-butoxide, potassium tert-butoxide, sodium tert-pentoxide, potassium tert-pentoxide, lithium diisopropylamide, and lithium bis(trimethylsilyl)amide.

42. The process of any one of embodiments 34-41 wherein step (f) further comprises the steps:
(f.1) reacting the compound of Formula V or V′ with methylamine (NH₂Me) to afford a crude mixture; and
(f.2) reacting the crude mixture of step (f.1) under deprotection conditions to afford Compound 2.

43. The process of embodiment 42 wherein the deprotection conditions are DBU and methanol.

44. In certain embodiments a process is provided to prepare a compound of Formula VII comprising the steps:
(a) Converting a compound of Formula V to a compound of Formula VI

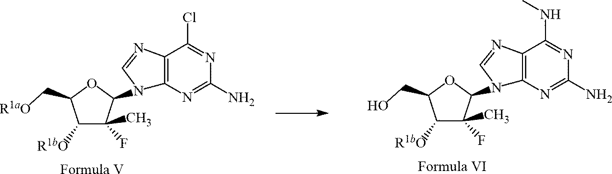

(b) Reacting the compound of Formula VI with the dihydroquinine salt of isopropyl (hydroxy(phenoxy)phosphoryl)-L-alaninate in the presence of an activator and a base to afford a protected diastereomerically enriched Sₚ-phosphoramidate nucleotide of Formula VII wherein the Sₚ-diastereomer is in excess of the Rₚ-diastereomer

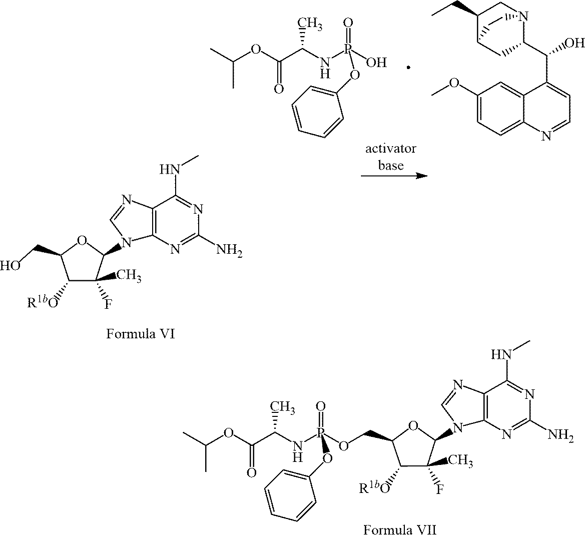

wherein:
   R^{1a} and R^{1b} are oxygen protecting groups;
   at least one of R^{1a} and R^{1b} is —C(O)OC₁₋₆alkyl, —C(O)O-benzyl, or —CH₂-phenyl wherein the phenyl group is substituted with at least one substituent selected from alkoxy, hydroxy, nitro, bromo, chloro, fluoro, azido, and haloalkyl;
   at least one of R^{1a} and R^{1b} is —C(O)OC₁₋₂₀alkyl, —C(O)OC₂₋₂₀alkenyl, or —C(O)NR^{10a}R^{10b}; and
   R^{10a} and R^{10b} are independently selected from hydrogen, C₁₋₂₀alkyl, C₂₋₂₀alkenyl, aryl, arylalkyl, heteroaryl, and heteroarylalkyl wherein the C₁₋₂₀alkyl, C₂₋₂₀alkenyl, aryl, arylalkyl, heteroaryl, and heteroarylalkyl can optionally be substituted with at least one substituent selected from alkoxy, hydroxy, nitro, bromo, chloro, fluoro, azido, and haloalkyl.

45. The process of embodiment 44 wherein step (a) is performed in a single transformation with methylamine.

46. The process of embodiment 44 or 45 wherein the activator in step (b) is HATU (O-(7-azabenzotriazol-1-yl)-1,1,3,3-tetramethyluronium hexafluorophosphate) or COMU ((1-cyano-2-ethoxy-2-oxoethylidenaminooxy)dimethylamino-morpholino-carbenium hexafluorophosphate).

47. The process of any one of embodiments 44-46 wherein the base in step (b) is DIPEA (diisopropylethylamime).

48. The process of any one of embodiments 44-47 further comprising step (c) wherein the compound of Formula VII is converted to Compound 1.

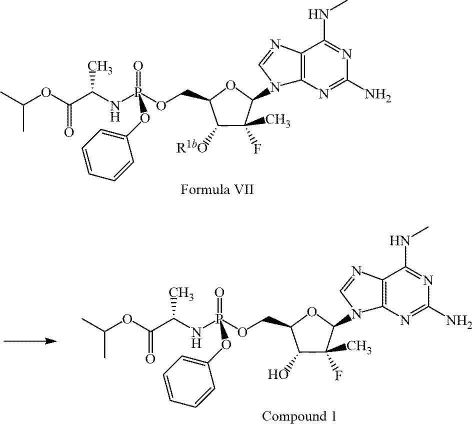

49. The process of embodiment 48 wherein step (c) is performed with DBU in methanol.

50. In certain embodiments a process is provided to prepare a compound of Formula IX by reacting a compound of Formula VIII with the dihydroquinine salt of isopropyl (hydroxy(phenoxy)phosphoryl)-L-alaninate with an activator and a base to afford a protected diastereomerically enriched Sₚ-phosphoramidate nucleotide of Formula IX wherein the Sₚ-diastereomer is in excess of the Rₚ-diastereomer:

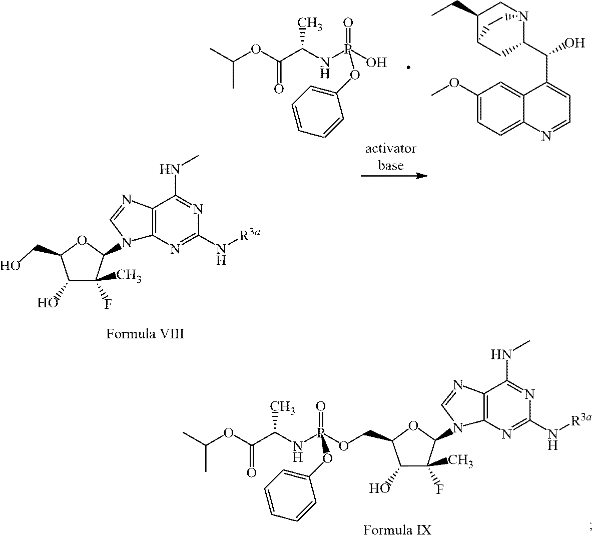

wherein:

R^{3a} is selected from tert-butoxycarbonyl-(Boc), benzyloxycarbonyl-(Cbz), benzyl, and p-methoxybenzyl.

51. The process of embodiment 50 further comprising preparing the compound of Formula VIII by the process consisting of the steps:
(a) Protecting a compound of Formula V at the N²-position to afford a compound of Formula X

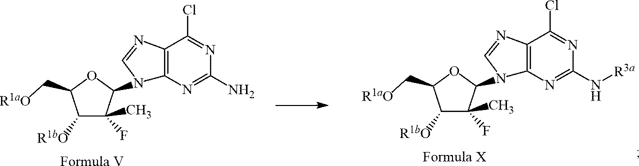

(b) Converting the compound of Formula X to the compound of Formula VIII

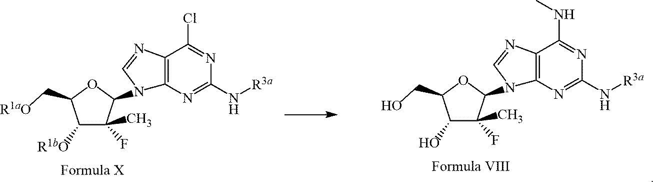

52. The process of embodiment 51 wherein the step (b) is performed in a single transformation with methylamine (NH₂Me).

53. The process of any one of embodiments 50-52 further comprising converting the compound of Formula IX to compound 1

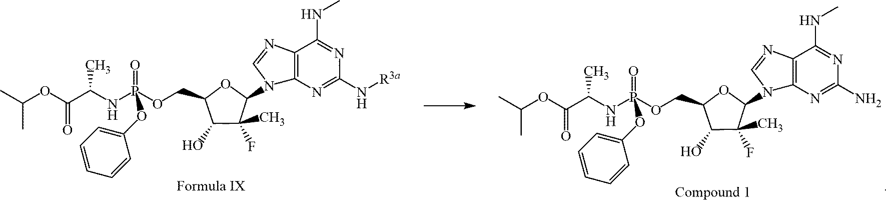

54. The process of embodiment 53 wherein the compound of Formula IX is converted to Compound 1 by reacting with Pd/C and hydrogen.

55. The process of embodiment 53 wherein the compound of Formula IX is converted to Compound 1 by reacting with HCl in DCM.

56. The process of any one of embodiments 50-55 wherein the activator is HATU (O-(7-azabenzotriazol-1-yl)-1,1,3,3-tetramethyluronium hexafluorophosphate) or COMU ((1-Cyano-2-ethoxy-2-oxoethylidenaminooxy)dimethylamino-morpholino-carbenium hexafluorophosphate).

57. The process of any one of embodiments 50-56 wherein the base is DIPEA (diisopropylethylamime).

58. In certain embodiments a process is provided to prepare a compound of Formula XII by reacting a compound of Formula XI with the dihydroquinine salt of isopropyl (hydroxy(phenoxy)phosphoryl)-L-alaninate with an activator and a base to afford a protected diastereomerically enriched Sₚ-phosphoramidate nucleotide of Formula XII wherein the Sₚ-diastereomer is in excess of the Rₚ-diastereomer:

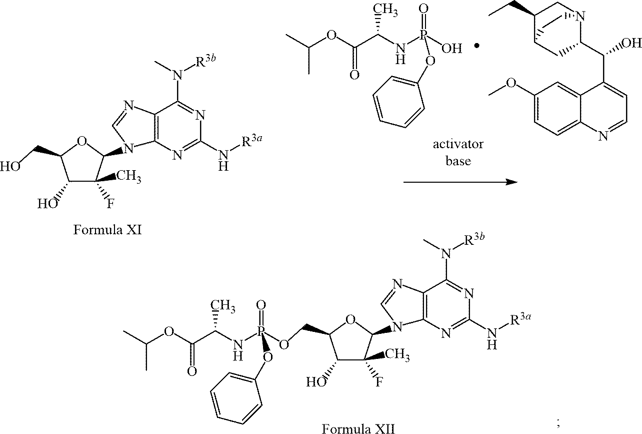

wherein:

R^{3a} and R^{3b} are independently selected from tert-butoxycarbonyl-(Boc), benzyloxycarbonyl-(Cbz), benzyl, and p-methoxybenzyl.

59. The process of embodiment 58 further comprising converting a compound of Formula VIII to the compound of Formula XI:

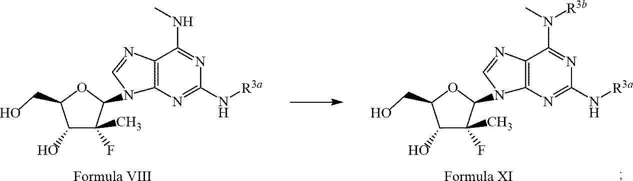

60. The process of embodiment 58 or 59 further comprising converting the compound of Formula XII into Compound 1:

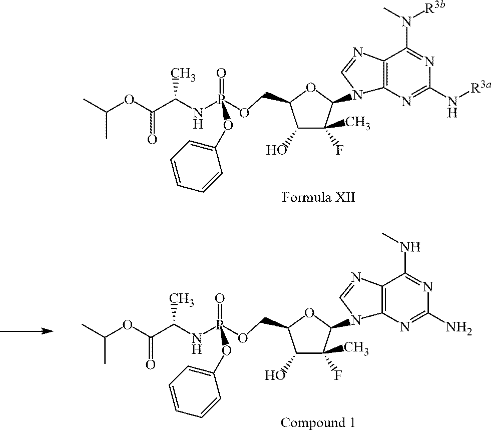

61. The process of embodiment 60 wherein the compound of Formula XII is converted to Compound 1 with HCl in DCM.

62. The process of embodiment 60 wherein the compound of Formula XII is converted to Compound 1 with Pd/C and hydrogen.

63. The process of embodiment 60 wherein the compound of Formula XII is converted to Compound 1 with DBU and MeOH.

64. The process of any one of embodiments 58-63 wherein the activator is HATU (O-(7-azabenzotriazol-1-yl)-1,1,3,3-tetramethyluronium hexafluorophosphate) or COMU ((1-Cyano-2-ethoxy-2-oxoethylidenaminooxy)dimethylamino-morpholino-carbenium hexafluorophosphate).

65. The process of any one of embodiments 58-64 wherein the base is DIPEA (diisopropylethylamine).

66. In certain embodiments a process is provided to prepare a compound of Formula XIV by reacting a compound of Formula XIII with the dihydroquinine salt of isopropyl (hydroxy(phenoxy)phosphoryl)-L-alaninate with an activator and a base to afford a protected diastereomerically enriched Sₚ-phosphoramidate nucleotide of Formula XIV wherein the Sₚ-diastereomer is in excess of the Rₚ-diastereomer:

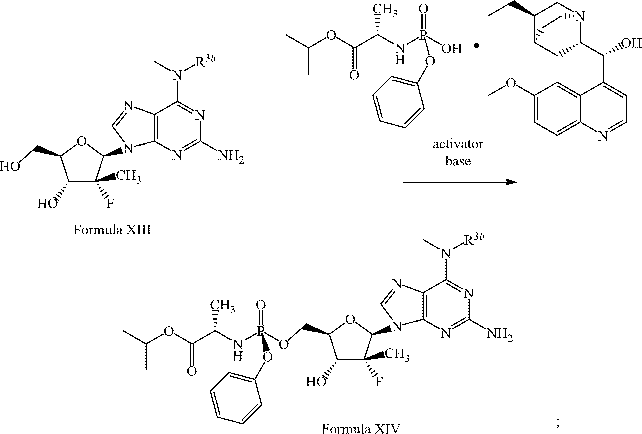

wherein:

R^{3a} and R^{3b} are independently selected from tert-butoxycarbonyl-(Boc), benzyloxycarbonyl-(Cbz), benzyl, and p-methoxybenzyl.

67. The process of embodiment 66 further comprising converting Compound 2 to the compound of Formula XIII:

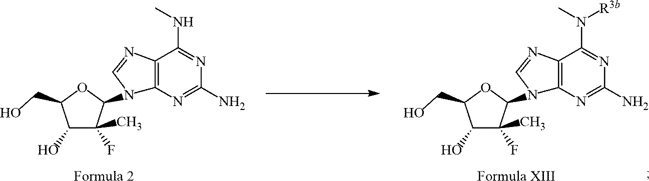

68. The process of embodiment 66 or 67 further comprising converting the compound of Formula XIV into Compound 1:

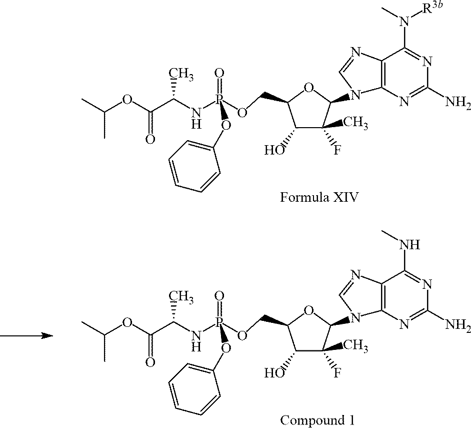

69. The process of embodiment 68 wherein the compound of Formula XIV is converted to Compound 1 with HCl in DCM.

70. The process of embodiment 68 wherein the compound of Formula XIV is converted to Compound 1 with Pd/C and hydrogen.

71. The process of embodiment 68 wherein the compound of Formula XIV is converted to Compound 1 with DBU and MeOH.

72. The process of any one of embodiments 66-71 wherein the activator is HATU (O-(7-azabenzotriazol-1-yl)-1,1,3,3-tetramethyluronium hexafluorophosphate) or COMU ((1-cyano-2-ethoxy-2-oxoethylidenaminooxy)dimethylamino-morpholino-carbenium hexafluorophosphate).

73. The process of any one of embodiments 66-72 wherein the base is DIPEA (diisopropylethylamime).

74. In certain embodiments a process is provided to prepare a compound of Formula XVII comprising the steps:

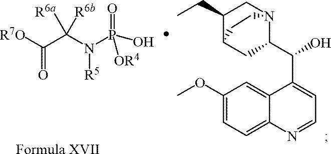

(a) Coupling of an appropriately substituted dichlorophosphate with benzyl alcohol to generate an appropriately substituted benzyl phosphorochloridate without isolation, and subsequently reacting the substituted benzyl phosphorochloridate with the appropriately substituted amino acid to afford a benzylphosphoramidate

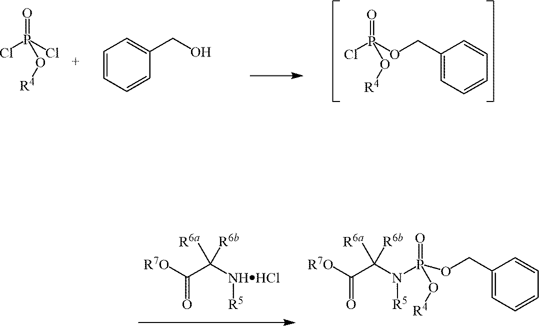

(b) Debenzylating the benzyl phosphoramidate in the presence of a chiral tertiary amine selected from quinine and dihydroquinine to afford the compound of Formula XVII

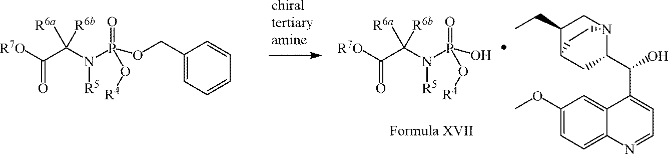

wherein:
   R⁴ is hydrogen, C₁₋₆alkyl, C₃₋₇cycloalkyl, or aryl;
   R⁵ is hydrogen or C₁₋₆alkyl;
   R^{6a} and R^{6b} are independently selected from hydrogen, C₁₋₆alkyl, or C₃₋₇cycloalkyl; and
   R⁷ is hydrogen, C₁₋₆alkyl, C₁₋₆haloalkyl, or C₃₋₇cycloalkyl.

75. The process of embodiment 74 wherein R⁴ is aryl.

76. The process of embodiment 74 or 75 wherein R⁴ is phenyl.

77. The process of any one of embodiments 74-76 wherein R⁵ is hydrogen.

78. The process of any one of embodiments 74-77 wherein at least one of R^{6a} and R^{6b} is hydrogen.

79. The process of any one of embodiments 74-78 wherein R^{6a} and R^{6b} are hydrogen and methyl.

80. The process of any one of embodiments 74-79 wherein R⁷ is C₁₋₆alkyl.

81. The process of any one of embodiments 74-80 wherein R⁷ is isopropyl.

82. The process of any one of embodiments 74-81 wherein step (b) is performed with Pd/C and hydrogen.

83. The process of any one of embodiments 74-82 wherein dihydroquinine is used in step (b).

84. In certain embodiments compound of Formula IIA, IIIA, II′, or III′

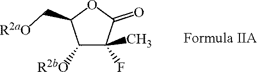

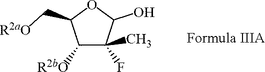

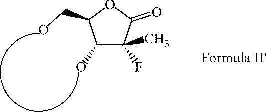

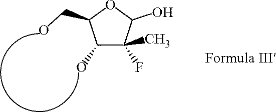

or a pharmaceutically acceptable salt thereof is provided, wherein:
R^{2a} and R^{2b} are oxygen protecting groups;
at least one of R^{2a} and R^{2b} is —C(O)OC₁₋₂₀alkyl, —C(O)OC₂₋₂₀alkenyl, or —C(O)NR^{10a}R^{10b};
R^{10a} and R^{10b} are independently selected from hydrogen, C₁₋₂₀alkyl, C₂₋₂₀alkenyl, aryl, arylalkyl, heteroaryl, and heteroarylalkyl
R^{2a} and R^{2b} can independently be optionally substituted with a substituent selected from alkoxy, hydroxy, nitro, bromo, chloro, fluoro, azido, and haloalkyl; and the bridge structure is selected from:

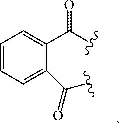

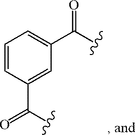

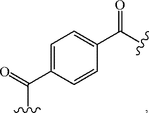

optionally substituted with substituents selected from alkoxy, hydroxy, nitro, bromo, chloro, fluoro, azido, and haloalkyl.

85. The compound of embodiment 84 wherein R^{2a} and R^{2b} are —C(O)OtBu.

86. The compound of embodiment 84 wherein R^{2a} and R^{2b} are —C(O)OMe.

87. In certain embodiments a compound of Formula XVII:

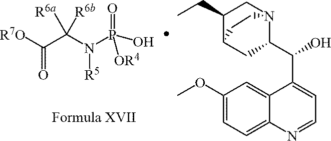

is provided; wherein:
R⁴ is hydrogen, C₁₋₆alkyl, C₃₋₇cycloalkyl, or aryl;
R⁵ is hydrogen or C₁₋₆alkyl;
R^{6a} and R^{6b} are independently selected from hydrogen, C₁₋₆alkyl, or C₃₋₇cycloalkyl; and
R⁷ is hydrogen, C₁₋₆alkyl, C₁₋₆haloalkyl, or C₃₋₇cycloalkyl.

88. The compound of embodiment 87 wherein R⁴ is aryl.

89. The compound of embodiment 87 or 88 wherein R⁴ is phenyl.

90. The compound of any one of embodiments 87-89 wherein R⁵ is hydrogen.

91. The compound of any one of embodiments 87-90 wherein at least one of R^{6a} and R^{6b} is hydrogen.

92. The compound of any one of embodiments 87-91 wherein R^{6a} and R^{6b} are hydrogen and methyl.

93. The compound of any one of embodiments 87-92 wherein R⁷ is C₁₋₆alkyl.

94. The compound of any one of embodiments 87-93 wherein R⁷ is isopropyl.

95. The compound of any one of embodiments 87-94 of structure:

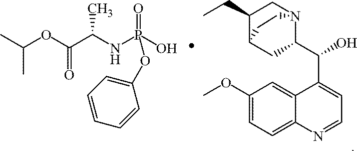

96. In certain embodiments a compound of structure:

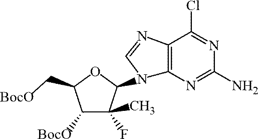

is provided.

97. In certain embodiments a compound of structure:

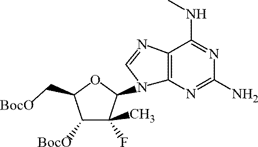

is provided.

### Coupling of Dihydroquinine Salt of Isopropyl (hydroxy(phenoxy)phosphoryl)-L-alaninate with Nucleoside Compound 2

In one aspect of the present invention, the process for synthesizing the diastereomerically pure Sₚ-phosphoramidate nucleotide of Compound 1 comprises the steps of:
(a) contacting nucleoside Compound 2 with the dihydroquinine salt of isopropyl (hydroxy(phenoxy)phosphoryl)-L-alaninate in the presence of a specified activator as described herein and base to afford diastereomerically enriched Sₚ-phosphoramidate nucleotide Compound 1 wherein the Sₚ-diastereomer is in substantial excess over the Rₚ-diastereomer:

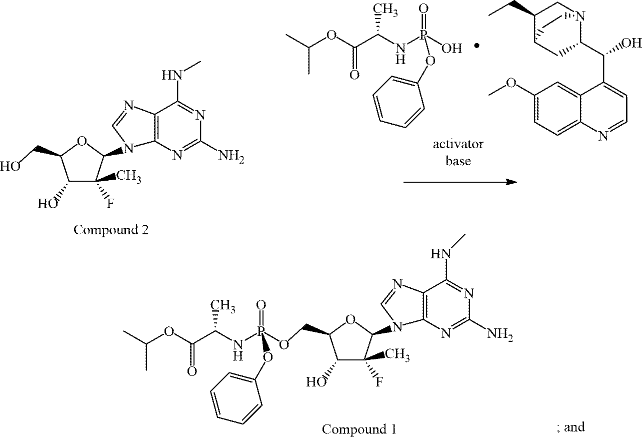

(b) further optionally purifying, e.g., by selective crystallization, the diastereomerically enriched Sₚ-phosphoramidate nucleotide Compound 1 to afford the diastereomerically pure Sₚ-Compound 1 wherein the diastereomerically purity is greater than about 90%, or even greater than about 95% or even about 99% or greater.

In one embodiment, the activator is COMU ((1-Cyano-2-ethoxy-2-oxoethylidenaminooxy)dimethylamino-morpholino-carbenium hexafluorophosphate).

In one embodiment, the activator is HATU (O-(7-azabenzotriazol-1-yl)-1,1,3,3-tetramethyluronium hexafluorophosphate).

In some embodiments, an alternative activator is used, typically a benzotriazole-based activator, including, but not limited to HOBt ((1-hydroxybenzotriazole), PyBOP (benzotriazol-1-yloxytri(pyrrolidino)phosphonium hexafluorophosphate), HBTU (3-[bis(dimethylamino)methyliumyl]-3H-benzotriazol-1-oxide hexafluorophosphate), HCTU (2-(6-chloro-1H-benzotriazole-1-yl)-1,1,3,3-tetramethylaminium hexafluorophosphate), and TBTU (O-benzotriazol-1-yl-1,1,3,3-tetramethyluronium tetrafluoroborate).

Additional non-limiting examples of alternative activators include AOMP (5-(7-azabenzotriazol-1-yloxy)-3,4-dihydro-1-methyl 2H-pyrrolium hexachloroantimonate), AOP ((7-azabenzotriazol-1-yl)oxytris(dimethylamino)phosphonium hexafluorophosphate), BDDC (bis(4-(2,2-dimethyl-1,3-dioxolyl)-methyl-carbodiimide), BDMP (5-(1H-benzotriazol-1-yloxy)-3,4-dihydro-1-methyl 2H-pyrrolium hexachloroantimonate), BDP (benzotriazol-1-yl diethylphosphate), BEC (N-tert-butyl-N′-ethylcarbodiimide), BEMT (2-bromo-3-ethyl-4-methylthiazolium tetrafluoroborate), BEP (2-bromo-1-ethyl pyridinium tetrafluoroborate), BEPH (2-bromo-1-ethyl pyridinium hexachloroantimonate), BMP-Cl (N,N′-bismorpholinophosphinic chloride), Boc (t-butyloxycarbonyl), BOMP (2-(benzotriazol-1-yloxy)-1,3-dimethyl-2-pyrrollidin-1-yl-1,3,2- diazaphospholidinium hexafluorophosphate), BOP (benzotriazol-1-yloxytris(dimethylamino)phosphonium hexafluorophosphate), BOP-Cl (N,N′-bis(2-oxo-3-oxazolidinyl)phosphinic chloride), BroP (bromotris(dimethylamino)phosphonium hexafluorophosphate), Bsmoc (1,1-dioxobenzobthiophene-2-ylmethyloxycarbonyl), Bspoc (2-(tert-butylsulfonyl)-2-propyloxycarbonyl), Bts-Fmoc (2,7-bis(trimethylsilyl)-9-fluorenylmethyloxycarbonyl), BTFFH (bis(tetramethylene) fluoroformamidinium hexafluorophosphate), BPMP (1-(1H-benzotriazol-1-yloxy)phenylmethylene pyrrollidinium hexachloroantimonate), BTC (triphosgene), BTCFH (bis(tetramethylene)chlororformamidinium hexafluorophosphate, (PyClU)), Bts-Cl (benzothiazol-2-sulfonyl chloride), Cbz, Z (benzyloxycarbonyl), CDMT (2-chloro-4,6-dimethoxy-1,3,5-triazine), CC (cyanuric chloride), CDPOP (pentachlorophenyl diphenylphosphate), CDPP (pentachlorophenyl diphenylphosphinate), CF (cyanuric fluoride), CF₃-BOP ([6-(trifluoromethyl)benzotriazol-1-yl)-Noxy tris(dimethylamino)phosphonium hexafluorophosphate), CF₃-HBTU (2-6-(trifluoromethyl)-benzotriazol-1-yl)-1,1,3,3-tetramethyluronium hexafluorophosphate), CF₃-NO₂⁻ PyBOP (4-nitro-6-(trifluoromethyl)benzotriazol-1-yl)-oxytris(pyrrollidino)), 6-Cl-HOBI (6-chloro-N-hydroxy-2-phenylbenzimidazole phosphonium hexafluorophosphate), CF₃-PyBOP (6-(trifluoromethyl)-benzotriazol-1-yl)-N-oxytris (pyrrollidino)phosphonium hexafluorophosphate), 6-Cl-HOBt (6-chloro-1-hydroxybenzotriazole), CIC (N-cyclohexyl,N′-isopropyl carbodiimide), CIP (2-chloro-1,3-dimethylimidazolidinium hexafluorophosphate), CloP (chloro-tris(dimethylamino)phosphonium hexafluorophosphate), CMBI (2-chloro-1,3-dimethyl 1H-benzimidazolium hexafluorophosphate), CMPI (2-chloro-1-methylpyridinium iodide), COMU (1-(1-(cyano-2-ethoxy-2-oxoethylideneaminooxy)-dimethylaminomorpholinomethylene)methanaminium hexafluorophosphate), Cpt-Cl (1-oxo-chlorophospholane), CPC (N,N′-dicyclopentylcarbodiimide), CPP (2-chloro-1,3-dimethylpyrimidiniumhexafluorophosphate), DCC (N,N′-dicyclohexylcarbodiimide), DCMT (2,4-dichloro-6-methoxy-1,3,5-triazine), DECP (diethylcyanophosphonate), DEPAT (3-(diethoxyphosphoryloxy)-1,2,3-pyridino-btriazin-4-(3H)-one), DKP (2,5-Diketopiperazine), DEBP (diethyl 2-(3-oxo-2,3-dihydro-1,2-benzisosulfonazolyl)phosphonate), DEPB (diethyl phosphorobromidate), DEPBO (N-diethoxyphosphoryl benzoxazolone), DEPBT (3-( diethoxyphosphoryl oxy)-1,2,3-benzotriazin-4(3 H)-one), DEPC (diphenyl phosphorochloridate), DEFFH (1,2-diethyl-3,3-tetramethylne fluoroformamidinium hexafluorophosphate), DFIH (1,3-dimethyl-2-fluoro-4,5-dihydro-1H-imidazolium hexafluorophosphate), DIC (N,N′-diisopropylcarbodiimide), DMCH (N-(chloro(morpholino)methylene)-N-methylmethanaminium hexafluorophosphate), DMCT (2-chloro-4,6-dimethyl-1,3,5-triazine), DMFFH (1,2-dimethyl-3,3-tetramethylenefluoroformamidinium hexafluorophosphate), DMFH (N-(fluoro(morpholino)methylene)-N-methylmethanaminium hexafluorophosphate), DMTMM (4-(4,6-dimethoxy[1,3,5]triazin-2-y1)-4-methylmorpholinium chloride), DNAs (3H-[1,2,3]triazolo [4,5-b]pyridin-3-yl 2,4-dinitrobenzenesulfonate), DNBs (1H-benzo [ d] [1,2,3]triazol-1-yl 2,4-dinitrobenzenesulfonate), DOMP (5-(30,40-dihydro-40-oxo-10,20,30-benzotriazin-3 0-y loxy)-3,4-dihydro-1- methyl-2H -pyrrolium hexachloroantimonate), DOPBO (N-(2-oxo-1,3,2-dioxaphosphorinany 1 )-benzoxazo lone), DOPBT (3-[0-(2-oxo-1,3,2-dioxaphosphorinanyl)-oxy ]-1,2,3-benzotriazin-4(3H)-one), DOEPBI (phosphoric acid diethyl ester 2-phenylbenzimidazol-1-yl ester), DOPPBI (phosphoric acid diphenyl-2-phenylbenzimidazol-1-yl ester), DPPBI (diphenylphosphinic acid 2-phenylbenzimidazol-1-yl ester), DPPAT (3-(diphenoxyphosphoryloxy)-1,2,3-pyridino-btriazin-4-(3H)-one), DPP-Cl (diphenylphosphinic chloride), DPPA (diphenylphosphoryl azide), Dtb-Fmoc (2,7-di-tert-butyl-9-fluorenylmethyloxycarbonyl), EDC (1-[3-(dimethylamino )propyl]-3-ethylcarbodiimide hydrochloride), FDMP (3,5-bis(trifluoromethylphenyl)phenyl diphenylphosphinate), FDPP (pentafluorophenyl diphenyl phosphinate), FEP (2-fluoro-1-ethyl pyridinium tetrafluoroborate), FEPH (2-fluoro-1-ethyl pyridinium hexachloroantimonate), FIP (2-fluoro-1,3-dimethylimidazolidiniumhexafluorophosphate), Fmoc (9-fluorenylmethyloxcarbonyl), FOMP 2H-pyrrolium(5-(pentafluorophenyloxy)-3,4-dihydro-1-methyl-hexachloroantimonate), HAE2PipU (O-(1 H-1,2,3-triazolo[4,5-b ]pyridin-1-yl)-1,1-diethyl-3,3-pentarnethyleneuronium), HAE2PyU (O-(1H-1,2,3-triazolo[4,5-b]pyridin-1-yl)-1,1-diethyl-3,3-tetramethyleneuronium hexafluorophosphate), HAM2PipU (O-(1 H-1,2,3-triazolo[4,5-b ]pyridin-1-yl)-1,1-dimethyl-3,3-pentamethyleneuronium hexafluorophosphate), HAM2PyU (O-(1H-1,2,3-triazolo[4,5-b]pyridin-1-yl)-1,1-dimethyl-3,3-tetramethyleneuronium hexafluorophosphate), HAMTU (O-(7-azabenzotriazol-1-yl)-1,3-dimethyl-1,3-trimethyleneuronium hexafluorophosphate), HAMDU (O-(7-azabenzotriazol-1-yl)-1,3-dimethyl-1,3-dimethyleneuronium hexafluorophosphate), HAPipU (O-(7-azabenzotriazol-1-yl)-1,1,3,3-bis(pentamethylene)uronium hexafluorophosphate), HAPyU (1-(1-pyrrolidinyl-1H-1,2,3-triazolo[4,5-b]-pyridin-1-ylmethylene)pyrrolidinium hexafluorophosphate N-oxide), HAPyTU (1-(1-pyrrolidinyl-1 H-1,2,3-triazolo[4,5-b]-pyridin-1-ylmethylene)pyrrolidinium hexafluorophosphate N-sulfide), HAPTU (7-azabenzotriazol-1-yl)-1,1,3trimethyl-1-phenyluronium hexafluorophosphate), HATTU (S-(7-azabenzotriazol-1-yl)-1,1,3,3-tetramethyluronium hexafluorophosphate), HATU (O-(1 H-1,2,3-triazolo[4,5-b]pyridin-1-yl)-1,1,3,3-tetraethyluronium hexafluorophosphate), HBE2PipU (O-(1 H-benzotriazol-1-yl)-1,1-diethyl-3,3-pentamethyleneuronium hexafluorophosphate), HBE2PyU (O-(1 H-benzotriazol-1-yl)-1,1-diethyl-3,3-tetramethyleneuronium hexafluorophosphate), HBM2PipU (O-(1H-benzotriazol-1-yl)-1,1-dimethyl-3,3-pentamethyleneuronium hexafluorophosphate), HBM2PyU (O-(1H-benzotriazol-1-yl)-1,1-dimethyl-3,3-tetramethyleneuronium hexafluorophosphate), HBMTU (O-(benzotriazol-1-yl)-1,3-dimethyl-1,3-trimethyleneuronium hexafluorophosphate), HBPTU ((7-benzotriazol-yl)-1,1,3-trimethyl-1-phenyluronium hexafluorophosphate), HBTeU (O-(1 H-benzotriazol-1-yl)-1,1,3,3-tetraethyluronium hexafluorophosphate), HBMDU (O-(benzotriazol-1-yl)-1,3-dimethyl-1,3-dimethyleneuronium hexafluorophosphate), HBPipU (O-(benzotriazol-1-yl)-1,1,3,3-bis(pentamethylene)uronium hexafluorophosphate), HBPyU (O-(benzotriazol-1-yl)oxybis(pyrrolidino)-uronium hexafluorophosphate), HDATU (O-(3,4-dihydro-4-oxo-5-azabenzo-1,2,3-triazin-3-yl)-1,1,3,3-tetramethyluronium hexafluorophosphate), HDAPyU (O-(3,4-dihydro-4-oxo-5-azabenzo-1,2,3-triazin-3-yl)-1,1,3,3-bis(tetramethylene)uronium hexafluorophosphate), HDTU (O-(3,4-dihydro-4-oxo-1,2,3-benzotriazin-3-yl)-1,1,3,3-tetramethyluronium hexafluorophosphate), HDATU (O-(3,4-dihydro-4-oxo-5-azabenzo-\1,2,3-triazin-3-yl)-\1,1,3,3-tetramethyluronium hexafluorophosphate), HDMA (1-((dimethylamino)-(morpholino)methylene)-1H-[1,2,3]triazolo[4,5-b]pyridinium hexafluorophosphate-3-oxide), 4-HDMA (3-((dimethylamino)-(morpholino)methylene)-1H-[1,2,3]triazolo[4,5-b]pyridinium hexafluorophosphate-1-oxide), HDMB (1-((dimethylamino)(morpholino)methylene)-lH-benzotriazolium hexafluorophosphate-3-oxide), HDMC (6-chloro-1-((dimethylamino)(morpholino)-methylene)-1H-benzotriazolium hexafluorophosphate-3-oxide), 6-6-HDMFB (6-trifluoromethyl-1-((dimethylamino)(morpholino)methylene)-lH-benzotriazolium hexafluorophosphate-3-oxide), HDMODC (1-[(1-(dicyanomethyleneaminooxy)-dimethylaminomorpholinomethylene)]methanaminium hexafluorophosphate), HDMODeC (1-[(1,3-diethyoxy-1,3-dioxopropan-2-ylideneaminooxy)-dimethylamino-morpholinomethylene)]methanaminium hexafluorophosphate), HDMOPC (N-[(cyano(pyridine-2-yl)methyleneaminooxy)-(dimethylamino)methylene ]\-N-\morpholinomethanaminium hexafluorophosphate), HDMP (1-((dimethylamino)(morpholino))oxypyrrolidine-2,5-dione methanaminium hexafluorophosphate), HDMPfp (1-((dimethylamino)-(morpholino))oxypentafluorophenylmetheniminium hexafluorophosphate), HDmPyODC (1-[(1-(cyano-2-ethoxy-2-oxoethylideneaminooxy)-dimethylaminopyrrolodinomethylene)]methanaminium hexafluorophosphate), HDPyU (O-(3,4-dihydro-4-oxo-1,2,3-benzotriazin-3-yl)-1,1,3,3-bis(tetramethylene)uronium hexafluorophosphate), HDTMA (1-((dimethylamino)(thiomorpholino)methylene)-1H-[1,2,3]triazolo[4,5-b]pyridinium hexafluorophosphate-3-oxide), HDTMB (1-((dimethylamino )(thiomorpholino )methylene)-1H-benzotriazolium hexafluorophosphate-3-oxide), HDmPyODeC (1-[(1,3-diethyoxy-1,3-dioxopropan-2-ylideneaminooxy)-dimethylamino pyrrolodinomethylene)]methanaminium hexafluorophosphate), HDmPyOC (1-[(1-(cyano-2-ethoxy-2-oxoethylideneaminooxy)-dimethylamino-pyrrolodinomethylene)]methanaminium hexafluorophosphate), HMPyODC (1-((dicyanomethyleneaminooxy)morpholinomethylene)pyrrolidinium hexafluorophosphoate), HMPA (hexamethylphosphoramide), HMPyOC (1-((1-cyano-2-ethoxy-2-oxoethylideneaminooxy) (morpholino)methylene)pyrrolidinium hexafluorophosphate), HOAt (1-hydroxy-7-azabenzotriazole), 4-HOAt (4-aza-1-hydroxybenzotriazole), 5-HOAt (5-aza-1-hydroxybenzotriazole), 6-HOAt (6-aza-1-hydroxybenzotriazole), HOBI (N-hydroxy-2-phenylbenzimidazole), HOCt (ethyl-1-hydroxy-1H-\1,2,3-triazole-4-carboxylate), HODhbt (3,4-dihydro-3-hydroxy-4-oxo-\1,2,3-benzotriazine), HODhad (3-hydroxy-4-oxo-3,4-dihydro-5-azabenzo-1,3-diazene), HODhat (3-hydroxy-4-oxo-3,4-dihydro-5-azabenzo-\1,2,3-triazene), HODT (S-(1-oxido-2-pyridinyl)-1,3-dimethyl-1,3-trimethylenethiouronium hexafluorophosphate), HOSu (N-hydroxysuccinimide), HOI (N-hydroxyindolin-2-one), 6-NO₂-HOBt (1-hydroxy-6-nitrobenzotriazole), HONP (p-nitrophenyl active ester), HOPy (1-hydroxy-2-pyridinone), 6-CF₃-HOBt (6-trifluoromethyl-1-hydroxy benzotriazole), PS-SO₂-HOBt (polymer-supported 1-hydroxy-6-disulfoxide benzotriazole), PS-HOSu (polymer-supported N-hydroxysuccinimide), PS-DCT (polymer-supported 2,4-dichloro-\1,3,5-triazine), HONB (N-hydroxy-5-norbornene-endo-2,3-dicarboxyimide), HOTT (S-(1-oxido-2-pyridinyl)-\1,1,3,3-tetramethylthiouronium hexafluorophosphate), HOTU (O-[cyano(ethoxycarbonyl)methyleneamino]-N,N,N′,N′-tetramethyluronium hexafluorophosphate), HPyOPfp (N,N,N′,N′-bis (tetramethylene)-O-pentafluorophenyluronium hexafluorophosphate), HPFTU (N,N,N′,N′-bis(tetramethylene)-O-pentafluorophenyluronium hexafluorophosphate), HPTU (2-(2-oxo-1(2H)-pyridyl-1,1,3,3-tetramethyluronium hexafluorophosphate), HPyONP (N,N,N′,N′-bis(tetramethylene)-O-2-nitrophenyluronium hexafluorophosphate), HPyOTCp (N,N,N′,N′-bis(tetramethylene)-O-pentafluorophenyluronium hexafluorophosphate), HPySPfp (N,N,N′,N′-bis(tetramethylene)-S-pentafluorothiophenyluronium hexafluorophosphate), HSTU (2-succinimido-1,1,3,3-tetramethyluronium hexafluorophosphate), HTODC (O-[(dicyanomethylidene)-amino]-1,1,3,3-tetramethyluronium hexafluorophosphate), HTODeC (O-[(diethoxycarbonylmethylidene)amino]-\1,1,3,3-tetramethyluronium hexafluorophosphate), HTOPC (N-[(cyano(pyridine-2-yl)methyleneaminooxy)-(dimethylamino)methylene)-N-methylmethanaminium hexafluorophosphate), MPTA (methylmethanarninium tetrafluo), MPTO (3-dimethylphosphinothioyl-2(3H)-oxazolone), Mspoc (2-methylsulfonyl-3-phenyl-1-prop-2-enyloxycarbonyl), Mukaiyama’s reagent (2-chloro-1-methylpyridinium iodide), NAs (3-((naphthalen-2-ylsulfonyl)methyl)-3H-[1,2,3]-triazolo[4,5-b]pyridine), 2-NAs (3H-[1,2,3]triazolo[4,5-b]pyridin-3-yl 2-nitrobenzenesulfonate), 4-NAs (3H-[1,2,3 ]triazolo[4,5-b]pyridin-3-yl 4-nitrobenzenesulfonate), NBs (1-((naphthalen-2-ylsulfonyl)methyl)-1H-benzo-[d] [1,2,3]triazole), 2-NBs (lH-benzo[d][1,2,3]triazol-1-yl 2-nitrobenzenesulfonate), 4-NBs (1H-benzo[d] [1,2,3]triazol-1-yl 4-nitrobenzenesulfonate), NDPP (norborn-5-ene-2,3-dicarboximidodiphenylphosphate), N-HATU (N-[(dimethylamino)-1 H-1,2,3-triazolo[4,5-b]-pyridin-1-ylmethylene]-N-methylmethanaminium hexafluorophosphate N-oxide), N-CF₃-HBTU (N-[6-trifluoromethyl(1H-benzotriazol-1-yl)-(dimethylamino)methylene]-N-methylmethanaminium hexafluorophosphate N-oxide), N-CF₃-TBTU (N-[6-trifluoromethyl(1H-benzotriazol-1-yl)-(dimethylamino)methylene]-N-methylmethanaminium tetrafluoroborate N-oxide), N-HAPyU (1-(1-pyrrolidinyl-1H-1,2,3-triazolo[4,5-b]pyridin-1-ylmethylene)pyrrolidinium hexafluorophosphate N-oxide), N-HATTU (N-[(dimethylamino)-1 H-1,2,3-triazolo[4,5-b]-pyridin-1-ylmethylene]-N-methylmethanaminium hexafluorophosphate N-sulfide), N-HBPyU ((1H-benzotriazol-1-yl)(1-pyrrolidinylmethylene)pyrrolidinium hexafluorophosphate N-oxide), N-HBTU (N-[(1H-benzotriazol-1-yl)(dimethylamino)-methylene]-N-methylmethanaminium hexafluorophosphate N-oxide), N-TATU (N-[(dimethylamino)-1H-1,2,3-triazolo[4,5-b]-pyridin-1-ylmethylene]-N-methylmethananinium tetrafluoroborate N-oxide), N-TBTU (N-[(1H-benzotriazol-1-yl)(dimethylamino)-methylene]-N-methylmethanarninium tetrafluoroborate N-oxide), NDPP (norborn-5-ene-2,3-dicarboximidodiphenylphosphate), NMM (N-methylmorpholine), NO₂-PyBOP (6-nitrobenzotriazol-1-yloxy)tris(pyrrolidino)phosphonium hexafluorophosphate), Oxyma (ethyl 2-cyano-2-(hydroxyimino)acetate), PIC (N-phenyl,N-isopropylcarbod-imide), PS (polymer supported), PS-DCC (polymer cyclohexylcarboniimide), PS-EDC (polymer 1-ethyl-3-(30-dimethylaminopropyl)-carbodiimide), PEC (N-ethyl, N-phenylcarbodiimide), PS-TBTU (N-[(1H-benzotriazol-1-yl)(dimethylamino)-methylene]-N-Xmethylmethanarninium tetrafluoroborate N-oxide), PTF (benzyltriphenylphosphonium dihydrogen trifluoride), PyAOP ((7-azabenzotriazol-1-yl)oxy]tris(pyrrolidino)phosphonium hexafluorophosphate), PyBroP (bromotri(pyrrolidino)phosphonium hexafluorophosphate), PyCloP (chlorotri(pyrrolidino)phosphoniumhexafluorophosphate), PyDOP ([(3,4-dihydro-4-oxo-1,2,3-benzotriazin-3-yl)oxy]-tris(pyrrolidino)phosphonium hexafluorophosphate), PyCloK (( 6-chloro-benzotriazol-1-yloxy)tris(pyrrolidino)phosphonium hexafluorophosphate), PyPOP ((pentafluoropheny 1oxy )tris(pyrrolidino)phosphonium hexafluorophosphate), PyDAOP ([(3,4-dihydro-4-oxo-5-azabenzo-1,2,3-triazin-3-yl]tris(pyrrolidino)phosphonium hexafluorophosphate), PyFOP ([[6-(trifluoromethyl)benzotriazol-1-yl]oxy]-tris(pyrrolidino)phosphonium hexafluorophosphate), PyFNBOP ([4-nitro-6-(trifluoromethyl)benzotriazol-1-yl)-oxy]tris(pyrrolidino)phosphonium hexafluorophosphate), PyNOP ([(6-nitrobenzotriazol-1-yl)oxy]tris(pyrrolidino)phosphonium hexafluorophosphate), PyOxm (O-[(cyano(ethoxycarbonyl)methyliden)-amino]-yloxytri(pyrrolidino)phosphonium hexafluorophosphate), PyTOP ((pyridyl-2-thio)tris(pyrrolidino)phosphonium hexafluorophosphate), SOMP (5-(succinimidyloxy)-3,4-dihydro-1-methyl 2H-pyrrolium hexachloroantimonate), TATU (O-(7-azabenzotriazol-1-yl)-1,1,3,3-tetramethyluronium tetrafluoroborate), TAs (3H-[1,2,3]triazolo [4,5-b]pyridin-3-yl-4-methylbenzenesulfonate), TBs (1H-benzo[d][1,2,3]triazol-1-yl-4-methylbenzenesulfonate), TBCR1 (4-(4,6-dimethoxy-1,3,5-triazin-2-yl)-4-methylmorpholinium tetrafluoroborate), TBCR2 (1-(4,6-dimethoxy-1,3,5-triazin-2-yl)-1-methylpiperydinium tetrafluoroborate), TBCR3 (1-(4,6-dimethoxy-1,3,5-triazin-2-yl)quinuclidinium tetrafluoroborate), TDBTU (2-(3,4-dihydro-4-oxo-1,2,3-benzotriazin-3-yl)-1,1,3,3-tetrarnethyluroniwn tetrafluoroborate), TCFH (tetramethylchloroformamidinium hexafluorophosphate), TCP (2,4,5-trichlorophenyl active ester), TDATU (O-(3,4-dihydro-4-oxo-5-azabenzo-1,2,3-triazin-3-yl)-1,1,3,3-tetrarnethyluronium tetrafluoroborate), TDTU (2-(3,4-dihydro-4-oxo-1,2,3-benzotriazin-3-yl)-1,1,3,3-tetrarnethyluronium tetrafluoroborate), TEFFH (tetraethylfluoroformamidinium hexafluorophosphate), TFMS-DEP (diphenyl(trifluoromethylsulfonyl)phosphoramidate), TFFH (tetramethylfluoroformamidinium hexafluorophosphate), TNTU (2-(5-norbornene-2,3-dicarboximido)-1,1,3,3-tetramethyluronium tetrafluoroborate), TODT (S-(1-oxido-2-pyridinyl)-1,3-dimethyl-1,3-trimethylenethiouronium tetrafluoroborate), TOTT (S-(1-oxido-2-pyridinyl)-1,1,3,3-tetramethylthiouronium tetrafluoroborate), TOTU (O-[cyano(ethoxycarbonyl)methyleneamino]-N,N,N′, N′-tetramethyluronium tetrafluoroborate), TPTU (2-(2-oxo-1 (2H)-pyridyl-1,1,3,3-tetramethyluronium tetrafluoroborate), TSTU (2-succinimido-1,1,3,3-tetrarnethyluroniwntetrafluoro borate), TOPPipU (2[2-0xo-1(2H)-pyridyl]-1,1,3,3-bis(pentamethylene)uronium tetrafluoroborate), T3P (2-propanephosphonic acid anhydride, PPAA), TPFTU (N,N,N′,N′-bis(tetramethylene)-O-pentafluorophenyluronium tetrafluoroborate), TPhTU (2-phthalimido-1,1,3,3-tetramethyluronium tetrafluoroborate), and TPP (triphenylphosphine carbon tetrachloride).

In one embodiment, the base is selected from NR₃ wherein R can be selected independently in each instance from H, alkyl, aryl, heteroaryl, alkenyl, alkynyl, benzyl and allyl, and which typically has at least one, and often two or more, non-hydrogen R groups. In one embodiment, the base is DIPEA (N,N-diisopropylethylamine). In one embodiment, the base is NEt₃ (triethylamine). In an alternative embodiment, the base is selected from DMAP, (S)-C₅Ph₅-DMAP, (R)-C₅Me₅-DMAP, quinidine, quinine, TEA, DBU, TMEDA, imidazole, and K₂CO₃. In one embodiment, the base is quinine. In one embodiment, the base is dihydroquinine.

In one embodiment, the base is a heterocyclic base, including, but not limited to, DABCO, 1,5 diazobicyclo[4.3.0]non-5-ene, 1,8-diazabicyclo[5.4.0]undec-7-ene, DMAP, 2,6 lutidine, piperidine, pyrrole, 3-pyrroline, 2H-pyroole 2-pyrroline, pyrrolidine, carbazole, azaindole, isoindole, indole, 3-H indole, indolizine, indoline, pyridine, piperidine, quinuclidine 4-H quinolizine, isoquinoline, quinoline, 1,8 naphthyridine, tetrahydroquinoline, acridine, oxazole, isoxazole, benoxazole, benzothiazole, isothiazole, thiazole, benzimidazole, imidazole 2, imidazole, imidazolidine, tetrazole, 1,3,4-thiadiazole, 1,2,3-tetrazole, 1,2,4-triazole, benzotriazole, imidazolepyridines, indazole, oxadiazole, phenodiazene, thiomorpholine, dithiane, phenoxazine, morpholine, pyrazole, 2-pyrazoline, pyrazolidine, quinazoline, cinnoline, pyrimidine, pteridine, phthalazine, 1,2,4-triazline, 1,3,5-triazine, piperazine, quinoxaline, phenazine, 1H-indazole, pyridazine, hydantoins, cinnolines, cyclazines, triazolepyridines, 2,2,6,6-tetramethylpiperidine, 2,8,9-triisobutyl-2,5,8,9-tetraaza-1-phosphabicyclo[3.3.3]undecane, 2,8,9-triisopropyl-2,5,8,9-tetraaza-1-phosphabicyclo[3,3,3]undecane, 2,8,9-trimethyl-2,5,8,9-tetraaza-1-phosphabicyclo[3.3.3]undecane and substituted derivatives thereof.

In an alternative embodiment, the base is selected from DMAP, (S)-C₅Ph₅-DMAP, (R)-C₅Me₅-DMAP, quinidine, quinine, TEA, DBU, TMEDA, imidazole, and K₂CO₃. In one embodiment, the base is quinine.

In some embodiments the specified activator is a uronium-type activator selected from HBTU, HATU, COMU, and TFFH and the base is DIPEA. In one embodiment, the activator is COMU and the base is NEt₃. In another embodiment the activator is COMU and the base is DIPEA.

In some embodiments, the specified activator is a benzotriazole-based activator selected from HOBt, PyBOP, HATU, HBTU, HCTU, and TBTU and the base is DIPEA. In one embodiment, the activator is a benzotriazole-based activator selected from HOBt, PyBOP, HATU, HBTU, HCTU, and TBTU and the base is NEt₃. In one embodiment, the activator is HATU and the base is DIPEA. In one embodiment, the activator is HATU and the base is NEt₃.

In an alternative embodiment, the quinine salt of isopropyl (hydroxy(phenoxy)phosphoryl)-L-alaninate is coupled to Compound 2 in step (a):

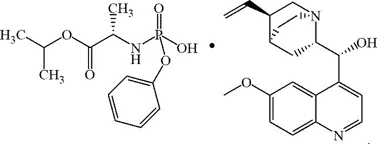

In one aspect of the present invention, the manufacture of the dihydroquinine salt of isopropyl (hydroxy(phenoxy)phosphoryl)-L-alaninate comprises the steps of:
(1.1.a) the coupling of phenyl dichlorophosphate with benzyl alcohol to generate the benzyl phenyl phosphorochloridate in situ that is subsequently reacted with L-alanine isopropyl ester hydrochloride to afford isopropyl ((benzyloxy)(phenoxy)phosphoryl)-L-alaninate:

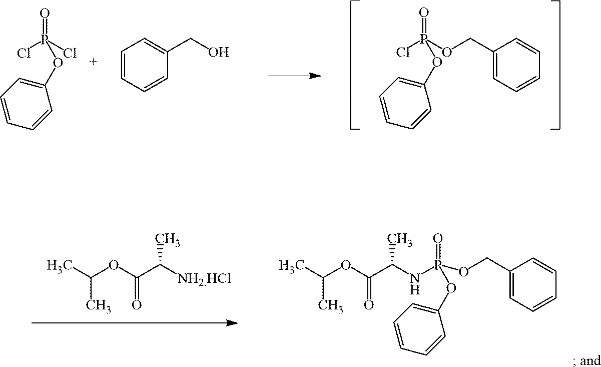

(1.1.b) debenzylation of isopropyl ((benzyloxy)(phenoxy)phosphoryl)-L-alaninate and in situ reduction of quinine to afford the dihydroquinine salt of isopropyl (hydroxy(phenoxy)phosphoryl)-L-alaninate:

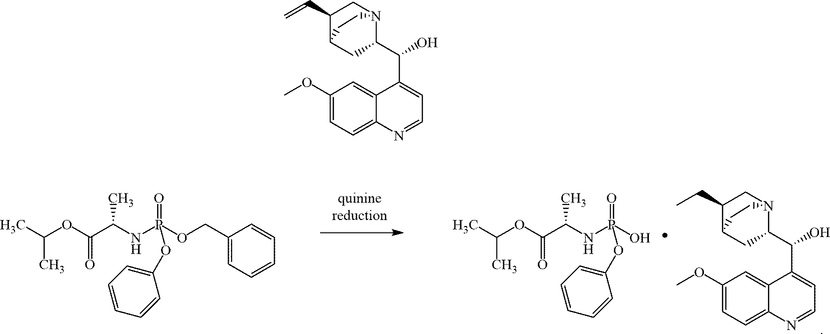

In an alternative embodiment, isopropyl ((benzyloxy)(phenoxy)phosphoryl)-L-alaninate is debenzylated in step (1.b) in the presence of a tertiary amine other than quinine to afford a tertiary amine salt of isopropyl (hydroxy(phenoxy)phosphoryl)-L-alaninate. Non-limiting examples of tertiary amine salts of isopropyl (hydroxy(phenoxy)phosphoryl)-L-alaninate include DBU, DABCO, and diisopropylethylamine:

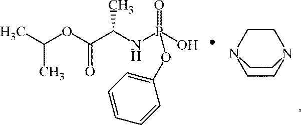

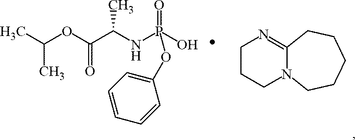

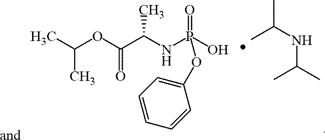

In an alternative aspect of the present invention, the manufacture of the dihydroquinine salt of isopropyl (hydroxy(phenoxy)phosphoryl)-L-alaninate comprises the steps of:
(1.1.a) the coupling of phenyl dichlorophosphate with L-alanine isopropyl ester hydrochloride to afford isopropyl (chloro(phenoxy)phosphoryl)-L-alaninate:

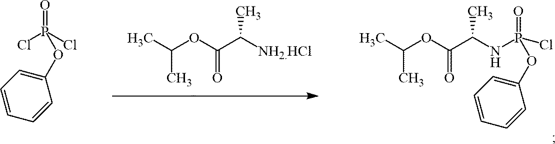

(1.1.b.1) the formation of the DABCO phosphate salt and subsequent treatment with aqueous calcium chloride to afford calcium diphosphoramidate dihydrate salt:

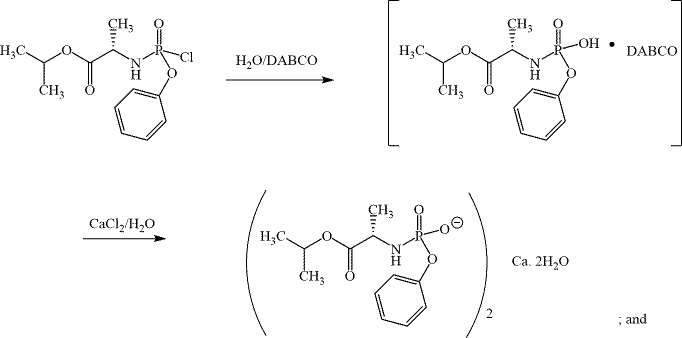

(1.1.c) treatment with dihydroquinine under acidic conditions to afford the dihydroquinine salt of isopropyl (hydroxy(phenoxy)phosphoryl)-L-alaninate:

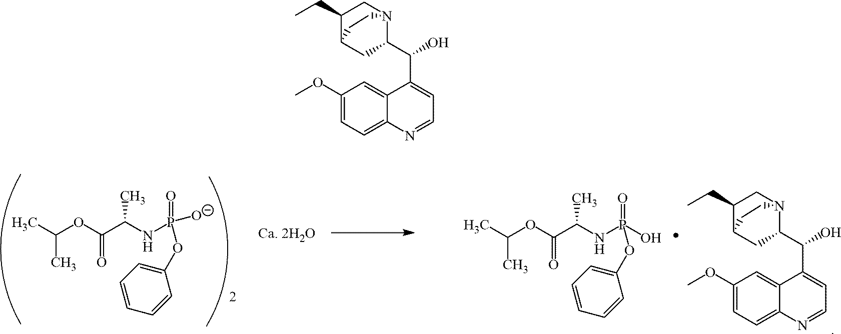

In one embodiment, the calcium diphosphoramidate dihydrate salt is crystalline. In one embodiment, the phosphate salt in step (1.1.b.1) is an alternative tertiary base, including, but not limited to DBU, DMAP, and diisopropylethylamine. In one embodiment, step (1.1.c) is conducted in the presence of HCl.

Additional examples of tertiary amines that can be used in step (1.1.b) or step (1.1.b.1) include 1,5 diazobicyclo[4.3.0]non-5-ene, 1,8-diazabicyclo[5.4.0]undec-7-ene, DMAP (4-dimethylaminopyridine), 2,6 lutidine, piperidine, pyrrole, 3-pyrroline, 2H-pyroole 2-pyrroline, pyrrolidine, carbazole, azaindole, isoindole, indole, 3-H indole, indolizine, indoline, pyridine, piperidine, quinuclidine 4-H quinolizine, isoquinoline, quinoline, 1,8 naphthyridine, tetrahydroquinoline, acridine, oxazole, isoxazole, benoxazole, benzothiazole, isothiazole, thiazole, benzimidazole, imidazole 2, imidazole, imidazolidine, tetrazole, 1,3,4-thiadiazole, 1,2,3-tetrazole, 1,2,4-triazole, benzotriazole, imidazolepyridines, indazole, oxadiazole, phenodiazene, thiomorpholine, dithiane, phenoxazine, morpholine, pyrazole, 2-pyrazoline, pyrazolidine, quinazoline, cinnoline, pyrimidine, pteridine, phthalazine, 1,2,4-triazline, 1,3,5-triazine, piperazine, quinoxaline, phenazine, 1H-indazole, pyridazine, hydantoins, cinnolines, cyclazines, triazolepyridines, 2,2,6,6-tetramethylpiperidine, 2,8,9-triisobutyl-2,5,8,9-tetraaza-1-phosphabicyclo[3.3.3]undecane, 2,8,9-triisopropyl-2,5,8,9-tetraaza-1-phosphabicyclo[3,3,3]undecane, 2,8,9-trimethyl-2,5,8,9-tetraaza-1-phosphabicyclo[3.3.3]undecane and substituted derivatives thereof.

In one embodiment, the tertiary amine is chiral. Non-limiting examples of chiral tertiary amines that can be used in step (1.1.b) or step (1.1.b.1) include tetramisole, quinine, quinine acetate, quinidine gluconate, 9-epi-quinine, 3-hydroxy quinine, quinine N-oxide, hydroquinine 4-chlorobenzoate, hydroquinine-9-phenanthryl ether, quinidine, quinidine N-oxide, hydroquinidine, hydroquinidine 9-phenanthryl ether hydroquinidine 4-methyl-2-quinolyl ether, hydroquinine 4-methyl-2-quinolyl ether, O-desmethyl quinidine, hydroquinidine 4-chlorobenzoate, L-(-)-α-amino-ε-caprolactam hydrochloride, D-(+)-α-amino-ε-caprolactam hydrochloride, (R)-(-)-1-amino-2-propanol, (S)-(+)-1-amino-2-propanol, chiral amino acids, brucine, cinchonine, cinchonidine, dihydro-cinchonidine, dihydrocinchonine, O-methylcinchonidine, cinchonan-6′,9-diol, cinchonan-9-ol, (9S)-(±)-10,11-dihydro-6′-methoxy-cinchonan-9-ol, 7′-(trifluoromethyl)-10,11-dihydrocinchonan-9-ol, cupreine, β-isocupreidine, euprocin, ethylhydrocupreine, (+)-dehydroabietylamine, (+)-dehydroabietylamine, (S)-(-)-N,α-dimethylbenzylamine, ephedrine, pseudoephedrine, (S)-α-methyl-2-pyridinemethanol (R)-α-methyl-2-pyridinemethanol, strychnine, 2R,4S,5R)-2-hydroxymethyl-5-ethylquinuclidine, (2S,4S,5R)-2-aminomethyl-5-ethylquinuclidine, (2R,5R)-(+)-5 -vinyl-2-quinuclidinemethanol, N-[3,5-bis(trifluoromethyl)phenyl]-N′-[(8a,9S)-10,11-dihydro-6′-methoxy-9-cinchonanyl]thiourea, N-[3,5-bis(trifluoromethyl)phenyl]-N′-[(9R)-6′-methoxy-9-cinchonanyl]thiourea, N-[3,5-bis(trifluoromethyl)phenyl]-N′-[(8a,9S)-6′-methoxy-9-cinchonanyl]thiourea, quinine ethyl carbonate, 9-acetoxyrubanone, (DHQD)2PHAL, (DHQ)2PHAL, (DHQD)2Pyr, (DHQ)2Pyr, (DHQD)2AQN and derivatives thereof.

In an alternative embodiment, the debenzylation of isopropyl ((benzyloxy)(phenoxy)phosphoryl)-L-alaninate is conducted in the presence of dihydroquinine to afford the dihydroquinine salt of isopropyl (hydroxy(phenoxy)phosphoryl)-L-alaninate:

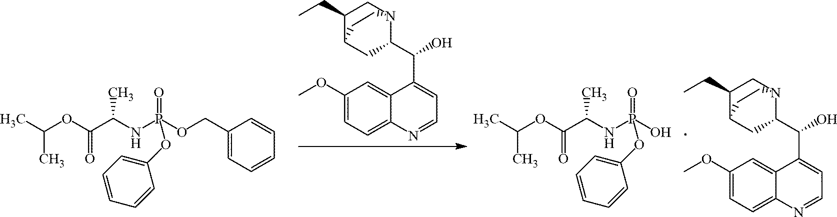 [SMILES: CC(C)OC(=O)[C@H](C)NP(=O)(OCc1ccccc1)Oc1ccccc1 ; CC[C@H]1C[N@]2CC[C@H]1C[C@H]2[C@H](O)c1ccnc2ccc(OC)cc12 ; CC(C)OC(=O)[C@H](C)NP(=O)(O)Oc1ccccc1 ; CC[C@H]1C[N@]2CC[C@H]1C[C@H]2[C@H](O)c1ccnc2ccc(OC)cc12]

In an alternative aspect, the process for the manufacture of the diastereomerically enriched Sₚ-phosphoramidate nucleotide of Formula XVI is provided:
(a) contacting Compound 2 with a compound of Formula XVII in the presence of a specified activator as described herein and base to afford a diastereomerically enriched Sₚ-phosphoramidate nucleotide of Formula XVI:

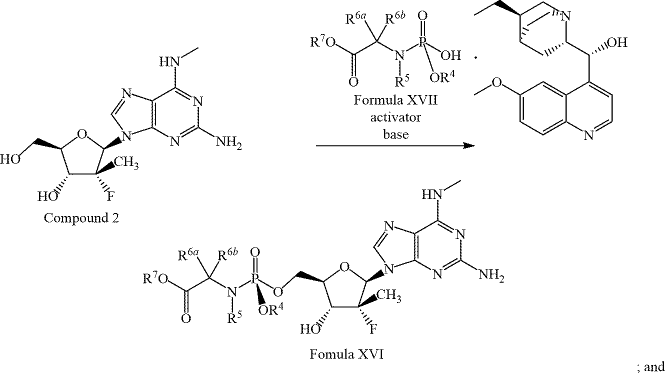

(b) further purifying the diastereomerically enriched Sₚ-phosphoramidate nucleotide of Formula XVI to afford the diastereomerically pure Sₚ-purine phosphoramidate nucleotide of Formula XVI wherein the diastereomerically purity is greater than about 90%, about 95%, or even about 99% or greater; and,
(c) optionally converting the compound of Formula XVI to a pharmaceutically acceptable salt of a compound of Formula XVI;
   wherein R², R⁴, R⁵, R^{6a}, R^{6b}, and R⁷ are as defined herein.

Non-limiting examples of a compound of Formula XVII include:

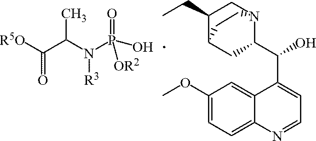

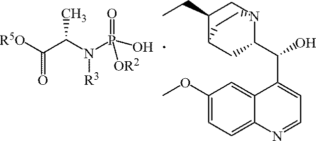

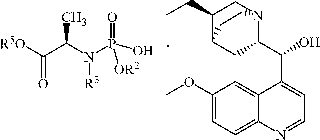

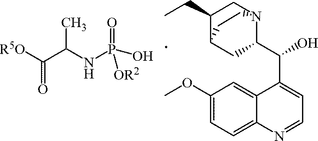

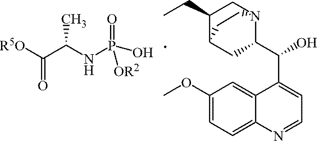

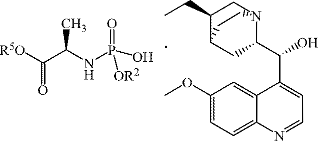

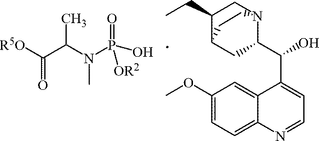

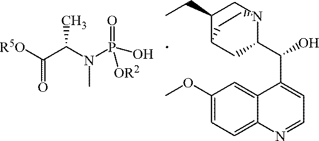

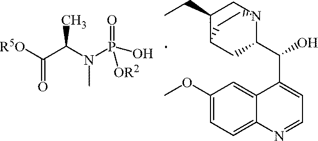

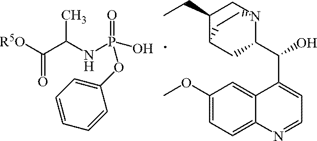

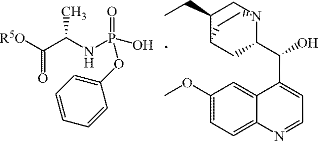

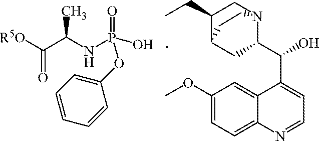

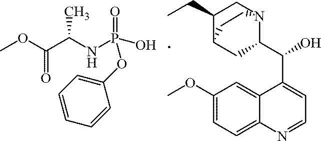

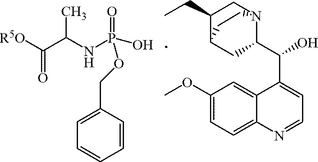

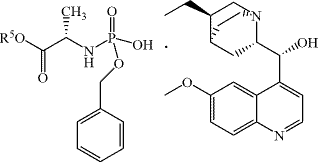

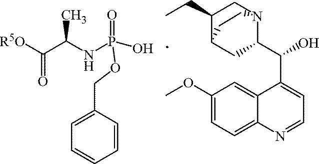

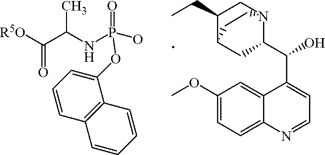

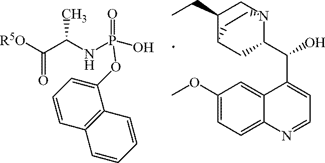

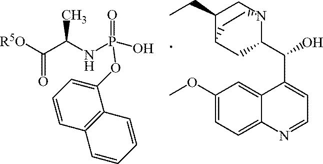

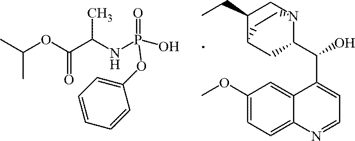 [SMILES: CC(C)OC(=O)C(C)NP(=O)(O)Oc1ccccc1 ; CC[C@H]1C[N@]2CC[C@H]1C[C@H]2[C@H](O)c1ccnc2ccc(OC)cc12]

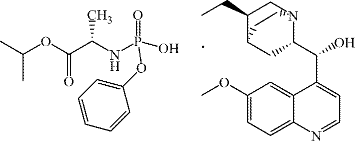 [SMILES: CC(C)OC(=O)[C@H](C)NP(=O)(O)Oc1ccccc1 ; CC[C@H]1C[N@]2CC[C@H]1C[C@H]2[C@H](O)c1ccnc2ccc(OC)cc12]

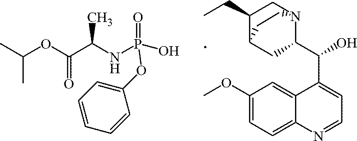 [SMILES: CC(C)OC(=O)[C@@H](C)NP(=O)(O)Oc1ccccc1 ; CC[C@H]1C[N@]2CC[C@H]1C[C@H]2[C@H](O)c1ccnc2ccc(OC)cc12]

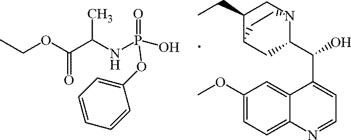 [SMILES: CCOC(=O)C(C)NP(=O)(O)Oc1ccccc1 ; CC[C@H]1C[N@]2CC[C@H]1C[C@H]2[C@H](O)c1ccnc2ccc(OC)cc12]

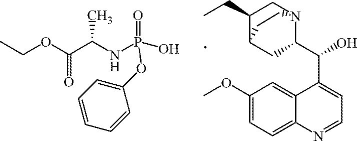 [SMILES: CCOC(=O)[C@H](C)NP(=O)(O)Oc1ccccc1 ; CC[C@H]1C[N@]2CC[C@H]1C[C@H]2[C@H](O)c1ccnc2ccc(OC)cc12]

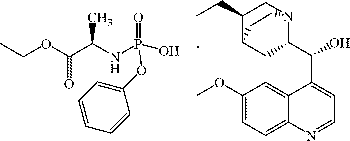 [SMILES: CCOC(=O)[C@@H](C)NP(=O)(O)Oc1ccccc1 ; CC[C@H]1C[N@]2CC[C@H]1C[C@H]2[C@H](O)c1ccnc2ccc(OC)cc12]

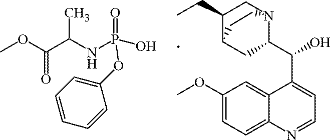 [SMILES: COC(=O)C(C)NP(=O)(O)Oc1ccccc1 ; CC[C@H]1C[N@]2CC[C@H]1C[C@H]2[C@H](O)c1ccnc2ccc(OC)cc12]

and

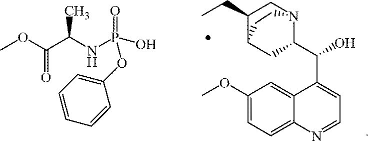

In one embodiment, the purification of the diastereomerically enriched Sₚ-phosphoramidate Compound 1 or the nucleotide of Formula VII, Formula IX, Formula XII, Formula XIV or Formula XVI to afford the corresponding diastereomerically pure Sₚ-purine phosphoramidate nucleotide is conducted via selective crystallization from an alkyl acetate, such as ethyl acetate, or a chlorinated solvent, such as dichloromethane, a ketone solvent, such as acetone, an aromatic solvent, such as toluene, or a mixture thereof. In one embodiment, the purification is conducted from crystallization from an alkyl acetate, chlorinated solvent, a ketone solvent, or a mixture thereof, with acetonitrile or an aliphatic hydrocarbon. In one embodiment, the purification is conducted from crystallization from an alkyl acetate, such as isopropyl acetate. In certain embodiments, the purification is conducted via selective crystallization from a mixture of ethyl acetate and toluene.

In one embodiment, the purification in step is the crystallization of the enriched mixture wherein the enriched mixture is dissolved in an organic solvent and then an anti-solvent is added dropwise to the above solution system wherein the organic solvent comprises a solvent selected from C₁₋₈ alcohols, C₂₋₈ ethers, C₃₋₇ ketones, C₃₋₇ esters, C₁₋₂ chlorocarbons, and C₂₋₇ nitriles and wherein the anti-solvent comprises at a solvent selected from C₅₋₁₂ saturated hydrocarbons, C₆₋₁₂ aromatic hydrocarbons, and petroleum ether. In one embodiment, the organic solvent is selected from ethyl acetate, tert-butyl methyl ether, isopropanol or tetrahydrofuran. In one embodiment, the anti-solvent is selected from petroleum ether or hexane.

In one embodiment, the purification of the diastereomerically enriched Sₚ-phosphoramidate nucleotide Compound 1 to afford the diastereomerically pure Sₚ-purine phosphoramidate nucleotide Compound 1 is conducted via crystallization from an alkyl acetate, such as ethyl acetate or isopropyl acetate, or a chlorinated solvent, such as dichloromethane, or a mixture thereof. In one embodiment, the purification is conducted via crystallization from an alkyl acetate, chlorinated solvent, or a mixture thereof, with acetonitrile or an aliphatic hydrocarbon.

The deprotection conditions of Formula VII, Formula IX, or Formula XII to afford Compound 1 are those generally known to the skilled artisan and are those described in Theodora W. Green, Protective Groups in Organic Synthesis, Third Edition, John Wiley & Sons (1999), which is incorporated by reference.

For example, when a protecting group selected from R^{1b}, R^{3a}, and R^{3b} is tert-butoxycarbonyl (Boc), the protecting group(s) can be removed via conditions described on pages 281 and 520-525, including the use of HCl in EtOAc; AcCl in MeOH; CF₃COOH in PhSH; and, TsOH in THF. In other embodiments, the protecting group(s) can be removed with DBU in MeOH.

When a protecting group selected from R^{1b}, R^{3a}, and R^{3b} is benzyloxycarbonyl (Cbz), the protecting group(s) can be removed via conditions described on pages 520-522, including: hydrogenation (H₂/Pd-C) and strongly acidic conditions (HBr, AcOH; 50% CF₃COOH; 70% HF, pyridine, CF₃SO₃H; FSO₃H, and CH₃SO₃H). In other embodiments, the protecting group(s) can be removed with DBU in MeOH.

When a protecting group selected from R^{1b}, R^{3a}, and R^{3b} is a substituted benzyl group, the protecting group(s) can be removed via conditions described on pages 86-101. For example, when R¹ is para-methoxybenzyl, deprotection conditions include DDQ (2,3-dichloro-5,6-dicyano-1,4-benzoquinone), CH₂Cl₂; and catalytic DDQ (2,3-dichloro-5,6-dicyano-1,4-benzoquinone), FeCl₃, CH₂Cl₂, H₂O.

When a protecting group selected from R^{1b}, R^{3a}, and R^{3b} is para-methoxybenzyloxymethyl, the protecting group(s) can be removed via conditions described on page 37, including 3:1 THF-6 M HCl.

The deprotection of the compound of Formula VI, Formula VIII, Formula XI, or Formula XIII wherein the R^{1a}, R², the R^{3a}, and/or the R^{3b} group need to be removed are also those generally known to a skilled artisan and described in Theodora W. Green, Protective Groups in Organic Synthesis, Third Edition, John Wiley & Sons (1999), which is incorporated by reference. For example, the R^{1a} group can be removed as discussed above and the R^{3a} and/or R^{3b} group can be removed as described in the text on pages 504-537 and 573-586. For example, when R^{3a} and/or R^{3b} is a methyl carbamate, R^{3a} and/or R^{3b} can be removed using HBr in AcOH and when R^{3a} and/or R^{3b} is a benzyl group, R^{3a} and/or R^{3b} can be removed using Pd/C in the presence of HCOOH.

An additional optional step includes:

(b) preparing the pharmaceutically acceptable salt form of the diastereomerically pure Sₚ-purine phosphoramidate nucleotide Compound 1.

In one embodiment, the pharmaceutically acceptable salt form of Compound 1 is the hemi-sulfate salt form, Compound 1-A:

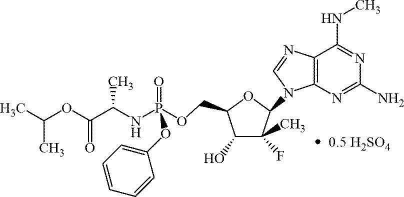

In one embodiment, Compound 1-A is prepared from Compound 1 by the dropwise addition of concentrated H₂SO₄ in MeOH and the filtration of the resulting precipitate. In an alternative embodiment, Compound 1-A is prepared from Compound 1 by the dropwise addition of concentrated H₂SO₄ in acetone and the filtration of the resulting precipitate.

Non-limiting examples of a compound of Formula XVI synthesized by the process of the present invention include:

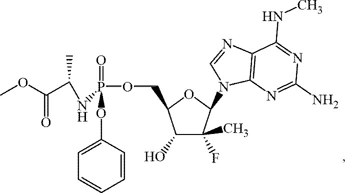

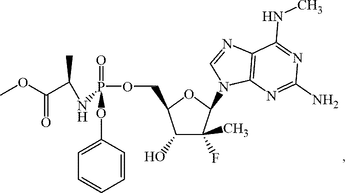

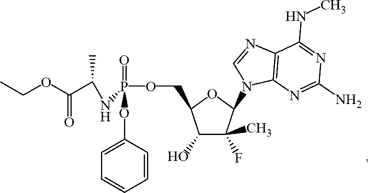

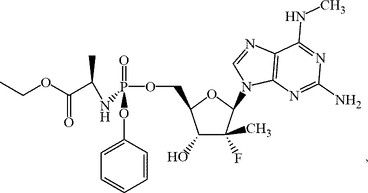

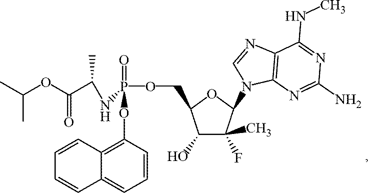

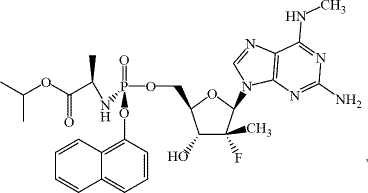

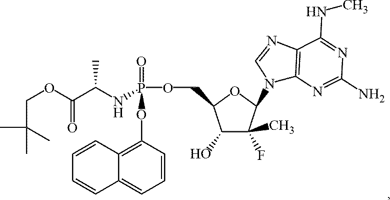

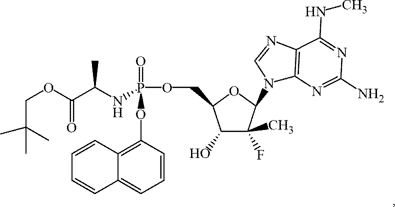

or a pharmaceutically acceptable salt thereof.

### EXAMPLES

### Example 1. Manufacture of the Dihydroquinine Salt of Isopropyl (hydroxy(phenoxy)phosphoryl)-L-alaninate

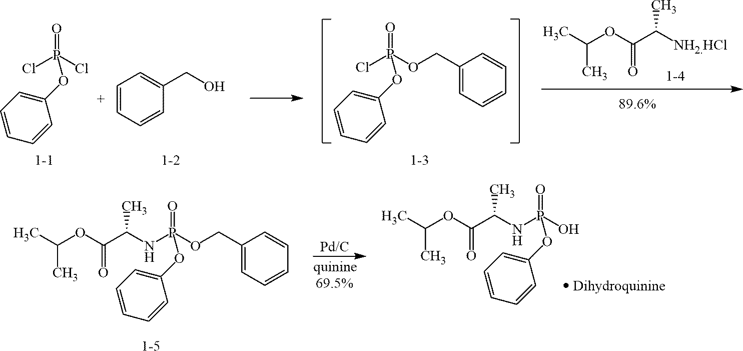

Phenyl dichlorophosphate (1-1, 150 g, 1.0 eq.) was added into 1300 mL of isopropyl acetate. The solution was cooled to -10° C. ± 5° C. and then a solution of benzyl alcohol (1-2, 80.6 g, 1.05 eq.) and Et₃N (86.3 g, 1.2 eq.) was added. The mixture was stirred for 3 hours at -10 ± 5° C. The end point of reaction was monitored by TLC.

L-Alanine isopropyl ester hydrochloride (1-4, 125 g, 1.05 eq.) and Et₃N (152 g, 2.1 eq.) were added at -10° C. ± 5° C. The reaction mixture was stirred at -10 ± 5° C. for 2 hours. The end point of reaction was monitored by TLC.

The reaction mixture was filtered, and the filter cake was washed with 20 mL of isopropyl acetate. The filtrate was washed with 1N HCl, water, and aqueous sodium bicarbonate. The separated organic layer was dried with anhydrous Na₂SO₄ and then concentrated to dryness under vacuum at 40° C.-50° C. to give 240 g of crude product 1-5 as a diastereomeric mixture (approximately, 1:1). (Pale yellow oil; yield: 89.6% mol/mol; HPLC purity: 83.4% by area; HPLC assay: 86.2% w/w). The product contained around 6%-7% residual benzyl alcohol. The crude 1-4 was used directly in the next step.

Compound 1-5 (135 g, 1.0 eq., 86.2% assay) and quinine (100 g, 1.0 eq.) were added into 650 mL of i-PrOH. After 5% Pd/C (19.2 g, 60% water by KF) was added, hydrogenation was performed at 20° C. -25° C. for 8 hours using a hydrogen bag in a closed system. After completion of reaction, the mixture was filtered through a Büchner funnel. The filtrate was concentrated under vacuum to remove the solvent.

To the above residue, 300 mL of TBME was added. The mixture was concentrated to remove the solvent under vacuum at 40° C.-45° C., and then this step was repeated with another 300 mL of MTBE. To the above, 600 mL of MTBE was added, and the mixture was stirred at 40° C.-45° C. for 1 hour and then stirred at 0° C.-5° C. for additional 1 hour. The mixture was filtered, and the filter cake was washed with 100 mL of MTBE. The cake was dried at 45° C. for 16 hours without vacuum to give 152 g of the dihydroquinine salt of isopropyl (hydroxy(phenoxy)phosphoryl)-L-alaninate (white solid; yield: 69.5% mol/mol; HPLC Purity: 97.91%).

### Example 2. General Manufacture of Compound 2

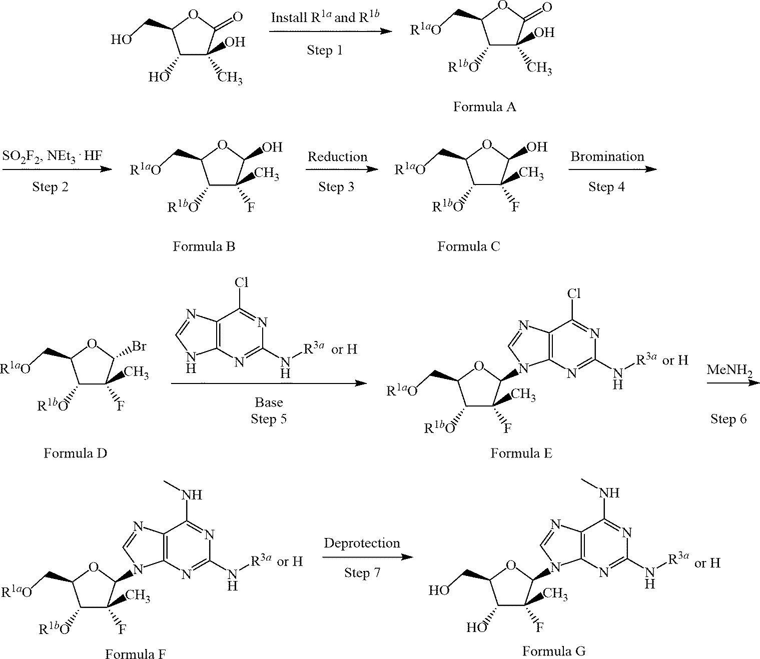

Step 1: (3S,4R,5R)-3,4-dihydroxy-5-(hydroxymethyl)-3-methyldihydrofuran-2(3H)-one is protected at the 3′- and 5′-positions with protecting groups R^{1a} and R^{1b} to afford a compound of Formula A.

Step 2: The hydroxy group is converted to fluorine with inversion of stereochemistry to afford a compound of Formula B.

Step 3: The ketone on the compound of Formula B is then reduced to afford a hydroxyl group to afford a compound of Formula C. In certain embodiments the reduction is stereoselective.

Step 4: The hydroxyl on the compound of Formula C is then displaced in a bromination reaction inverting the stereocenter to afford a compound of Formula D.

Step 5: The bromine on the compound of Formula D is then displaced by a nucleotide in a nucleophilic reaction to afford a compound of Formula E.

Step 6: The nucleotide on the compound of Formula E is then reacted with methyl amine to afford a compound of Formula F.

Step 7: Protecting groups R^{1a} and R^{1b} on the compound of Formula F are removed to afford a compound of Formula G.

### Example 3. Manufacture of Compound 2 Using Cbz Groups in R^{1a} and R^{1b} Position

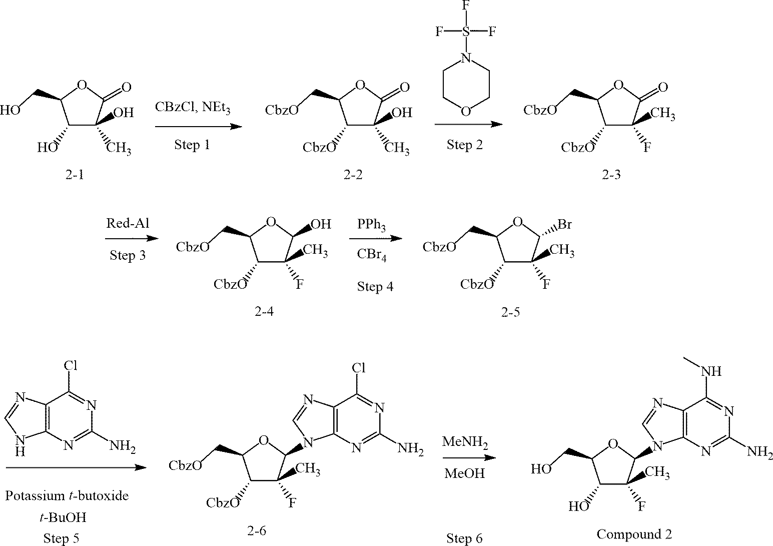

In Step 1, Compound 2-1 is dissolved in DCM and the reaction is cooled to 10° C. before benzyl chloroformate is added followed by NEt₃. The reaction is allowed to cool to room temperature and stir for 12-14 hours. Following appropriate work-up and purification conditions, Compound 2-2 is isolated. In Step 2, Compound 2-2 is dissolved in acetonitrile and cooled to -15 to 5° C. before Morpho DAST is added. The reaction is allowed to stir for 6 hours. Following appropriate work-up and purification conditions, Compound 2-3 is isolated. In Step 3, Compound 2-3 is dissolved in toluene and the reaction is cooled to 0 -10° C. before Red Al is added. Following appropriate work-up and purification conditions, Compound 2-4 is isolated as the diastereomer with (R)-stereochemistry at the hydroxyl position. In Step 4, Compound 2-4 is dissolved in acetonitrile and cooled to -15 to 5° C. before CBr₄ and PPh₃ are added. Following appropriate work-up and purification conditions, Compound 2-5 is isolated. In Step 5, Compound 2-5 is dissolved is acetonitrile and t-BuOH, t-BuOK, and 6-chloro-9H-purin-2-amine are added. The reaction is heated to 40 - 50° C. Following appropriate work-up and purification conditions, Compound 2-6 is isolated. In Step 6, Compound 2-6 is dissolved in MeOH and MeNH₂ is added. The reaction is heated to 20 - 30° C. Following appropriate work-up and purification conditions, Compound 2 is isolated.

In an alternative embodiment, Compound 2-4 is isolated as a mixture of diastereomers with regard to the stereochemistry at the hydroxyl group. Following isolation of the diastereomers, Compound 2-4 is dissolved in DCM and the reaction is cooled to 10° C. before acetyl chloride is added. The reaction is allowed to warm to room temperature and stir. Following appropriate work-up and purification conditions, Compound 2-5′ is isolated. In Step 5′, Compound 2-5′ is dissolved is acetonitrile and 6-chloro-9H-purin-2-amine and SnCl₄ are added. The reaction is warmed to 50 - 65° C. and allowed to stir until completion. Following appropriate work-up and purification conditions, Compound 2-6 is isolated. Similarly to above, in Step 6, Compound 2-6 is dissolved in MeOH and MeNH₂ is added. The reaction is heated to 20 - 30° C. Following appropriate work-up and purification conditions, Compound 2 is isolated:

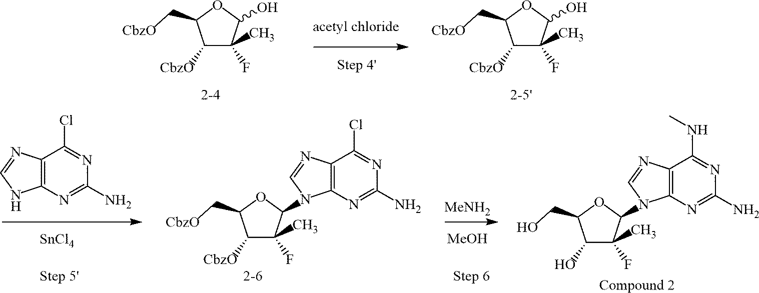

### Example 4. Manufacture of Compound 2 Using a Bridged Group in R^{1a} and R^{1b} Position

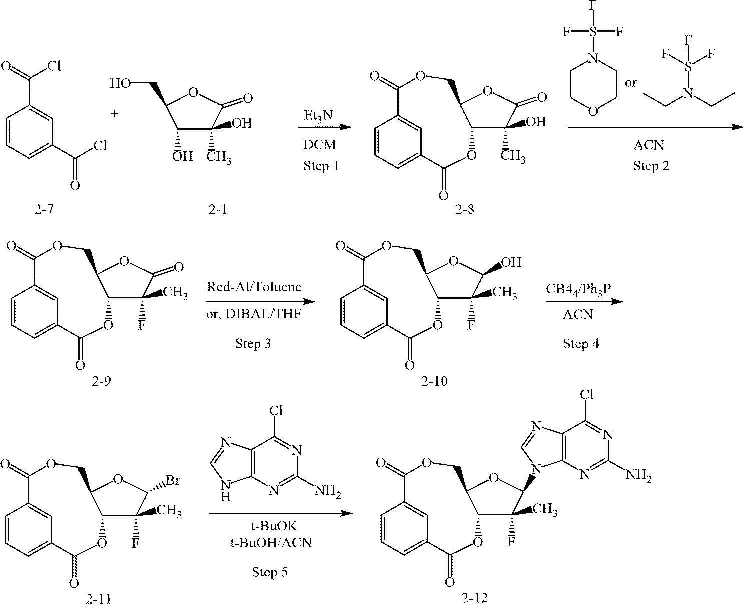

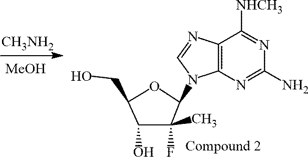

In Step 1, A solution of Compound 2-1 (1.0 mol) and 2.5 - 3.0 mol of triethylamine in DCM or THF can be added slowly to isophthaloyl dichloride (Compound 2-7, 1.0 mol) in DCM or THF under controlled temperature and the reaction mixture can be stirred until found complete by HPLC. Compound 2-8 can be isolated by extractive work up and purified by recrystallization from a suitable solvent (such as isopropyl alcohol, ethyl acetate, heptane, or a combination thereof). In Step 2, Compound 2-8 can be dissolved in acetonitrile and cooled to -15 to 5° C. before Morpho-DAST can be added. In an alternative embodiment, DAST is added instead of Morpho-DAST. The reaction can be allowed to stir for 6 - 8 hours. Following appropriate work-up and purification conditions, Compound 2-9 can be isolated. In Step 3, Compound 2-9 can be dissolved in toluene and the reaction can be cooled to 0 to -10° C. before Red-Al is added. The reaction can be allowed to stir for 1 -2 hours. In an alternative embodiment, Compound 2-9 can be dissolved in THF and DIBAL can be added after the reaction is cooled to -30° C. In this embodiment, the reaction can be allowed to stir for 2 - 4 hours. Following appropriate work-up and purification conditions, Compound 2-10 can be isolated as the diastereomer with (R)-stereochemistry at the hydroxyl position. In Step 4, Compound 2-10 can be dissolved in acetonitrile and cooled to -15 to 5° C. before CBr₄ and PPh₃ are added and the reaction can be allowed for stir for 2 hours. Following appropriate work-up and purification conditions, Compound 2-11 can be isolated. In Step 5, Compound 2-11 can be dissolved is acetonitrile and t-BuOH, t-BuOK, and 6-chloro-9H-purin-2-amine can be added. The reaction can be heated to 40 - 50° C. Following appropriate work-up and purification conditions, Compound 2-12 can be isolated. In Step 6, Compound 2-12 can be dissolved in MeOH and an excess of MeNH₂ can be added. The reaction is heated to 20 - 30 C and can be allowed to stir for 8 hours. Following appropriate work-up and purification conditions, Compound 2 can be isolated.

### Example 5. Manufacture of Compound 2 Using —C(O)OCH₃ Groups in R^{1a} and R^{1b} Position

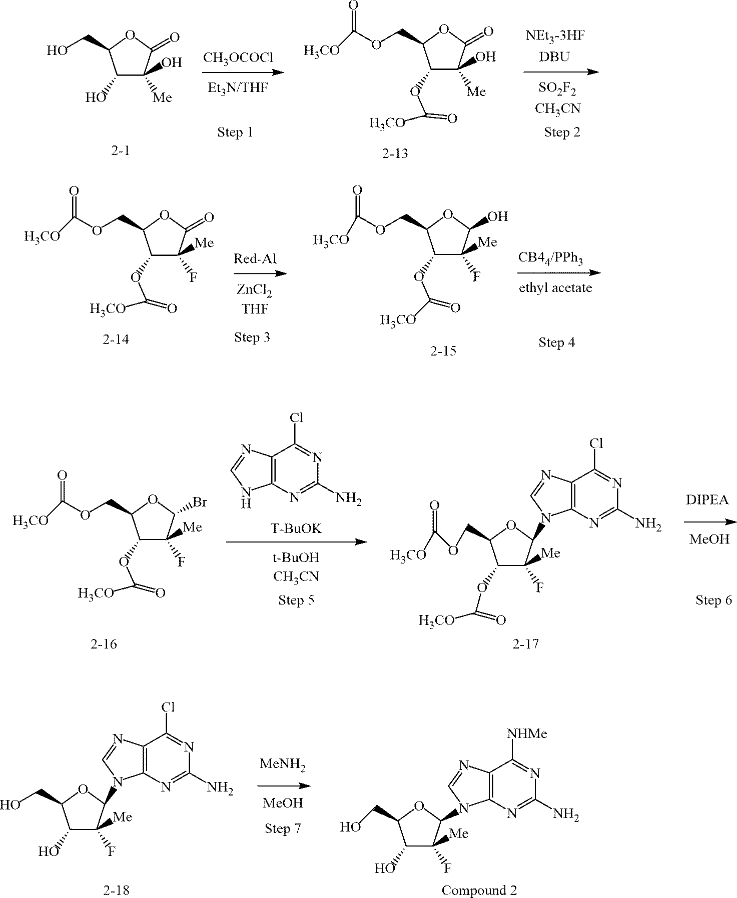

In Step 1, Compound 2-1 is dissolved in THF and methyl chloroformate and NEt₃ are added. The reaction is allowed to cool to room temperature and stir until complete. Following appropriate work-up and purification conditions, Compound 2-13 is isolated. In Step 2, Compound 2-13 is dissolved in acetonitrile and cooled before sulfonyl fluoride (SO₂F₂), triethylamine trifluoride (NEt₃3HF) and DBU are added. The reaction is allowed to stir until complete. Following appropriate work-up and purification conditions, Compound 2-14 is isolated. In Step 3, Compound 2-14 is dissolved in THF and the reaction is cooled before Red-Al and ZnCl₂ are added. Following appropriate work-up and purification conditions, Compound 2-15 is isolated as the diastereomer with (R)-stereochemistry at the hydroxyl position. In Step 4, Compound 2-15 is dissolved in ethyl acetate and cooled before CBr₄ and PPh₃ are added. Following appropriate work-up and purification conditions, Compound 2-16 is isolated. In Step 5, Compound 2-16 is dissolved is acetonitrile and t-BuOH, t-BuOK, and 6-chloro-9H-purin-2-amine are added. The reaction is heated to 40 - 50° C. Following appropriate work-up and purification conditions, Compound 2-17 is isolated. In Step 6, Compound 2-17 is dissolved in MeOH and DIPEA is added. The reaction is allowed to stir until complete. Following appropriate work-up and purification conditions, Compound 2-18 is isolated. In Step 7, Compound 2-18 is dissolved in MeOH and MeNH₂ is added. The reaction is heated to 20 - 30° C. Following appropriate work-up and purification conditions, Compound 2 is isolated.

### Example 6: Manufacture of Compound 2 Using —C(O)OC₁₆H₃₃ Groups in R^{1a} and R^{1b} Position

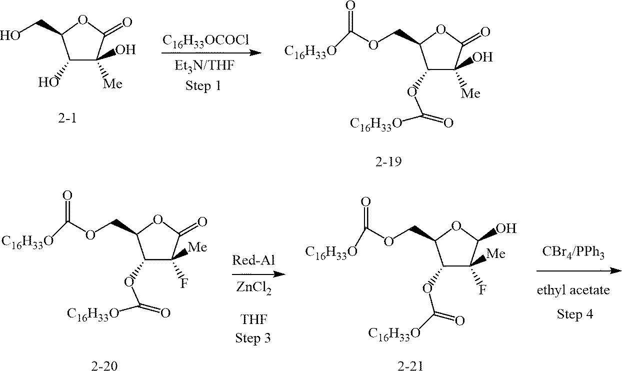

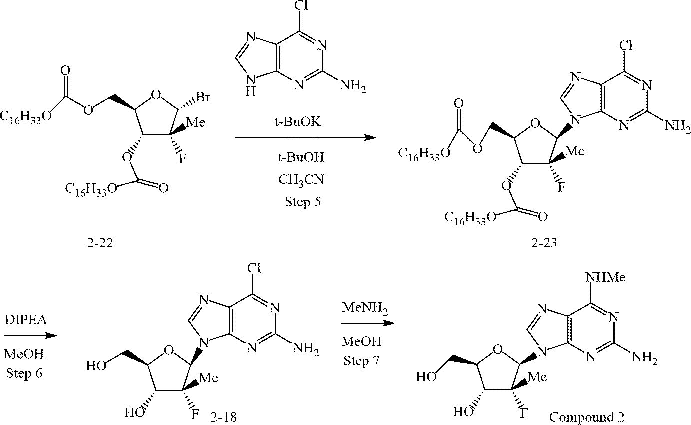

Step 1: Preparation of Compound 2-19: The lactone Compound 2-1 (10 g, 1.0 eq.) and triethylamine (12.3 g, 2.2 eq.) were dissolved in THF (100 mL). Then the solution was cooled to around -10-0° C. and hexadecyl carbonate chloride (34 g, 2.0 eq.) diluted with THF (20 mL) was slowly added to the reaction mixture at around -10-0° C. within 2 hours. After stirring for 6 hours, the reaction was completed as monitored by TLC. Then the formed triethylamine hydrochloride was removed by filtration and the solid was washed with THF (50 mL). The combined filtration was concentrated to remove THF. Then dichloromethane (200 mL) and water (100 mL) were added with stirring. After separating the phases, the solvent was removed by concentration. Heptane (300 mL) was charged to the residue, and the mixture was heated to 55° C. to afford a clear solution. After the solution was cooled to 20-25° C., the solid precipitated from the mixture. After stirring for 2 hours at 20-25° C., the product was filtered, washed with heptane (10 mL) and dried at 50° C. for 10 hours to afford Compound 2-19 (25 g) in 64% yield. ¹H NMR (400 MHz, DMSO-d₆) δ 5.18(d, 1H), 4.56 (dd, 1H), 4.46 - 4.44 (m, 1H), 4.37 (dd, 1H), 4.20 - 4.15 (m, 4H), 3.22 (brd, 1H), 1.69 - 1.65 (m, 4H), 1.47 (s, 3H), 1.25 (m, 52H). 0.88 (m, 6H).

Step 2: Preparation of Compound 2-20: NEt₃-3HF (3.44 g, 1.5 eq.) and DBU (6.6 g, 3 eq.) were dissolved in dichloromethane (100 mL) and the mixture was cooled to below 10° C. Compound 2-19 (10 g, 1.0 eq.) diluted with dichloromethane (20 mL) was dropped with bubbling of the gas of SO₂F₂. After the reaction was complete as monitored by TLC (6 hours), water (100 mL) was added to quench the reaction. DCM (100 mL) was charged to the mixture with stirring. Then the separated phase was concentrated and acetonitrile (100 mL) was added with heating (55-60° C.) to afford a clear solution. After cooling and stirring for 2 hours at 20-25° C., the product was filtered, washed by acetonitrile (10 mL), and dried at 50° C. for 6 hours to afford Compound 2-20 (9 g) in 90% yield. ¹H NMR (400 MHz, DMSO-d₆) δ 5.08 (dd, 1H), 4.72 - 4.71 (m, 1H), 4.58 (dd, 1H), 4.36 (dd, 4H), 4.27 - 4.13 (m, 4H), 1.76 - 1.57 (m, 7H), 1.26 (m, 52H). 0.89 (m, 6H).

Step 3: Preparation of Compound 2-21: Red-Al (6.3 mL, 1.5 eq. 70% solution in toluene) was added dropwise into anhydrous ZnCl₂ (2.9 g, 1.5 eq) solution in THF at -20 to -10° C. The mixture was stirred for 30 minutes at this temperature. Compound 2-20 (10 g, 14 mmol) was dissolved in THF (100 mL). The prepared above Red-Al-ZnCl₂ solution was added dropwise into the reaction under -20° C. and the reaction was stirred at -15 to -5° C. for 3-4 hours. Then the reaction mixture was poured into 5% HOAc in water (100 mL). After extraction using 100 mL of ethyl acetate, the solvent was removed by concentration to afford the crude product as a solid. Heptane (40 mL) was charged with heating to 55° C. to dissolve the solid. After cooling and stirring for 2 hours at 0-10° C., the product was filtered, washed by heptane (10 mL), and dried at 40° C. for 12 hours to afford Compound 2-21 (7.6 g) in 75% yield. ¹H NMR (400 MHz, DMSO-d₆) δ 5.25 (dd, 1H), 5.51 (dd, 1H), 4.47 - 4.44 (m, 1H), 4.35 - 4.30 (m, 1H), 4.20 - 4.13 (m, 4H), 3.52 (brd, 1H), 1.70 - 1.67 (m, 4H), 1.55- 1.50 (m, 3H), 1.38 - 1.27 (m, 52H). 0.90 (m, 6H).

Step 4: Preparation of Compound 2-22: Compound 2-21 (5.5 g, 7.82 mmol, 1.0 eq) was dissolved in DCM (30 mL) and the mixture was cooled to 0-10° C. under N₂ atmosphere. PPh₃ (5.1 g, 19.44 mmol, 2.5 eq) was added in the solution at 0-10° C. and CBr₄ (5.2 g, 15.68 mmol, 2.0 eq) was added portion-wise. The reaction was stirred at 5-10° C. for 1 hour at which point TLC monitoring showed the lactol was consumed completely. MeOH (60 mL) was added dropwise slowly in the mixture at 20-30° C. The mixture was stirred at 20-30° C. for 1 hour to remove OPPh₃ and other impurities (not including the α/β isomers). White solid was obtained by filtration and dried in oven at 35° C. for 3 hours to afford 4.5 g of Compound 2-22 in 75% yield (ratio of α/β isomers was around 3-4:1). ¹H NMR (400 MHz, DMSO-d₆) δ 6.33 (dd, 1H), 4.84 (dd, 1H), 4.64 -4.58 (m, 1H), 4.54 - 4.52 (m, 1H), 4.44 - 4.36 (m, 1H), 4.23 - 4.15 (m, 4H), 1.76 - 1.64 (m, 7H), 1.39 - 1.25 (m, 52H), 0.90 (m, 6H).

Step 5: Preparation of Compound 2-23: Cl-Purine (2.65 g, 3 eq) and t-BuOK (1.8 g, 16.04 mmol, 3.0 eq.) were added in ^{t}BuOH (40 mL). The reaction was kept at 55-60° C. for 1 hour. Compound 2-22 (4.0 g, 5.22 mmol, 1.0 eq.) in MeCN (60 mL) was added and the reaction was kept at 55-60° C. overnight. TLC showed the bromo-sugar was consumed completely. The reaction mixture was concentrated to remove most of the solvents and then ethyl acetate was added and the solid was removed. The solution was neutralized with 1N HCl. The organic phase was concentrated to afford a thick oil and methanol (30 mL) was added. After stirring for 1 hour, the solid precipitated and was filtered and dried to afford 3.8 g crude product. Then the crude product was dissolved in ethyl acetate (50 mL) and charged with charcoal (1 g). After filtration and concentration, the residual was crystallized from methanol (20 mL) to afford pure Compound 2-23 (2.5 g, 56% yield). The α-isomer was removed as shown by TLC. ¹H NMR (400 MHz, DMSO-d₆) δ 8.01 (s, 1H), 6.14 (d, 1H), 5.75 (dd, 1H), 5.52 (brd, 2H), 4.69 (d, 1H), 4.49 - 4.41 (m, 2H), 4.21 - 4.10 (m, 4H), 1.70 - 1.66 (m, 3H), 1.36 - 1.25 (m, 56H), 0.89 (m, 6H).

Step 6: Preparation of Compound 2-18: Compound 2-23 (5 g 5.85 mmol, 1.0 eq.) was dissolved in MeOH (30 mL). DIPEA (1.51 g, 11.7 mmol, 2.0 eq.) was added dropwise in the reaction. The reaction was then warmed to 50° C. and stirred at this temperature for 18 hours. TLC showed complete conversion. The reaction was concentrated and redissolved in MTBE (25 mL) and concentrated again. Then MTBE (25 mL) was added and the mixture was stirred as a slurry at room temperature for 30 minutes. The solid was filtered and drip washed by MTBE (5 mL). The collected solid was dried at 50° C. in oven and 1.74 g of white solid powder was obtained as Compound 2-18 in 93% yield.

Step 7: Preparation of Compound 2: Compound 2-18 (1.5 g, 1.0 eq.) was dissolved in THF (15 mL). MeNH₂ aq. (28%, 1.6 g, 3.0 eq.) was dropped into the solution. The reaction was stirred at 20-30° C. overnight and the starting material was consumed completely. To the reaction mixture was added a solution of NaHCO₃ (410 mg, 1.0 eq.) in H₂O (5 mL). After stirring for 10 minutes, the mixture was concentrated under reduced pressure. The residue was redissolved in EtOH (20 mL). The concentration-resolution was repeated twice and the residue was stirred in EtOH (20 mL). The mixture was filtered to remove salts and the filtrate was concentrated. The residue was dissolved in EtOAc and the solution was concentrated. Then EtOAc (10 mL) was added and the solution was stirred at 50° C. for 0.5 hour and the solids precipitated. The mixture was cooled slowly with stirring overnight and the solid will filtered and dried at 55° C. for 7 hours to afford 1.1 g of Compound 2 in 75% yield and with a purity of 97.36% (Area by HPLC).

### Example 7: Manufacture of Compound 2 Using —C(O)NHPhGroups in R^{1a} and R^{1b} Position

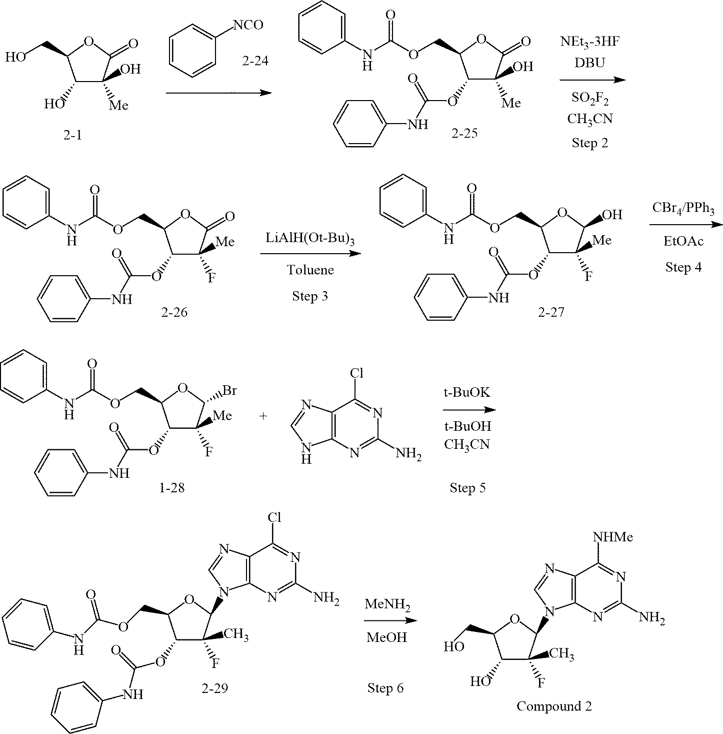

In Step 1, Compound 2-1 is dissolved in THF and brought to 0° C. using an ice bath before Compound 2-24 and NEt₃ are added. The reaction is allowed to cool to room temperature and stir until complete. Following appropriate work-up and purification conditions, Compound 2-25 is isolated. In Step 2, NEt₃-3HF and DBU are dissolved with acetonitrile and the mixture is cooled to 0-10° C. Compound 2-25 diluted with acetonitrile is dropped with bubbling of the gas of SO₂F₂. The reaction is allowed to stir until complete. Following appropriate work-up and purification conditions, Compound 2-26 is isolated. In Step 3, Compound 2-26 is dissolved in toluene and the reaction is cooled before LiAlH(Ot-Bu)₃ is added. Following appropriate work-up and purification conditions, Compound 2-27 is isolated as the diastereomer with (R)-stereochemistry at the hydroxyl position. In Step 4, Compound 2-27 is dissolved in ethyl acetate and cooled before CBr₄ and PPh₃ are added. Following appropriate work-up and purification conditions, Compound 2-28 is isolated. In Step 5, Compound 2-28 is dissolved is acetonitrile and t-BuOH, t-BuOK, and 6-chloro-9H-purin-2-amine are added. The reaction is heated to 40 - 50° C. Following appropriate work-up and purification conditions, Compound 2-29 is isolated. In Step 6, Compound 2-29 is dissolved in MeOH and MeNH₂ is added. The reaction is heated to 20 - 30 C. Following appropriate work-up and purification conditions, Compound 2 is isolated.

### Example 8: Manufacture of Compound 2 Using —C(O)N(Ph)₂ Groups in R^{1a} and R^{1b} Position

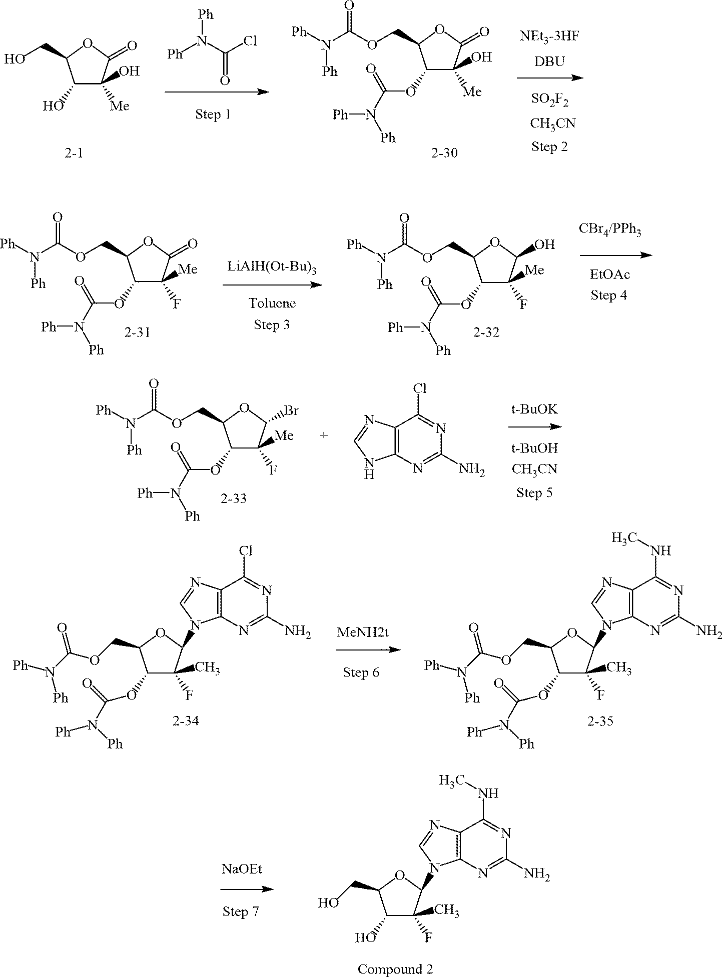

In Step 1, Compound 2-1 is dissolved in THF and brought to 0° C. using an ice bath before diphenylcarbamic chloride is added. The reaction is allowed to cool to room temperature and stir until complete. Following appropriate work-up and purification conditions, Compound 2-30 is isolated. In Step 2, NEt₃-3HF and DBU are dissolved with acetonitrile and the mixture is cooled to 0-10° C. Compound 2-30 diluted with acetonitrile is dropped with bubbling of the gas of SO₂F₂. The reaction is allowed to stir until complete. Following appropriate work-up and purification conditions, Compound 2-31 is isolated. In an alternative reaction, the fluorination is conducted with DAST. In Step 3, Compound 2-31 is dissolved in toluene and the reaction is cooled before LiAlH(Ot-Bu)₃ is added. Following appropriate work-up and purification conditions, Compound 2-32 is isolated as the diastereomer with (R)-stereochemistry at the hydroxyl position. In Step 4, Compound 2-32 is dissolved in ethyl acetate and cooled before CBr₄ and PPh₃ are added. Following appropriate work-up and purification conditions, Compound 2-33 is isolated. In Step 5, Compound 2-33 is dissolved is acetonitrile and t-BuOH, t-BuOK, and 6-chloro-9H-purin-2-amine are added. The reaction is heated to 40 - 50° C. Following appropriate work-up and purification conditions, Compound 2-34 is isolated. In Step 6, Compound 2-34 is dissolved in MeOH and MeNH₂ is added. The reaction is heated to 20 - 30° C. Following appropriate work-up and purification conditions, Compound 2-35 is isolated. In Step 7, Compound 2-35 is dissolved in an appropriate solvent and NaOEt is added. Following appropriate work-up and purification conditions, Compound 2 is isolated.

### Example 9. Manufacture of Compound 1

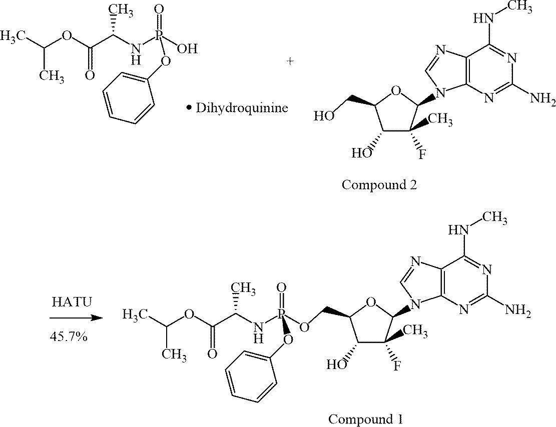

The dihydroquinine salt of isopropyl (hydroxy(phenoxy)phosphoryl)-L-alaninate (5.9 g, 1.5 eq.), Compound 2 (2.0 g, 1.0 eq), DIPEA (0.83 g, 1.0 eq), and HATU (3.65 g, 1.5 eq) were added into 100 mL of dichloromethane. The mixture was heated to 40° C. and stirred for 18 hours. The reaction was monitored by TLC and HPLC.

After the reaction was completed, the reaction mixture was cooled to room temperature, washed with 1N hydrochloric acid (100 mL x 2), water (100 mL x 2), and 5% aqueous sodium bicarbonate 15 mL x 1). The separated organic phase was dried with 2 g of anhydrous sodium sulfate, filtered, and concentrated at 40° C.-45° C. under vacuum to give a yellow oil.

Isopropyl acetate (10 mL of) was added. After stirring, the mixture was concentrated under vacuum. Then, 25 mL of isopropyl acetate was added. The mixture was heated to 45° C. to afford a clear solution. After stirring at room temperature for 2 hours, the solid precipitate was filtered and dried without vacuum at 45° C. for 15 hours to give 2.0 g of crude Compound 1 (yield: 53.8% mol/mol; HPLC purity: 93.1% by area (containing 3.7% of Rₚ-Compound 1).

The mixture of crude Compound 1 (2.0 g) and 15 mL of isopropyl acetate was heated to 80° C.-85° C. to afford a solution. The solution was cooled to 20° C.-25° C. and stirred for 1 hour. The precipitated solid was filtered, washed with isopropyl acetate (1 mL), and dried without vacuum at 50° C. for 16 hours to give 1.7 g of Compound 1 (yield: 45.7% mol/mol; HPLC purity: 98.99%). ¹H NMR, ¹⁹F NMR, and ³¹P NMR spectra confirmed the structure of Compound 1.

### Example 10. Alternative Manufacture of Compound 1 and Compound 1-A

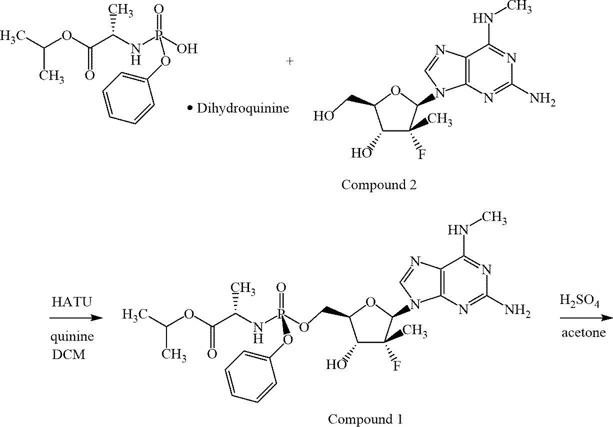

The dihydroquinine salt of isopropyl (hydroxy(phenoxy)phosphoryl)-L-alaninate, Compound 2, quinine, and HATU are added into dichloromethane. The mixture is heated and stirred until complete by TLC and HPLC. Following appropriate work-up and purification conditions, Compound 1 is isolated. Compound 1 is then dissolved in acetone and H₂SO₄ is added dropwise. The reaction is allowed to stir until complete. Following appropriate work-up and purification conditions, Compound 1-A is isolated.

### Example 11. Manufacture of Compound 2, Compound 1, and Compound 1-A

### Part A. Synthesis of the Dihydroquinine Salt of Isopropyl (hydroxy(phenoxy)phosphoryl)-L-alaninate

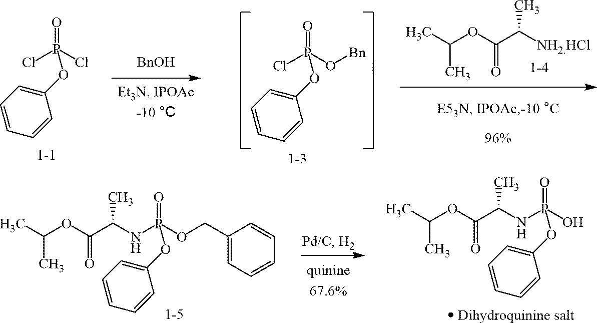

Phenyl dichlorophosphate 1-1 (175 g, 1.0 eq.) was added into 1750 mL of isopropyl acetate. The solution was cooled to -10±5° C. and then a solution of benzyl alcohol (95 g, 1.05 eq.) and Et₃N (105 g, 1.0 eq.) were added. The mixture was stirred for 3 hours at -10 ±5° C. to form the benzyl phenyl phosphorochloridate intermediate 1-3. L-Alanine isopropyl ester hydrochloride 1-4 (140 g, 1.0 eq.) and Et₃N (180 g, 2.1 eq.) were added at -10 ±5° C. The reaction mixture was stirred at -10 +5° C. for 2 hours. Then the solid in reaction mixture was filtered and the filter cake was washed with 100 mL of isopropyl acetate. The filtrate was washed with water (750 mL), 1N HCl (750 mL), and saturated aqueous sodium bicarbonate (750 mL) and water (750 mL). Charcoal (25 gram) was added to the separated organic phase. After stirring for 2 hours at 25-30° C., the mixture was filtered, and the cake was washed with 100 mL of isopropyl acetate. The combined filtrate was concentrated under vacuum at 40-50° C. to afford 300 g of crude product Compound 1-5. (Pale yellow oil; yield: 96% mol/mol; HPLC purity: 91.5% by area). ¹H NMR (400 MHz, Chloroform-d) δ 7.37 - 7.13 (m, 10H), 5.13 (t, J = 8.0 Hz, 2H), 5.00 - 4.95 (m, 1H), 3.95 - 3.92 (m, 1H), 3.60 (q, J = 8.0 Hz, 1H), 1.35 - 1.29 (m, 3H), 1.22 - 1.18 (m, 6H).

Compound 1-5 (300 g, 1.0 eq.) and quinine (250 g, 0.9 eq.) were added into 1750 mL of i-PrOH. After 5% wet Pd/C (45 g, 60% water by KF) was added, hydrogenation was performed at 20-25° C. for 20 hours. The mixture was filtered through a Büchner funnel. The filtrate was concentrated to dryness under vacuum. MTBE (1000 mL) was added. The mixture was concentrated to dryness under vacuum at 40-45° C., and then this step was repeated again. MTBE (1500 mL) was added and the mixture was stirred at 50° C. for 1 hour and then stirred at 0-5° C. for 1 hour. The mixture was filtered to give the crude product. Then, to the cake was added 1250ml of acetonitrile. The mixture was heated to reflux to give a clear solution. Then the solution was cooled to 0-10° C. and stirred for 3 hours at this temperature. The precipitated solid was filtered, washed with 100 ml of cold acetonitrile, and dried at 45° C. for 16 hours without vacuum to afford 330 g of the dihydroquinine salt of isopropyl (hydroxy(phenoxy)phosphoryl)-L-alaninate. (White solid; Yield of two steps: 65% mol/mol; HPLC Purity: 99.4% by area). ¹H NMR (400 MHz, DMSO-d₆) δ 12.56 (s, 1H), 8.75 (d, J = 4.5 Hz, 1H), 7.97 (d, J= 9.2 Hz, 1H), 7.65 (d, J = 4.4 Hz, 1H), 7.55 (d, J = 2.6 Hz, 1H), 7.42 (dd, J = 9.2, 2.6 Hz, 1H), 7.26 - 7.09 (m, 4H), 6.93 (t, J = 7.0 Hz, 1H), 6.56 (s, 1H), 6.06 (s, 1H), 4.80 (h, J= 6.2 Hz, 1H), 3.98 (s, 5H), 3.77 (q, J= 7.7 Hz, 1H), 3.59 (s, 1H), 3.49 (s, 1H), 3.04 (s, 1H), 2.82 (s, 1H), 2.09 - 1.89 (m, 3H), 1.75 (d, J = 34.0 Hz, 2H), 1.46 - 1.33 (m, 1H), 1.33 - 1.18 (m, 2H), 1.15 (d, J= 7.0 Hz, 3H), 1.12 - 1.09 (m, 6H), 0.76 (t, J = 7.3 Hz, 3H).

### Part B. Synthesis of Compound 2

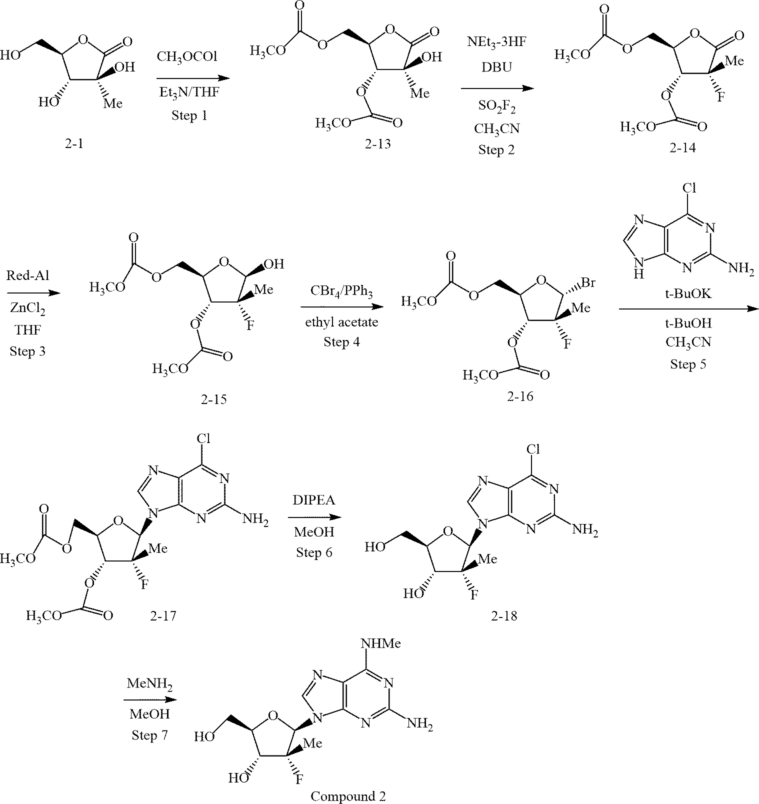

Step 1: Preparation of Compound 2-13: Compound 2-1 (20 g, 1.0 eq.) was dissolved in THF (100 mL) and the solution was cooled to -55~5° C. Then Et₃N (22.4 g, 2.0 eq.) and methyl chloroformate (21 g, 2.0 eq.) were simultaneously added dropwise into the reaction at -15~5° C. The addition was finished within 1 hour, and the reaction was stirred for another 3 hours at this temperature. After a filtration of the formed triethylamine hydrochloride, the filtrate was concentrated at 40-45° C. Then toluene (60 mL) and H₂O (15 mL) were added with stirring for 5 hours at 0-10° C. The precipitated solid was filtered, washed with water (20 mL), and dried at 45-50° C. for 8 hours to afford Compound 2-13 as a white solid (24 g) in 78% yield. ¹H NMR (400 MHz, DMSO-d₆) δ 5.23(d, 1H), 4.58 (dd, 1H), 4.49 - 4.45 (m, 1H), 4.42 - 4.37 (m, 1H), 3.87 (s, 3H), 3.83 (s, 3H), 3.02 (brd, 1H), 1.46 (s, 3H).

Step 2: Preparation of Compound 2-14: NEt₃-3HF (6.1 g, 1.5 eq.) and the DBU (11.5 g, 3eq.) were dissolved with acetonitrile (35 mL) and the mixture was cooled to 0-10° C. Compound 2-13 (7 g, 1.0 eq.) diluted with acetonitrile (14 mL) was dropped with bubbling of the gas of SO₂F₂. After the reaction was complete as monitored by TLC (2 hours), water (70 mL) was added to quench this reaction. DCM (70 mL) was charged to the mixture with stirring. Then the separated phase was concentrated, and i-propanol (100 mL) was added with heating (55-60° C.) to afford a clear solution. After cooling and stirring for 2 hours at 0-10° C., the product was filtered, washed by i-propanol (5 mL), and dried at 50° C. to afford Compound 2-14 (6 g) in 85% yield. ¹H NMR (400 MHz, DMSO-d₆) δ 5.09 (dd, 1H), 4.71 (m, 1H), 4.59 (dd, 1H), 4.37 (dd, 1H), 3.88 (s, 3H), 3.82 (s, 3H), 1.76 (d, 3H).

Step 3: Preparation of Compound 2-15: Anhydrous ZnCl₂ (14.6 g 0.107 mol, 1.5 eq.) was added to THF (200 mL) at 20-25° C. After stirring for 30 minutes, Compound 2-14 (20 g, 0.07 mol, 1.0 eq.) was added to the solution. The mixture was cooled to -20 ~ -10° C. and Red-Al (31 g, 0.107 mol, 1.5 eq., 70% solution in toluene) was added dropwise at this temperature within 2 hours. After the reaction was stirred for another 1 hour, the starting material was consumed completely monitored by TLC. The reaction mixture was poured into 10% HOAc in water (200 mL) at below 10° C. Then toluene (200 mL) was charged and the phases were separated. The aqueous phase was extracted once with toluene (100 mL). The combined organic phase was washed with sodium bicarbonate solution (100 mL, 5%). After the organic solution was concentrated to one volume of around 30 mL, the mixture was cooled to 0-10° C. and stirred for 2 hours at 0-10° C. The precipitated solid was filtered, washed by toluene (5 mL), and dried at 40° C. for 12 hours to afford Compound 2-15 (15.3 g) in 75% yield. ¹H NMR (400 MHz, DMSO-d₆) δ 7.23 (d, 1H), 4.97 (m, 1H), 4.84 (dd, 1H), 4.38 (m, 1H), 4.12 (m, 1H), 3.77 (s, 3H), 3.74 (s, 3H), 1.37 (d, 3H).

Step 4: Preparation of Compound 2-16: Compound 2-15 (12 g, 1.0 eq, α/β ratio of approximately 13/87) was dissolved in ethyl acetate (60 mL) and the mixture was cooled to 0-10° C. under N₂ atmosphere. PPh₃ (17.9 g, 1.6 eq) was added in the solution at 0-10° C. and CBr₄ (21.1 g, 1.5eq) was added portion-wise. The reaction was stirred at 5-10° C. for 2 hours at which point TLC monitoring showed the complete consumption of the lactol. The precipitated solid was removed by filtration, the solid was washed with ethyl acetate (2*5 mL). Then the filtrate was washed with sodium bicarbonate solution (20 mL, 5%), and dried by anhydrous sodium sulfate. Heptane (250 mL) was added to the ethyl acetate solution and the solid was precipitated. The solid was filtered, washed with heptane (10 mL) and dried at 40° C. for 4 hours to afford 13 g of Compound 2-16. By HPLC determination, the yield was 62% by calculation. The analytical sample was prepared by column chromatography with an eluent of heptane/ethyl acetate= 20:1. ¹H NMR (400 MHz, DMSO-d₆) δ 6.25 (s, 1H), 4.84 (dd, 1H), 4.63 - 4.61 (m, 1H), 4.55 (dd, 1H), 4.42 (dd, 1H), 3.88 (s, 3H), 3.83 (s, 3H), 1.69 (d, 3H).

Step 5: Preparation of Compound 2-17: Cl-Purine (6.4 g, 3 eq.) and t-BuOK (4.2 g, 3.0 eq.) were added into t-BuOH (45 mL). The reaction was kept at 55-60° C. for 1 hour. Then Compound 2-16 (6.0 g, 72% assay, 1.0 eq.) solution in acetonitrile (65 mL) was added and the reaction was kept at 55-60° C. overnight. TLC showed the bromo-sugar was completely consumed. The reaction mixture was cooled to 0-10° C. and neutralized with hydrochloric acid (37%, 1.1 mL). Then the solid was removed by filtration and the filtrate was concentrated. Ethyl acetate (20 mL) was added and the resulted solid was removed again by filtration. Then the solution was washed with water, dried by anhydrous sodium sulfate and concentrated. The residual was purified by column chromatography with an eluent of dichlorometahne/methanol = 30:1 to afford Compound 2-17 (2.82 g, 52% yield). ¹H NMR (400 MHz, DMSO-d₆) δ 8.29 (s, 1H), 7.09 (s, 2H), 6.27 (d, 1H), 5.69 (dd, 1H), 4.60 (dd, 1H), 4.53 (dd, 1H), 4.43 - 4.39 (m, 1H) 3.81 (s, 3H), 3.73 (s, 3H), 1.27 (d, 3H).

Step 6: Preparation of Compound 2-18: Compound 2-17 (2 g, 1.0 eq.) and diisopropyl ethylamine (1.2 g, 2.0 eq.) were added into methanol (10 mL). Then the mixture was warmed to 45-50° C. overnight. After the TLC showed the starting material was consumed completely, the reaction mixture was cooled to 0-10° C. with stirring. The precipitated solid was collected by filtration, washed with methanol (2 mL), and dried at 50° C. for 7 hours to afford 1.14 g of Compound 2-18 with a yield of 78.1%. ¹H NMR (400 MHz, DMSO-d₆) δ 8.44 (s, 1H), 7.09 (s, 1H), 6.10 (d, 1H), 5.70 (d, 1H), 5.26 (t, 1H), 4.22 - 4.16 (m, 1H), 3.95 - 3.84 (m, 2H), 3.72 - 3.71 (m, 1H), 1.15 (d, 3H).

Step 7: Preparation of Compound 2: MeNH₂ aq. (28%, 0.85 g, 3.0 eq.) was dropped into the solution of Compound 2-18 (1.5 g, 1.0 eq.) in THF (12 mL). The reaction was stirred at 20-30° C. for 7 hours at which point the starting material was consumed completely. To the reaction mixture was added a solution of NaHCO₃ (220 mg, 1.0 eq.) in H₂O (3 mL). After stirring for 10 minutes, the mixture was concentrated under reduced pressure. The residue was redissolved in EtOH (15 mL). The concentration-resolution was repeated twice and the residue was stirred in EtOH (20 mL). The mixture was filtered to remove salts and the filtrate was concentrated. The residue was dissolved in EtOAc and the solution was concentrated. Then EtOAc (10 mL) was added and the solution was stirred at 50° C. for 0.5 hours and the solids precipitated. The mixture was cooled to 0-5° C. for 3 hours and the solid will filtered and dried at 55° C. for 8 hours to afford 0.68 g of Compound 2 in 86% yield. ¹H NMR (400 MHz, DMSO-d₆) δ 7.98 (s, 1H), 7.29 (s, 2H), 6.03 - 5.97 (m, 3H), 5.64 (d, 1H), 5.26 (s, 1H), 4.23 - 4.17 (m, 1H), 3.91 - 3.83 (m, 2H), 3.70 (m, 1H), 2.89 (s, 3H), 1.23 (d, 3H).

### Part C: Synthesis of Compound 1 and Compound 1-A

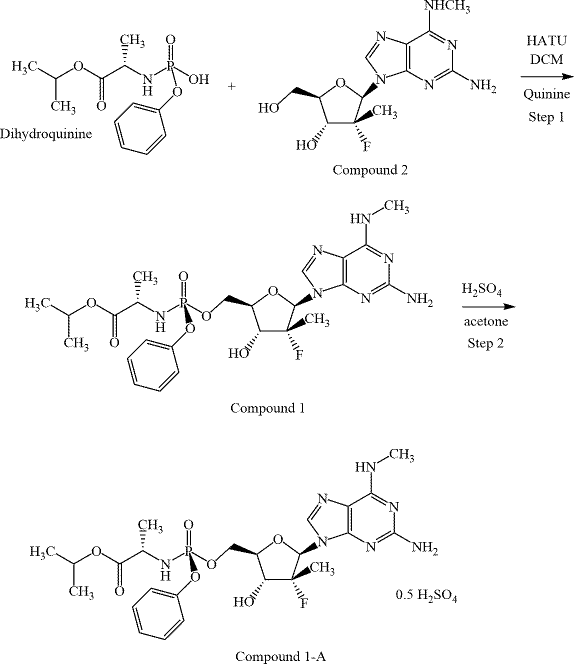

Step 1: Preparation of Compound 1: The dihydroquinine salt of isopropyl (hydroxy(phenoxy)phosphoryl)-L-alaninate: (295 g, 1.5eq.), Compound 2 (100 g, 1.0 eq.), DIPEA (41.5 g, 1.0 eq.) and HATU (182.5 g, 1.5 eq.) were added into 1500 mL ofdichloromethane. The mixture was heated to 40° C. and stirred for 18 hours. The reaction was monitored by TLC and HPLC.

Work-up, stage 1: After the reaction was complete, the reaction mixture was cooled to 0-10° C. 6N hydrochloric acid (400 mL) was slowly added at 0-10° C. and most of the dihydroquinine hydrochloride precipitated. The precipitated solid was filtered and the filtrate was washed with 2N HCl (500 mL), 5% aqueous sodium bicarbonate (500 mL) and water (500 mL). The separated organic phase was concentrated at 40-45° C. under vacuum to afford yellow oil.

Stage 2: Isopropyl acetate (400 mL) and water (5000 mL) were added to dissolve the above oil. Then 2N HCl was added to adjust the pH=4 to form the salt. After standing for 10 minutes, the phases were well separated. Many impurities entered the upper organic phase, and the hydrochloride of Compound 1 stayed in the down aqueous phase. Then the aqueous phase was adjusted to pH=8 and washed with dichloromethane (1250 mL*2) to extract Compound 1 to the organic phase. The combined organic phases were washed water (500 mL) and then charcoal (10 g) was added. After stirring for 2 hours at 25-30° C., the mixture was filtered, and the cake was washed with 20 mL of dichloromethane. The dichloromethane was removed by concentration and 200 mL of isopropyl acetate was added to the concentration again to afford yellow oil product.

Stage 3: Isopropyl acetate (600 mL) was added. The mixture was heated to 50° C. to afford a clear solution. After stirring at room temperature for 2 hours, the precipitation was filtered to afford an off-white solid of crude Compound 1.

Stage 4 (Re-crystallization): The above crude Compound 1 and 600 mL of isopropyl acetate was heated to around 50° C. to afford a solution. The solution was cooled to 20-25° C. and stirred for 2 hours. The precipitated solid was filtered, washed with isopropyl acetate (50 mL) and dried without vacuum at 45-50° C. for 16 hours to afford 112.5 g of Compound 1. (Yield: 60.5% mol/mol; HPLC purity: 99.1% by area). ¹H NMR (400 MHz, DMSO-d₆) δ 7.81 (s, 1H), 7.38 -7.34 (m, 3H), 7.24 - 7.18 (m, 3H), 6.07 - 6.00 (m, 4H), 5.76 (d, J = 6.8 Hz, 1H), 4.87 - 4.78 (m, 1H), 4.44 - 4.40 (m, 1H), 4.34 - 4.29 (m, 1H), 4.10 - 4.06 (m, 1H), 3.81 - 3.78 (m, 1H), 2.88 (s, 3H), 1.22 - 1.12 (m, 12H).

Step 2: Preparation of Compound 2: Compound 1 (14 g) was added to acetone (180 mL) and the mixture was stirred at 20-30° C. to afford a solution. Then sulfuric acid (1.12 g, 0.475 eq.) was slowly added at 15-20° C. (within 30 min) and the solids gradually precipitated. The mixture was stirred at 15-20° C. for 30 minutes and then stirred at 40-45° C. for 12 hours. Then the mixture was cooled to 25-30° C. within 2 hours and stirred at this temperature for one hour. The solid was filtered and rinsed with acetone (28 mL). The wet material was dried in air at 55° C. for 15 hours to afford Compound 2 (13.3 g) in 88% yield. ¹H NMR (400 MHz, DMSO-d₆) δ 8.42 (brd, 1H), 7.96 (s, 1H), 7.38 - 7.34 (m, 2H), 7.22 - 7.17 (m, 3H), 6.68 (brd, 2H), 6.09 - 5.99 (m, 2H), 5.81 (brd, 1H), 4.82 - 4.79 (m, 1H), 4.43 - 4.28 (m, 3H), 4.11 - 4.07 (m, 1H), 3.80 - 3.77 (m, 1H), 2.90 (s, 3H), 1.22 - 1.09 (m, 12H).

### Example 12. Large-Scale Manufacture of Compound 1, Compound 1-A, and Compound 2

### Part A: Synthesis of Compound 2

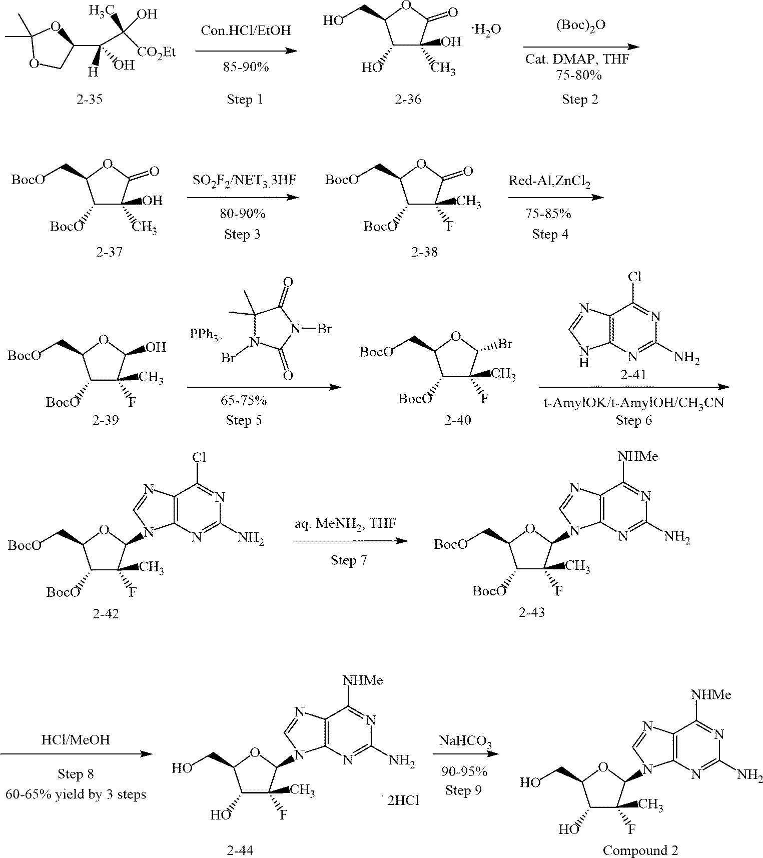

Step 1: Preparation of Compound 2-36: To a 50 L four-necked double glass flask equipped with mechanical stirrer, addition funnel, condenser and thermometer, ethanol (24 Kg) was charged. Compound 2-35 (6 Kg, 24.17 mol., 1 eq) was charged in one portion at 10° C. to afford a suspension. The mixture was warmed to 25° C. for 20 minutes to afford a clear solution. Hydrochloride acid (36%, 6.1 Kg, 60.42 mol., 2.5 eq) was charged dropwise in 2 hours while the internal temperature reached to 32° C. from 25° C. to afford a light-yellow clear solution. The reaction mixture was heated to 79±2° C. for 16 hours while maintaining the gentle reflux at which point TLC (DCM:MeOH=10:1, 1% aqueous KMnO₄ as the chromogenic reagent) showed the Compound 2-30 was consumed completely. The mixture was cooled to 40-45° C. The reaction mixture was transferred to a rotary evaporator (50 L) and then concentrated under vacuum (less than 0.09 MPa) while maintaining at 60±5° C. (water bath) until no fraction flowed out through condensing apparatus to afford a brown rope liquid. Anhydrous ethanol (12 Kg) was charged and the evaporation was repeated. Additional anhydrous ethanol (12 Kg) was charged and the evaporation was repeated once more. The crude product (oil) was dissolved in ethyl acetate (12 Kg) at 50±5° C. to afford a clear solution (no acetylation impurities were found) and transferred to the 50 L reactor. Water (0.625 Kg, 34.72 mol., 1.5 eq) was added dropwise maintaining the internal temperature between 25±5° C. during 1 hour. After another 0.5 hour with stirring, solid precipitated. The resulting suspension was stirred at 15±5° C. for 16 hours. The solid was filtered and washed with ethyl acetate (3 Kg). The wet cake was dried at 45±5° C. for 16 hours in air oven without vacuum. Compound 2-36 (3.821 Kg) was obtained in a yield of 87.8% as a white solid. ¹HNMR (DMSO-d₆): δ 5.82-5.75 (m, 2H), 5.11-5.08 (bs, 1H), 4.02-3.95 (m, 2H), 3.74-3.71 (m, 1H), 3.53-3.48 (m, 3H), 1.19 (s, 3H).

Step 2: Preparation of Compound 2-37: To a 50 L four-necked double glass reactor equipped with mechanical stirrer, addition funnel, condenser and thermometer, toluene (37.5 Kg) was charged. Compound 2-36 (7.5 Kg, 41.63 mol., 1 eq.) was charged one portion into the reactor. The mixture was heated to remove the crystal water by azeotropic distillation. When the temperature raised to 86° C., the water began to be separated out. After 8 hours, the azeotropic distillation was finished, and 700 g of water was collected. The reaction mixture was cooled to 40±5° C. and transferred to a rotary evaporator (50 L) by two batches. Then, the solution was concentrated under vacuum (less than 0.09 MPa) at 50±5° C. to give light brown liquid. Tetrahydrofuran (7.5 Kg) was charged and the mixture was concentrated under vacuum (less than 0.09 MPa) at 50±5° C. to give light brown liquid. Tetrahydrofuran (7.5 Kg) was charged again and the mixture was concentrated under vacuum (less than 0.09 MPa) at 50±5° C. until no fraction flowed out through condensing apparatus to give light brown oil. The oil was dissolved in tetrahydrofuran (30 Kg) at 50±5° C. to give a clear solution, which was then transferred to a 50 L four-necked double glass reactor. DMAP (0.51 Kg, 4.16 mol., 2.0 eq.) was charged into the reactor and the mixture was cooled to 2° C. Then a solution of (Boc)₂O (18.17 Kg, 83.26 mol., 2.0 eq.) in tetrahydrofuran (9 Kg) was added into the reaction at 0~10° C. during a period of 8 hours. Then the reaction was stirred for another 3 hours at this temperature at which point the TLC (PE: EA =5:1 to 1:1, aqueous KMnO₄ (1%) as the chromogenic reagent) showed complete consumption of Compound 2-36. The reaction mixture was transferred to a rotary evaporator (50 L) by four batches, and then concentrated under vacuum (less than 0.09 MPa) and at 50±5° C. to until no fraction flowed out through condensing apparatus to give light yellow oil. In two batches, the oil was transferred to the 50 L reactor. Water (28 Kg) was added to the above oil with stirring and maintaining the internal temperature between 15±5° C. during 2 hours. White solid precipitated during the addition of water. The resulting suspension was stirred at 15±5° C. for 16 hours. The solid was filtered to give two batches of crude product. The two batches of wet cake were combined and dissolved into dichloromethane (11.25 Kg) with stirring. After standing for 30 minutes, the phases were separated. The upper aqueous phase was discarded. The quantity of the separated water was 2.8 Kg. Then the separated organic phase was transferred to a rotary evaporator (50 L) and concentrated under vacuum (less than 0.07 MPa) at 45±5° C. to half of volume. The suspension was transferred the 50 L reactor. Heptane (22.5 Kg) was charged with stirring. The mixture was slurried at 15-20° C. for 16 hours. The solid was filtered and the cake was washed with heptane (5 Kg). The wet cake was dried at 45±5° C. for 16 hours in air oven. Compound 2-37 (11.285 Kg) was obtained in a yield of 74.8% as a white solid. ¹HNMR (CDCl₃): δ 5.10-5.08 (m, 1H), 4.51-4.45 (m, 2H), 4.31-4.27 (m, 1H), 3.44-3.43 (bs, 1H), 1.53-1.46 (m, 21H).

Step 3: Preparation of Compound 2-38: To a 50 L four-necked double glass flask equipped with mechanical stirrer, addition funnel, condenser and thermometer, DCM (12 Kg) was charged at 12° C. Et₃N-3HF (7.2 Kg, 44.7 mol., 1.5 eq.) was charged in one portion into the reactor to give a clear and colorless solution. The temperature was not changed. Then the mixture was cooled to 0° C. DBU (13.6 Kg, 89.4 mol., 3.0 eq.) was charged into the solution during a period of 2 hours while maintaining the temperature at 0~10° C. by jacket cooling. The mixture was firstly degassed under vacuum with less than 0.09 MPa. Then, SO₂F₂ (4 Kg, 39.1 mol., 1.3 eq.) was purged while a solution of Compound 2-37 (10.8 Kg, 29.8 mol., 1.0 eq.) in DCM (10.8 Kg) was dropped into the reactor simultaneously at 0~10° C. during a period of 2 hours. The color of reaction mixture became dark brown. After the addition of Compound 2-37, the SO₂F₂ was purged for another 2 hours. Totally, 4 Kg of SO₂F₂ was bubbled and then the reaction was stirred for another 16 hours at 15±5° C. at which point TLC (PE: EA =3:1, aqueous KMnO₄ (1%) as the chromogenic reagent) showed complete consumption of Compound 2-37. Then reaction mixture was transferred to a rotary evaporator (50 L) in two batches. Both batches were separately carried forward. First, the batches were concentrated under vacuum (less than 0.07 MPa) at 20±5° C. to half of volume. Water (15 Kg) was added into 50 L four-necked double glass flask, and the reaction was quenched into water at 0-5° C. within 15 minutes with stirring. The mixture was then stirred for another 10 minutes. After standing for 30 minutes, the phases were separated. The upper aqueous phase was discarded. The separated organic phase was washed with water (15 Kg) with stirring for about 10 minutes. After standing for 30 minutes, the phases were separated. The upper aqueous phase was discarded. The separated organic phase was again washed with water (15 Kg) with stirring for about 10 minutes. After standing for 30 minutes, the phases were separated. The upper aqueous phase was discarded. The separated organic phase was transferred to a rotary evaporator (50 L) and concentrated under vacuum (less than 0.07 MPa) at 20±5° C. to half of volume. Isopropanol (21.6 Kg) was charged. Then the mixture was concentrated under vacuum (less than 0.07 MPa) at 45±5° C. to half of volume. The material began to precipitate during the concentration. The first batch and second batch of material was obtained. The two batches of material were combined and transferred to the 50 L reactor. Isopropanol (21.6 Kg) was charged with stirring during 1 hour. The mixture was stirred at 0-5oC for 16 hours. The solid was filtered and the cake was washed with isopropanol (8 Kg). The wet cake was dried at 45±5° C. for 16 hours in air oven to give Compound 2-38 (9.2 Kg) with a yield of 85%. ¹HNMR (CDCl₃): δ 5.04-4.99 (m, 1H), 4.73-4.70 (m, 1H), 4.87-4.45 (m, 1H), 4.33-4.28 (m, 1H), 1.78-1.72 (m, 3H), 1.53-1.50 (m, 18H).

Step 4: Preparation of Compound 2-39: A 50 L four-necked glass reactor equipped with mechanical stirrer, addition funnel and thermometer. Anhydrous THF (26.65 Kg) was charged into the reactor. Anhydrous ZnCl₂ (1.68 Kg, 12.351 mol., 1.5 eq.) was charged into the reactor at temperature to 25±5° C. give a cloudy solution, which was stirred at room temperature for 0.5 hours. Compound 2-38 (3 Kg, 8.234 mol. 1.0 eq.) was charged into the reactor at temperature 25±5° C. to give a cloudy solution and stirred at room temperature about 0.5 hours. Red-Al (3.57 Kg, 12.351 mol., 1.5 eq.) was charged at -20 ~ -30° C. in 3 hours at the atmosphere of nitrogen and the mixture was stirred for about 30 minutes at which point the TLC (PE: EA =10:1, aqueous KMnO₄ (1%) as the chromogenic reagent) showed complete consumption of the starting material. Acetic acid (3 Kg, 49.404 mol., 6.0 eq.) was added into the reaction solution, while keeping the reaction solution temperature below -10° C. Then the reaction solution was poured into a mixture of toluene (27 Kg) and cold water (24 Kg). Hydrochloride acid (10%, 21 Kg, 1.0 mol., 7.3 eq.) was added to adjust the pH=2~3, while keeping the reaction solution temperature below -5° C. After stirring for 5 minutes and layers were separated. The water phase was extracted with toluene (13.5 Kg) again. The combined organic phases were washed with water (13.5 Kg). The separated organic phase was concentrated under vacuum less than 0.09 MPa to remove most of the THF, while keeping the temperature below 35° C. Toluene (27 Kg) was added to give a clear solution and the solution was washed with water (13.5 Kg). The organic phases were washed with aqueous NaHCO₃ (5%, 13.5 Kg). The organic phase was washed with water (13.5 Kg). Charcoal (300 mg) was added to the separated organic phase. After stirring for 1 hours at 20-30° C., the mixture was filtered, and the cake was washed with toluene (5 Kg). The combined filtrate was concentrated under vacuum less than 0.09 MPa at 40-60° C. to give crude product Compound 2-39 as an oil. Methanol (4.2 Kg) was added to give a clear solution at 40° C. Then the solution was cooled to 20-30° C. and added into water (18 Kg) slowly in 2 hours at 10-20° C. A seed of Compound 2-39 (0.02 Kg) was added. After stirring at 15-20° C. for 18 hours, the precipitated solid was filtered to give off-white solid. The solid was dried at 45° C. for 22-24 hours without vacuum to give 2706 g of Compound 2-39 as a white solid (Yield: 90%) (α/β=3:97 by ¹HNMR determination in d₆-DMSO). ¹HNMR (DMSO-d₆): δ 7.19 (d, 1H), 5.08 (m, 1H), 4.89 (m, 1H), 4.26 (m, 1H), 4.07 (m, 2H), 1.43 (m, 18H), 1.34 (m, 3H).

Step 5: Preparation of Compound 2-40: To a 50 L four-necked glass reactor equipped with mechanical stirrer, addition funnel and thermometer, acetonitrile (10.67 Kg) was charged into the reactor at -5±5° C. Triphenylphosphine (4.75 Kg, 18.45 mol., 2.5 eq.) was charged in one portion and the suspension was cooled and stirred at -5±5° C. for 30 minutes. Compound 2-39 (2.7 Kg, 7.37 mol., 1.0 eq.) was added in one portion to the suspension at this temperature. Dibromohydantoin (2.75 Kg, 9.59 mol., 1.3 eq.) was added in 20 portions to the resulting mixture in 90 minutes at the internal temperature between -5±5° C. (the suspension became to a clear solution when half of dibromohydantoin was added. The reaction solution turned to slight red after complete addition). The reaction mixture was stirred for another hour at -5±5° C. at which point the TLC (PE: EA =10:1, aqueous KMnO₄ (1%) as the chromogenic reagent) showed the reaction was complete. Ratio of α/β in the reaction mixture was 86.6/13.4 based on TLC. A solution of sodiumsulfite (0.56 Kg, 4.44 mol., 0.6 eq.), water (10 Kg), and sodiumbicarbonate (1.24 Kg, 14.76 mol., 2.0 eq.) was added dropwise to the reaction mixture via addition funnel at 0±5° C. Some of the inorganic salt precipitated. The upper organic phase was isolated and the aqueous phase and precipitated inorganic salt was discarded (ratio of α/β in the reaction mixture is 86.6:13.4 by HPLC). Ethanol (10.65 Kg) was added to the organic phase at 0±5° C. to give a clear solution. Then water (20 Kg) was added dropwise to the solution via addition funnel in a period of 30 minutes and solid precipitated. The mixture was stirred for another 0.5 hour after addition of water at 0±5° C. The solid was filtered and 3.1 Kg of wet white solid was obtained. Ethanol (8.5 Kg) was added to the reaction kettle to perform a slurry at 0±5° C. for 0.5 hours. The solid was filtered and dried at 45° C. without vacuum for 16 hours. 2388 g of white solid was obtained after drying with 97.3% purity and 76% yield. (2388 g of white solid was obtained after drying with 97.3% purity and 76% yield (HPLC showed 1.8% of β-isomer was remained).

Step 6: Preparation of Compound 2-42: Compound 2-41 (52.1 g, 308.25 mmol., 3.3 eq.) was charged into a 2.0L of four necked flask with mechanical stirrer, thermometer, condenser and drying tube. Tert-amyl alcohol (323.6 g) was added to the flask and the mechanical stirrer was turned on. Potassium tert-pentylate (35.4 g, 280.42 mmol., 3.0 eq.) was added in one portion to the suspension. The mixture was heated to 50±5° C. in 30 minutes. Then the mixture was stirred for 1 hour at 50±5° C. The reaction became a clear solution after heating for about 20 minutes and then solid precipitated. Acetonitrile (474 g) was added in one portion. Compound 2-40 (40 g, 93.44 mmol., 1.0 eq.) was added in one portion and maintained at 50±5° C. for 12-16 hours. TLC showed the reaction was complete. The reaction was cooled to 0±5° C. Hydrochloric acid (36%, 18.864 g) was added dropwise to the reaction to adjust pH=5-6 at 5±5° C. The mixture was stirred for another 30 minutes. The suspension was filtered through the pad of Celite. The filter cake was washed with acetonitrile (63.2 g) twice and the filtrate was combined. The filtrate was concentrated to about 20-30 ml of solvent in vacuum (-0.1 Mpa) at 50±5° C. Heptane (278 g) was added for further concentrate and the mixture was concentrated to about 20-30 ml of solvent at the conditions. Then a mixed solvent (EA (360.8 g): heptane (55.6 g) =1:5) were added at 30±5° C. Silica gel (200-300 mesh, 40 g) was added to the solution and stirred for 1 hour at 30±5° C. and the mixture was filtered. The filter cake was washed with mixed solvent (EA (180.4 g): heptane (27.8 g) =1:5) 3 times. The filtrate was combined and concentrated in vacuum at 50±5° C. to give a yellow foam solid (40 g).

Step 7 and 8: Preparation of Compound 2-44: Crude Compound 2-42 (40 g, 77.5 mmol., 1.0 eq.) from the last step was added to a 500 ml three-necked glass bottle with thermometer. THF (178 g) was added at 15° C. and the solution was stirred until the material was dissolved to form a homogeneous solution. Methylamine aqueous solution (calculated as 25% content, 28.9 g, 232.6 mmol., 3.0 eq.) was added in one portion and after addition, the internal temperature of the reaction system was decreased to around 5° C. The reaction system was heated to 30±5° C. and maintained at 30 ± 5° C. while stirring for about 16 hours at which point TLC indicated completion (DCM: MeOH =20:1 under UV 254). The reaction was concentrated to a small volume (about 50 ml) under vacuum (-0.1 MPa). Ethyl acetate (180 g) was added and the mixture was concentrated to a small volume (about 50 ml) by water pump under -0.1 MPa. Ethyl acetate (180 g) and water (100 g) were added and the mixture was stirred for 30 minutes. After standing for 30 minutes, the phases were separated. Water (100 g) was added into the upper organic phase and stirred for 30 minutes. After standing for 30 minutes, the phases were separated. Water (100 g) was added into the upper organic phase and stirred for 30 minutes. After standing for 30 minutes, the phases were separated. The upper organic phase was concentrated under vacuum until no liquid flew out of the condenser to obtain Compound 2-43 as a foam solid, which was directly used for the next step.

The crude Compound 2-43 was dissolved in methanol. HCl/MeOH solution (2 mol/L, 116.3 g, 232.6 mmol., 3.0 eq.) was added in one portion. The mixture was heated to reflux, and the solid precipitated after stirring for 15-20 minutes at reflux. The mixture was held at reflux for about 2 hours at which point TLC indicated completion (DCM: MeOH =20:1 under UV 254). The reaction was cooled to 15° C. and stirred for 2 hours at 15° C. The solid was filtered and washed with a small amount of methanol (9.6 g) to give crude wet product. To the wet product was added ethanol (96 g) and the mixture was heated to reflux for about 3 hours. The reaction was then cooled to 15° C. and stirred for about 2 - 3 hours. The reaction was filtered and washed with a small amount of ethanol (9.6 g) to give wet product. The wet cake was dried in air oven at 60° C. for 16 hours to give the Compound 2-44 as off-white solid (22.9 g) in 77% yield by 2 steps.

Step 9: Preparation of Compound 2: To Compound 2-44 (3.0 g, 7.79 mmol., 1.0 eq.) was added 2-methyltetrahydrofuran (25.68 g). NaHCO₃ solid (1.64 g, 1848 mmol., 2.5 eq.) was next added in one portion followed by water (3 g). The mixture was heated to 30 ± 5° C. and stirred for 30 minutes before Na₂SO₄ (3 g) was added and the reaction was stirred for 30 minutes. The mixture was filtered and the filter cake was washed with a small amount of 2-methyltetrahydrofuran (2.6 g). The phases of filtrate were separated. The upper organic phase was concentrated to dryness under reduced pressure to obtain Compound 2 as white solid in a yield of 95%.

### Part B. Synthesis of the Dihydroquinine Salt of Isopropyl (hydroxy(phenoxy)phosphoryl)-L-alaninate

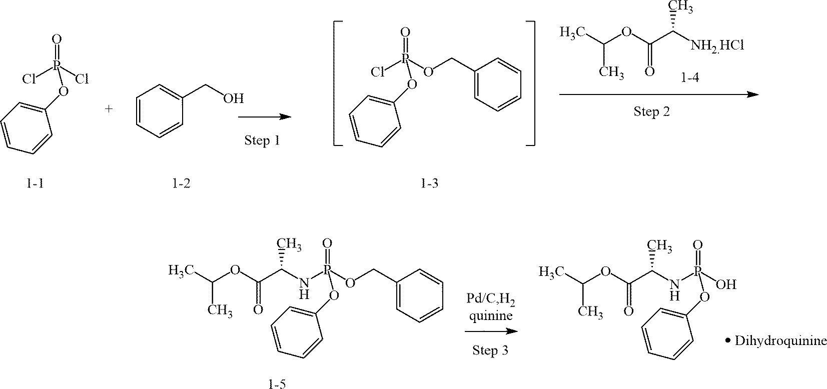

Step 1 and Step 2: A50L four-necked glass reactor equipped with mechanical stirrer, addition funnel and thermometer. Isopropyl acetate (25 Kg) was charged into the reactor and the reaction solution temperature was maintained below 10° C. Phenyl dichlorophosphate (Compound 1-1, 3.5 Kg, 16.6 mol., 1.0 eq.) was charged into the reactor give a clear solution and the solution was cooled to -10+5° C. Then a solution of benzyl alcohol (Compound 1-2, 1.89 Kg, 17.43 mol., 1.05 eq.), triethylamine (2.01 Kg, 19.9 mol., 1.2 eq.) and isopropyl acetate (3.1 Kg) were added.at -10+5° C. in 2 hours in the atmosphere of nitrogen. The mixture was stirred for 1.5 hours at -10 +5° C. at which point TLC (EtOAc:PE=1:2, UV254) showed completion. L-Alanine isopropyl ester hydrochloride (Compound 1-4, 2.78 Kg, 16.6 mol., 1.0 eq.) was charged into the reactor. Then a solution of triethylamine (3.5 Kg, 34.9 mol., 2.1 eq.) and isopropyl acetate (155 Kg) were added at -10 +5° C. about 2 hours under the protection of nitrogen. The mixture was stirred for about 1-2 hours at -10 +5° C. at which point TLC (EtOAc:PE=1:2, UV254) showed completion. The reaction mixture was filtered, and the filter cake was washed with isopropyl acetate (2.0 Kg). The filtrate was washed with water (15 Kg) below 10° C. The separated organic phase was washed with hydrochloride (1N, 15 Kg) below 10° C. The separated organic phase was washed with saturated aqueous sodium bicarbonate (15 Kg) below 10° C. The separated organic phase was washed with water (15 Kg) below 10° C. The charcoal (350 Kg) was added to the separated organic phase. Afterstirring for 2 hours at 25-30° C., the mixture was filtered, and the cake was washed with isopropyl acetate (2.0 Kg). The combined filtrate was concentrated under vacuum less than 0.09Mpa at 40-50° C. to give crude product Compound 1-5 (6.19 kg; pale yellow oil; yield: 99%mol/mol; HPLC purity: 90% by area percent).

Step 3: A 20 L three-necked glass flask equipped with mechanical stirrer and thermometer was charged into the reactor with Compound 1-5. Isopropanol (8 Kg) was charged into the reactor. Quinine (2 Kg, 6.17 mol., 1.0 eq.) was added into the reactor give a clear solution. 5% wet Pd/C (65% water by KF, 300 Kg) was charged into the reactor. Hydrogenation was performed at 20-25° C. for 48-50 hours at 1 atm of hydrogen at which point TLC showed complete consumption of quinine. The mixture was filtered through a Büchner funnel. The filtrate was concentrated to dryness under vacuum less than 0.09 Mpa at 50-60° C. to give crude product and heptane (5 Kg) was added. The mixture was concentrated under vacuum less than 0.09 Mpa at 40-45° C. and then this step was repeated again by adding fresh heptane (5 Kg). Heptane (10 Kg) was added and the mixture was stirred at 15-25° C. for 2-3 hours. The mixture was filtered to give the crude product of dihydroquinine that was then dried at 45° C. without vacuum for 16 hours to give 1.86 Kg of dihroquinine (Off-white solid; Yield: 93% mol/mol; HPLC Purity: 99.4% by area).

In an alternative Step 3, dihydroquinine is added during the hydrogenation step:

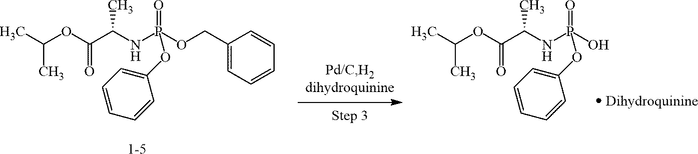

Isopropanol (175 g) was charged into a 500 ml three-necked glass flask and Compound 1-5 (35 g) and dihydroquinine (27.2 g, 0.084 mol., 1.0 eq.) were added into and give a clear solution. 5% wet Pd/C (5 g, 60% water by KF) was charged into the solution. Hydrogenation was performed at 20-25° C. for 20-24 hours at which point TLC showed completion. The mixture was filtered through a Büchner funnel. Then charcoal (2 g) was added. After stirring for 2 hours at 25-30° C., the mixture was filtered, and the cake was washed with isopropanol. The filtrate was concentrated to dryness under vacuum less than 0.09 Mpa at 50-60° C. to give crude product and methyl tert-butyl ether (100 g) was added. The mixture was concentrated under vacuum less than 0.09 Mpa at 40-45° C., and then this step was repeated again. Methyl tert-butyl ether (120 g) was added and the mixture was stirred at 50° C. for 1 hour and then stirred at 0-5° C. for 1 hour. The mixture was filtered to give crude dihydroquinine salt of isopropyl (hydroxy(phenoxy)phosphoryl)-L-alaninate. Then acetonitrile (100 g) was added and the mixture was heated to reflux to give a clear solution. Then the solution was cooled to 0-10° C. and stirred for 3 hours at this temperature. The precipitated solid was filtered, washed with cold acetonitrile (10 g), and then dried at 45° C. without vacuum for 16 hours to give 37 g of dihydroquinine salt of isopropyl (hydroxy(phenoxy)phosphoryl)-L-alaninate (Off-white solid; Yield of two steps: 65% mol/mol; HPLC Purity: 97.5% by area).

### Part C: Synthesis of Compound 1 and Compound 1-A

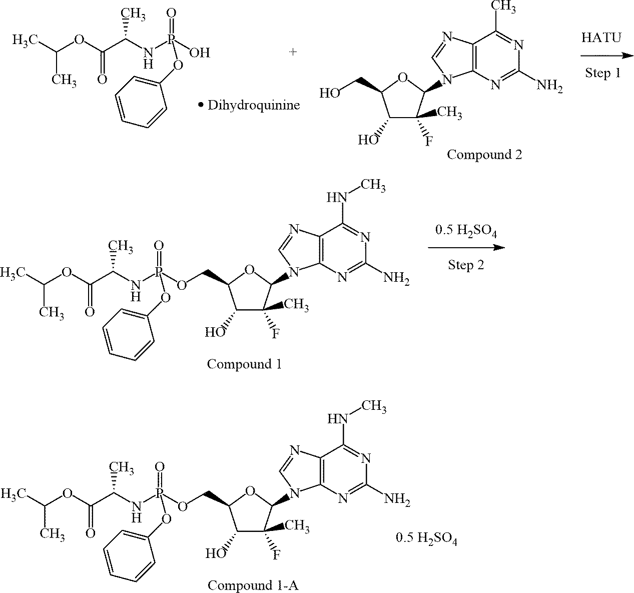

Step 1: Preparation of Compound 1: A 3 L three-necked glass flask equipped with mechanical stirrer and thermometer. Dichloromethane (2100 g) was charged into the reactor. The diydroquinine salt of isopropyl (hydroxy(phenoxy)phosphoryl)-L-alaninate (295 g, 0.480 mol., 1.5eq.), Compound 2 (100 g, 0.320 mol., 1.0 eq.), DIPEA (41.5 g, 0.320 mol., 1.0 eq.) and HATU (182.5 g, 0.480 mol., 1.5 eq.) were charged into the reactor give a cloudy solution. The mixture was heated to 40° C. and stirred for 18-20 hours at which point TLC (EtOAc:MeOH=15:1) and HPLC showed that the Compound 2 had been completely consumed. The reaction mixture was cooled to 0-10° C. Hydrochloric acid (6N, 400 g, 2.40 mol., 7.5 eq.) was slowly added at 0-10° C., and most of the dihydroquinine hydrochloride precipitated. The precipitated solid was filtered and was washed with HCl (2N, 500 g), 5% aqueous sodium bicarbonate (500 g), and water (500 g). The separated organic phase was concentrated at 40-45° C. under vacuum less than 0.09 Mpa to give yellow oil. Isopropyl acetate (360 g) and water (5000 g) were added to dissolve the above oil. The HCl (2N) was added to adjust the pH=4 to forming the salt. After standing for 10 minutes, the phases were well separated, many impurities entered the upper organic phase, and the hydrochloride of Compound 1 stayed in the down aqueous phase. Then the aqueous phase was adjusted to pH=8 with sodium bicarbonate and was washed with dichloromethane to extract Compound 1 to the organic phase. The combined organic phases were washed water and charcoal (10 g) was added. After stirring for 2 hours at 25-30° C., the mixture was filtered, and the cake was washed with dichloromethane (15 g). The dichloromethane was removed by concentration at 30-50° C. under vacuum less than 0.09 Mpa. Isopropyl acetate(180 g) was added to do the concentration again to give yellow oil product. Isopropyl acetate (540 g) was added. The mixture was heated to 50° C. to get a clear solution. After stirring at room temperature for 2 hours, the precipitation was filtered to give off-white solid of crude Compound 1. The crude Compound 1 and isopropyl acetate (540 g) were heated to around 50° C. to get a solution that was cooled to 20-25° C. and stirred for 2 hours. The precipitated solid was filtered, washed with isopropyl acetate (45 g), and dried without vacuum at 45-50° C. for 16 hours to give 112.5 g of Compound 1. (Yield: 60.5% mol/mol; HPLC purity: 99.1% by area).

Step 2: Preparation of Compound 1-A: Acetone (1000 g) was charged to a glass flask with stirring at 20-25° C. and Compound 1 (100 g, 0.172 mol.) was added to form a clear solution. Concentrated sulfuric acid (98%, 8 g, 0.082 mol., 0.475 eq.) was slowly added to the solution while the internal temperature was kept at 20-25° C. The addition time was not less than 60 minutes. The suspension was aged at an internal temperature of 20- 25° C. for 30 minutes. The suspension was heated at an internal temperature of 40-45° C. with stirring for 16 hours. The suspension was cooled to an internal temperature of 20-25° C. within 2 hours and then aged for not less than 2 hours. The solid was filtered via vacuum filtration and the wet filter cake was washed with an appropriate amount of acetone (50 g) at ambient temperature. The product was dried at 40° C. ± 5° C. for not less than 5 hours under vacuum, followed by further drying at 55° C. ± 5° C. for not less than 17 hours under vacuum and Compound 1-A was obtained in a yield of 88%.

### Example 13: Alternative Large-Scale Manufacture of Compound 1, Compound 1-A, and Compound 2

An alternative process for the preparation of Compound 1, Compound 1-A, and Compound 2 is detailed below. This process uses the crystallization of Compound 2-42 from DCM and n-heptane in step 1. This allows for better control of the impurities in the starting materials of the subsequent step. The added purity of the starting material Compound 2-42 reduces the number of side products generated in step 2, as well. With reduced side products created, the simpler mixture of products produced in step 2 can be used in step 3 without further purification. Following global Boc-deprotection, the crude reaction mixture formed after step 2 converges on the single target Compound 2. Using basic conditions to deprotect the Boc groups instead of more common acidic conditions means a subsequent step to remove the salt is not necessary, as opposed to the process in Example 12. Crystallization of the neutral amine of Compound 2 yields material ready for the coupling with the phosphoramidate.

The alternative process also prepares the dihydroquinine salt of isopropyl (hydroxy(phenoxy)phosphoryl)-L-alaninate differently. The dihydroquinine base is prepared from the hemisulfate salt of quinine in a separate reaction rather than in situ with the debenzylation of the phosphoramidate. The separated reactions provide greater control over the purity of the final dihydroquinine salt of isopropyl (hydroxy(phenoxy)phosphoryl)-L-alaninate.

It was also found that using COMU as an alternative activator to HATU provides higher diastereoselectivity at lower reaction temperatures in the coupling reaction between Compound 2 and the dihydroquinine salt of isopropyl (hydroxy(phenoxy)phosphoryl)-L-alaninate. This helps to provide a higher yield of Compound 1 while also reducing the reaction time.

### Part A: Synthesis of Compound 2

The synthesis of intermediate Compound 2-40 is performed using the same conditions described in Example 12.

### Step 1: Preparation of Compound 2-42

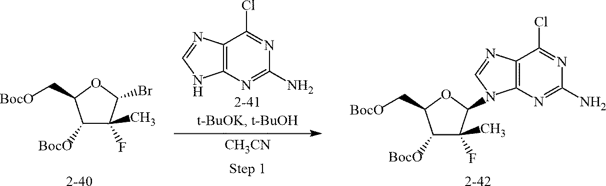

To a 5 L 3-neck glass reactor equipped with mechanical stirrer, addition funnel, and thermometer, tert-butanol (10 vol) was charged at 25° C. followed by potassium t-butoxide (3 eq). The mixture was stirred for 15 mins to give a nearly clear solution with a temperature rise to 35° C. due to the exothermic dissolution process. 2-Amino-6-chloropurine Compound 2-41 (3.1 eq) was added in 1 portion to give a suspension. The mixture was heated to 52.5° C.± 2.5° C. over a period of 30 min and stirred for 1 h at that temperature to obtain a viscous suspension. Acetonitrile (15.0 vol) was charged in 1 portion followed by Compound 2-40 (1.0 eq) in 1 portion and stirred at 52.5° C.± 2.5° C. for 16 h. Upon reaction completion, the mixture was cooled to 5° C. ± 5° C., and water (8 vol) was charged with stirring to get a clear solution, then toluene (8 vol) was added and stirred for 2-3 min. When the layers settled, the upper organic phase was separated, washed with water (8 vol), and concentrated under vacuum at 55° C. ± 5° C. The residue was further combined and concentrated under vacuum at 55° C. ± 5° C. with toluene (2 × 2 vol), then mixed with CH₂Cl₂ (5 vol) at 20° C. ± 5° C. to get a light suspension. Silica gel (200-300 mesh, 0.3 wt) was charged, stirred for 30 min, and filtered through a short silica gel pad (200-300 mesh, 0.7 wt, 8 cm diameter and 12 cm high). The wet silica pad was washed with CH₂Cl₂ (6 × 1.0 vol). The combined filtrates were concentrated under vacuum at 55° C. over a period of 50 min to give an oily solid, which was further purified by recrystallization from CH₂Cl₂ (1.5 vol) and n-heptane (8 vol). The solid was collected by filtration through a Büchner funnel and dried at 60° C. ± 5° C. in hot air for 16 h to give Compound 2-42 with 59% yield (by mol) and 99.5% a/a HPLC purity.

### Steps 2 and 3: Preparation of Compound 2-43 and Telescoping Into Compound 2

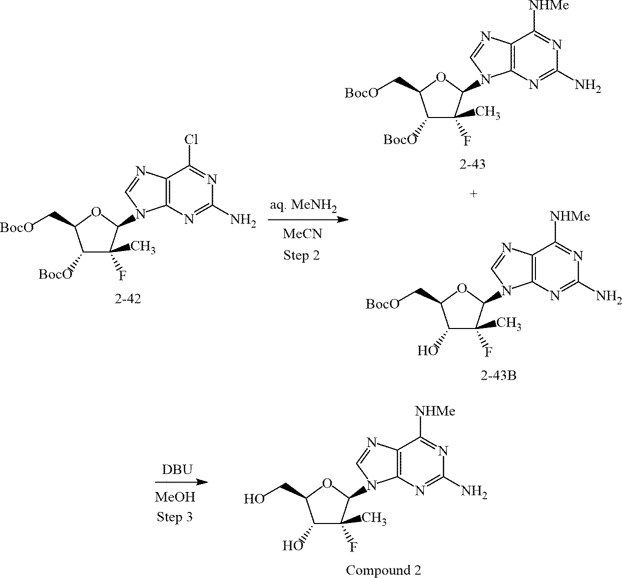

Here, the procedures of Example 12 were further developed to obtain both Compound 2-42 and Compound 2-43 as crystallizable solids, which enabled better impurity control in these steps by product isolation and purification. However, it was decided to isolate only Compound 2-42 as a crystalline intermediate but telescope Compound 2-43 obtained from Compound 2-42 to the next step without isolation. In this way, issues arising from the presence of the impurity Compound 2-43B are avoided. In addition, better conditions were established for Step 2 by using aqueous methylamine (28% w/w, 3 eq) in ACN (10 vol) at 30° C.-40° C. for the reaction and working it up with toluene. Thus, the quality of crude Compound 2-43 had greatly improved so that it could be directly used in Step 3.

To a 2 L 3-neck glass reactor equipped with mechanical stirrer, addition funnel, and thermometer, Compound 2-42 (300 g, 1.0 eq) was charged followed by acetonitrile (1500 mL, 5 vol) at 20° C. ± 5° C. and methylamine aqueous solution (calculated as 28% wt, 3.0 eq) in 1 portion at 25° C. After the addition, the internal temperature of the reaction system decreased to around 20° C. The reaction system was heated to and maintained at 35° C. ± 5° C. and stirred for about 16 h (overnight). The reaction was first monitored by TLC, which showed that the reaction was complete. HPLC showed 95.9% a/a of Compound 2-43 and 3.6% a/a of Compound 2-43B. The reaction mixture was concentrated to about 1.5 vol (450 mL) under vacuum (0.1 MPa) at 45° C.-50° C. Toluene (1500 mL, 5 vol) and water (600 mL, 2 vol) were added at 20° C. ± 5° C., and the mixture was stirred for 30 min. After standing for 30 min, the phases were separated. Water (600 mL, 2 vol) was added into the organic layer at 20° C. ± 5° C. and stirred for 30 min. After standing for 30 min, the layers were separated. This operation (i.e., water (600 mL, 2 vol) followed by layer separation) was repeated 1 more time. The upper organic phase was concentrated under vacuum to about 2 vol (600 mL). Methanol (600 mL, 2 vol) was added and concentrated under vacuum to about 2 vol. The resulting mixture was used directly in the next step.

Methanol (1500 mL, 5 vol) was added to the above Compound 2-43 solution at 20° C. ± 5° C. DBU (8.85 g, 0.1 eq) was added in 1 portion at 20° C. ± 5° C., and the mixture was heated to 60° C.-70° C. and stirred overnight at reflux. TLC showed the reaction was complete, and HPLC analysis showed 99.5% a/a of Compound 2. Acetonitrile (1800 mL, 6 vol) was added over a period of 30 min via an addition funnel, and the mixture was concentrated to about 2 vol (600 mL) at atmospheric pressure. At this time, the distillate temperature reached to 80° C. A second portion of acetonitrile (900 mL, 3 vol) was added over 10 min via the addition funnel and the mixture was concentrated to about 3 vol (900 mL) under atmospheric pressure, at which time the distillate temperature reached to 82° C. The mixture was stirred at 80° C. ± 5° C. for about 1 h, then cooled slowly to 20° C.-25° C. over a period of 2 h, with a cooling rate about 15° C. per 30 min. It was further cooled to 5° C. ± 5° C. over a period of 1 h and stirred at 5° C. ± 5° C. for 1 h. The solid was collected by filtration, and the filter cake was rinsed with acetonitrile to give a wet product. The wet cake was dried in vacuum at 55° C.-60° C. for 16 h to give Compound 2 as an off-white solid (164.9 g) in 90.8% yield with 99.8% a/a purity. The isolated solid was milled and then used in Step 7.

### Part B: Synthesis of the Dihydroquinine Salt of Isopropyl (hydroxy(phenoxy)phosphoryl)-L-alaninate

### Step 4: Preparation of Compound 1-5

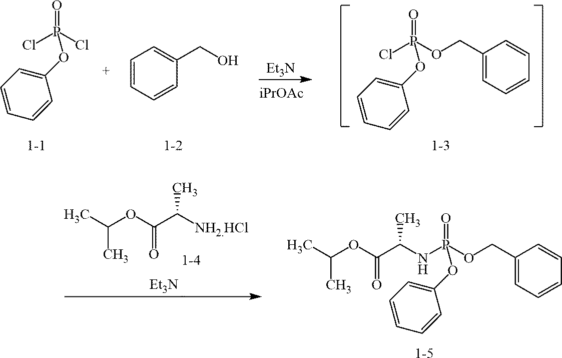

To a 50 L 4-neck glass reactor equipped with mechanical stirrer, addition funnel, and thermometer, was charged isopropyl acetate (10.3 vol). Maintaining the solution temperature below 10° C., phenyl dichlorophosphate (3.5 kg, 1.0 eq) was charged into the reactor and stirred to give a clear solution. The solution was cooled to -10° C. ± 5° C., and a solution of benzyl alcohol (1.05 eq) and triethylamine (1.2 eq) in isopropyl acetate (1.0 vol) was added over a period of 2 h under the protection of nitrogen. The mixture was stirred at -10° C. ± 5° C. for 1.5 h. When IPC showed benzyl alcohol was consumed completely, L-alanine isopropyl ester hydrochloride (1.0 eq) was charged into the reactor. To this was then charged a solution of additional triethylamine (2.1 eq) in isopropyl acetate (1.0 vol) at -10° C. ± 5° C. over a period of about 2 h under the protection of nitrogen. The stirring was continued for further 1-2 h at -10° C. ± 5° C., and the reaction progress was monitored by IPC. When the intermediate Compound 1-3 was consumed completely, the reaction mixture was filtered, and the filter cake was washed with isopropyl acetate (0.66 vol). The filtrate was cooled below 10° C. and washed successively with water (4.3 vol), hydrochloric acid aqueous solution (1 N, 4.3 vol), saturated aqueous sodium bicarbonate (4.3 vol), and water (4.3 vol) below 10° C. It was then stirred with charcoal (10% wt) for 2 h at 25° C.-30° C. and filtered. Thecake was washed with isopropyl acetate (0.66 vol), and the combined filtrate was concentrated under vacuum less than 0.09 MPa at 40° C.-50° C. to give crude product Compound 1-5 (6.19 kg) as a pale yellow oil in 99% yield with 90% a/a HPLC purity. The product was used directly in Step 6.

### Step 5: Preparation of Dihydroquinine

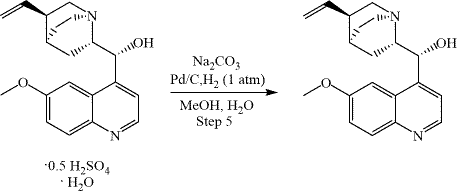

To a 1 L 3-neck glass flask equipped with mechanical stirrer, additional funnel, condenser, and thermometer, methanol (6 vol) and water (1 vol) were charged and stirred. Quinine hemisulfate monohydrate (100 g, 1 eq) and Na₂CO₃ (15 g, 0.55 eq) were charged to the reactor. The mixture was heated to 50° C.-60° C. and stirred for 4-5 h at 50° C.-60° C. Then the mixture was filtered hot and washed with methanol (0.6 vol). The filtrate was charged into a 1 L single-neck glass flask, and 5% wet Pd/C (65% water by KF, 12% wet wt) was charged. Hydrogenation was performed at 20° C.-25° C. for 10-15 h under 1 atm of hydrogen. When IPC showed the quinine was consumed completely, the mixture was filtered through a Büchner funnel, and the filter cake was rinsed with methanol (0.6 vol). The filtrate was concentrated under vacuum less than 0.09 MPa at 50° C.-60° C. to give crude product as a mixture of oil and water. The crude product was charged to 5% of sodium chloride aqueous solution (2 vol) and DCM (5 vol) and stirred for 20 mins at 20° C.-30° C. After 15 min to settle, the phases were separated, and the upper aqueous phase was extracted with additional DCM (1 vol). The organic phases were combined and concentrated under vacuum (less than 0.07 MPa) at 45° C. ± 5° C. to a quarter of the original volume then n-heptane (3 vol) was slowly charged. Then the mixture was concentrated under vacuum (less than 0.07 MPa) at 45° C. ± 5° C. to half of the volume. The material began to precipitate during the concentration, and n-Heptane (3 vol) was slowly charged and stirred for 5 h at 0° C.--10° C. The solid was collected by filtration and washed with n-heptane (1 vol) to give a wet cake, which was dried at 45° C. ± 5° C. for 16 h under vacuum to give dihydroquinine (79 g) in 95% yield 97.7% a/a HPLC purity.

### Step 6: Preparation of the Dihydroquinine Salt of Isopropyl (hydroxy(phenoxy)phosphoryl)-L-alaninate

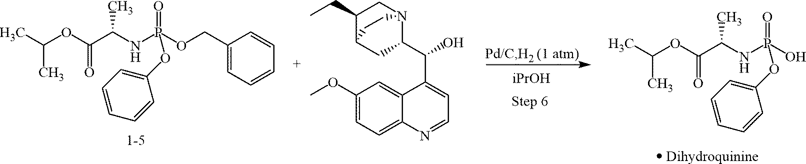

Isopropanol (356 mL, 5.5 vol) was charged into a 1000 mL 3-neck glass flask. Compound 1-5 (64 g, 1.0 eq) and dihydroquinine (50 g, 0.9 eq) were added and stirred to give a clear solution. To this solution, 5% wet Pd/C (60% water by KF, 5% w/w dry basis) was charged, and hydrogenolysis was performed at 20° C.-25° C. for 18-20 h using 1 atm of hydrogen. When the IPC-1 showed Compound 1-5 was consumed completely, the mixture was filtered through a Büchner funnel, and the filtrate was stirred with 4.0 g of charcoal for 2 hours at 25° C.-30° C. The mixture was filtered, and the cake was washed with isopropanol (50 mL, 0.8 vol). The filtrate was concentrated under vacuum less than 0.09 MPa at 50° C.-60° C. to give a crude product. Isopropyl acetate (230 mL, 3.6 vol) was added, and the mixture was concentrated again under vacuum less than 0.09 MPa at 50° C.-60° C. This step was repeated 1 more time with 3.6 vol of isopropyl acetate. To the residue, fresh isopropyl acetate (184 ml, 2.9 vol) was added, and the mixture was stirred at 80° C.-90° C. for 2-3 h to give a clear solution. Then it was cooled to 0° C.-10° C. with a cooling rate of 20° C./1 h and stirred at this temperature for 2-3 h. The solid was collected by filtration, rinsed with cold isopropyl acetate (22 mL, 0.3 vol) to give the wet cake (80 g) of crude dihydroquinine salt of isopropyl (hydroxy(phenoxy)phosphoryl)-L-alaninate, which was divided into 2 equal portions.

The first portion was dried at 50° C. without vacuum for 18 h to give 36 g of dihydroquinine salt of isopropyl (hydroxy(phenoxy)phosphoryl)-L-alaninate as an off-white solid in 76% yield with 95.85% a/a purity of the isopropyl (hydroxy(phenoxy)phosphoryl)-L-alaninate. The second portion was mixed with isopropyl acetate (80 mL) and stirred at 80° C.-90° C. for 2-3 h for complete dissolution. It was then cooled to 0° C.-10° C. with a cooling rate of 20° C./1 h and stirred for 2-3 h at this temperature. The precipitated solid was filtered, washed with cold isopropyl acetate (11 mL), and dried at 50° C. without vacuum for 18-20 h to give 33.5 g of dihydroquinine salt of isopropyl (hydroxy(phenoxy)phosphoryl)-L-alaninate as a white solid in 71% yield with 98.65% a/a purity of the isopropyl (hydroxy(phenoxy)phosphoryl)-L-alaninate.

### Part C: Synthesis of Compound 1 and 1-A

### Step 7: Preparation of Compound 1

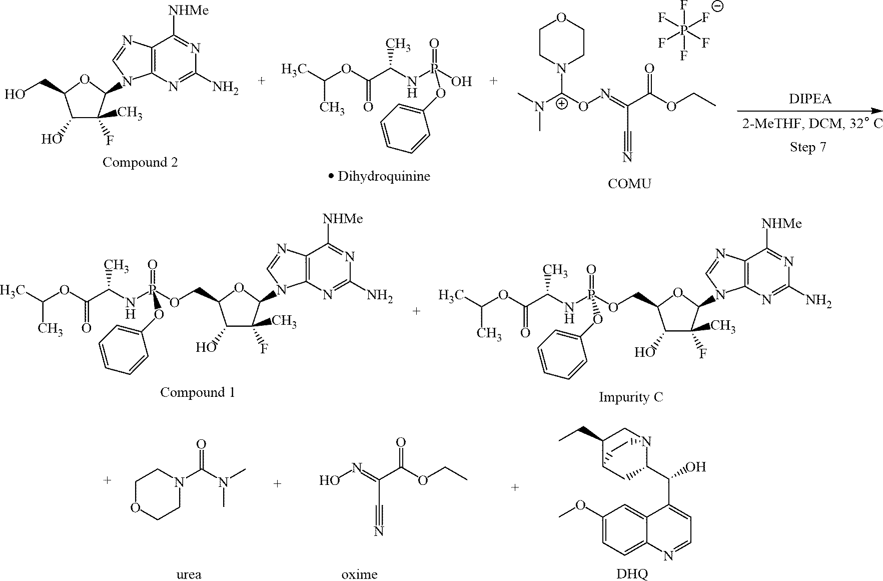

A 3 L 3-neck glass flask was equipped with a thermometer, addition funnel, and mechanical stirrer. Dichloromethane (10 vol), 2-MeTHF (20 vol), dihydroquinine salt of isopropyl (hydroxy(phenoxy)phosphoryl)-L-alaninate (1.75 eq), Compound 2 (60 g, 1.0 eq), and COMU(1.75 eq) were charged into the reactor under nitrogen atmosphere at 20° C. The resulting suspension was heated to around 30° C., and DIPEA was added slowly into the reaction mixture via the addition funnel over a period of 3 h at 30° C. ± 2.5° C. The reaction mixture was the stirred for 2 h at 30° C. ± 2.5° C. and deemed to be complete by IPC testing. The mixture was cooled to 0° C.-10° C. over a period of 1 h and washed with water (10 vol). Aq. hydrochloric acid (20% w/w, 300 g, 8.5 eq) was added slowly with stirring, over a period of 1 h, at -5° C.-0° C., when most of the dihydroquinine hydrochloride salt (DHQ.HCl) precipitated. The salt was removed by filtration, and the filtrate was washed with aq. HCl (7% w/w, 180 g, 2.0 eq) at 0° C.-10° C. to remove any residual DHQ, Compound 2, DIPEA, and urea by-products. Then it was washed with aq. 5% sodium bicarbonate (3 × 1.9 eq) to remove the oxime by-product followed by water (2 × 10 vol). Charcoal (10% w/w) was added into the organic layer, stirred for 2 h at 20° C.-30° C., filtered, and the cake was washed with dichloromethane (0.5 vol). The filtrate was then concentrated at 30° C.-55° C. under vacuum less than 0.09 MPa to around 5 vol. Toluene (600 mL, 10 vol) was added to the mixture and concentrated again at 40° C.-55° C. under vacuum less than 0.09 MPa to approx. 5 vol. Ethyl acetate (120 mL, 2 vol) and more toluene (180 mL, 3 vol) were added to the residue, and the mixture was heated to 65° C.-75° C. with stirring to give a clear solution. It was gradually cooled with stirring, first to 50° C. over a period of 1 h, then to 30° C. over a period of 1 h, and finally to 5° C. over an additional period of 1 h. The stirring was continued for further 4 h at 5° C., then the solid was collected by filtration to give crude Compound 1 as yellow solid.

The crude Compound 1 was divided into 2 equal portions. Portion 1 was further purified by 2 successive recrystallizations from ethyl acetate/toluene (1:1 v/v, 5.0 vol) and ethyl acetate (5.0 vol), whereas Portion 2 was purified by 2 successive recrystallizations from ethyl acetate (5.0 vol) each. In each case, Compound 1 was obtained with > 99.6% a/a purity and < 0.20% a/a of the isomeric impurity C as determined by HPLC. The molar yield was about 55% for Step 7.

### Step 8: Preparation of Compound 1-A

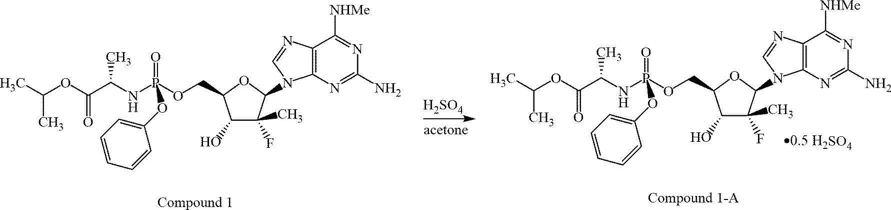

TABLE 1

Quantities of Reagents Used in Step 8

| | | | |
|---|---|---|---|
| Raw Material | Quantity | Mol. | Eq. |
| Acetone (reaction solvent) | 288 g | N/A | 12 vol |
| Compound 1 | 30 g (92% w/w assay by HPLC and titration) | 0.0475 mol | 1 eq |
| 98% H₂SO₄ | 2.25 g | 0.0225 mol | 0.475 eq |
| | 2.37 g | 0.0237 mol | 0.500 eq |
| | 2.61 g | 0.0261 mol | 0.550 eq |
| Acetone (wash) | 24 g | N/A | 1 vol |

To a 500 mL 4-neck jacketed flask equipped with a mechanical stirrer, addition funnel, condenser, and thermometer, acetone (12 vol) was charged at an external temperature of 15° C.-30° C. Compound 1 (30 g, 92% w/w assay by HPLC and titration) was charged to the reactor and stirred. The temperature of the reactor solution was controlled at 20° C.-25° C. Visual inspection confirmed reaction was a clear solution. Concentrated sulfuric acid (amounts vary as indicated in the material table above for 3 separate experiments each with 30 g of Compound 1) was slowly added to the reactor over a period of 2 h, while the internal temperature was maintained at 20° C.-25° C. The suspension was stirred at the same internal temperature of 20° C.-25° C. for 30 min, then heated 40° C.-50° C. and stirred at this temperature for 16 h. It was cooled to an internal temperature of 20° C.-25° C. over a period of 4 h and stirred for at least 2 h. The solid was collected by filtration, and the wet filter cake was washed with acetone (1 vol) at 15° C.-20° C. The product was dried at 40° C. ± 5° C. for at least 2 h in an air oven, followed by further drying at 55° C. ± 5° C. for about 18 h in the air oven. Three batches of Compound 1-A, obtained with 3 different equivalents of sulfuric acid by this procedure, were > 99.95% a/a pure by HPLC. A summary of yields and analytical data of these batches are presented in Table 2.

TABLE 2

Yields and Analytical Data for Compound 1-A from Step 8

| Attributes | Compound 1-A (0.475 eq of H₂SO₄) (Experiment 1) | Compound 1-A (0.500 eq of H₂SO₄) (Experiment 2) | Compound 1-A (0.550 eq of H₂SO₄) (Experiment 3) |
|---|---|---|---|
| Yield (molar) | 91.9% | 96.6% | 90.2% |
| Appearance | White powder with lumps | White powder with lumps | White powder with lumps |
| Related substances by HPLC | | | |
| Individual impurity, a/a | < 0.05% | < 0.05% | < 0.05% |
| Total impurities, a/a | < 0.05% | < 0.05% | < 0.05% |
| Sulfate, w/w (titration, on anhydrous basis) | 7.8% | 7.8% | 7.8% |
| Water content (KF, w/w) | 0.41% | 0.36% | 0.44% |
| Residual solvents by GC | | | |
| Acetone, w/w | 14 ppm | 13 ppm | 16 ppm |
| Bulk density | 0.31 g/mL | 0.30 g/mL | 0.23 g/mL |
| Tapped density | 0.44 g/mL | 0.42 g/mL | 0.34 g/mL |
| Particle size | DV (10) = 1.36 µm | DV (10) = 1.20 µm | DV (10) = 1.05 µm |
| | DV (50) = 22.6 µm | DV (50) = 12.0 µm | DV (50) = 4.94 µm |
| | DV (90) = 319 µm | DV (90) = 304 µm | DV (90) = 252 µm |

### Example 14: XPRD Data for Selected Compound 1-A Samples

TABLE 3

XPRDConditions and Data for Compound 1-A (Experiment 1)
SCAN: 2.0/50.0/0.02/5(deg/m), Cu(40 kV,30 mA), I(max)=536
PEAK: 47-pts/Parabolic Filter, Threshold=1.0, Cutoff=2.5%, BG=3/3.0, Peak-Top=Summit
NOTE: Intensity = Counts, 2T(0)=0.0(deg), Wavelength to Compute d-Spacing = 1.54056? (Cu/K-alpha1)

| # | 2-Theta | d(?) | BG | Height | Height% | Area | Area% | FWHM | XS(?) |
|---|---|---|---|---|---|---|---|---|---|
| 1 | 3.178 | 27.7806 | 99 | 163 | 51.1 | 5666 | 39.8 | 0.591 | 136 |
| 2 | 4.243 | 20.8064 | 140 | 122 | 38.2 | 6944 | 48.8 | 0.968 | 83 |
| 3 | 4.778 | 18.4797 | 134 | 174 | 54.5 | 7291 | 51.3 | 0.712 | 113 |
| 4 | 5.501 | 16.0518 | 125 | 71 | 22.3 | 1812 | 12.7 | 0.434 | 188 |
| 5 | 6.533 | 13.5184 | 102 | 42 | 13.2 | 191 | 1.3 | 0.073 | >1000 |
| 6 | 8.243 | 10.7173 | 106 | 92 | 28.8 | 4143 | 29.1 | 0.766 | 105 |
| 7 | 9.179 | 9.6263 | 105 | 319 | 100.0 | 12252 | 86.2 | 0.653 | 124 |
| 8 | 11.098 | 7.9662 | 108 | 40 | 12.5 | 545 | 3.8 | 0.232 | 382 |
| 9 | 11.872 | 7.4482 | 132 | 40 | 12.5 | 1387 | 9.8 | 0.589 | 137 |
| 10 | 12.512 | 7.0688 | 146 | 42 | 13.2 | 1343 | 9.4 | 0.544 | 150 |
| 11 | 13.371 | 6.6165 | 169 | 25 | 7.8 | 1023 | 7.2 | 0.696 | 116 |
| 12 | 14.798 | 5.9813 | 177 | 95 | 29.8 | 4529 | 31.8 | 0.810 | 100 |
| 13 | 17.261 | 5.1330 | 213 | 103 | 32.3 | 3506 | 24.7 | 0.579 | 141 |
| 14 | 19.600 | 4.5254 | 230 | 102 | 32.0 | 11977 | 84.2 | 1.996 | 40 |
| 15 | 20.402 | 4.3494 | 226 | 146 | 45.8 | 14179 | 99.7 | 1.651 | 49 |
| 16 | 21.314 | 4.1653 | 229 | 125 | 39.2 | 14220 | 100.0 | 1.934 | 42 |
| 17 | 24.159 | 3.6808 | 212 | 46 | 14.4 | 4032 | 28.4 | 1.402 | 58 |
| 18 | 25.294 | 3.5181 | 184 | 110 | 34.5 | 7833 | 55.1 | 1.211 | 67 |

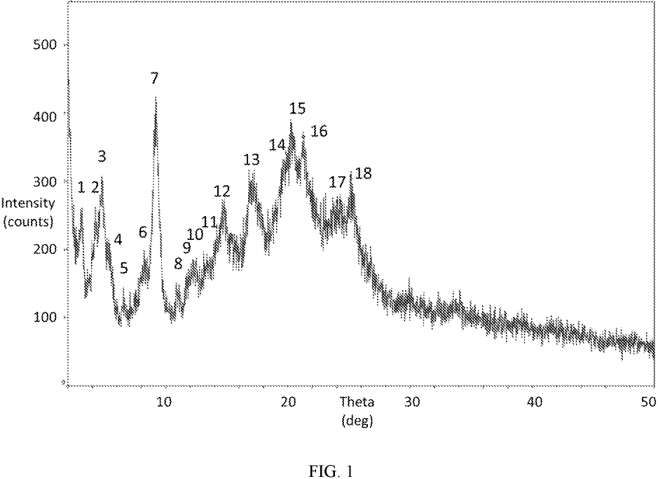

The diffractogram associated with the data from Table 3 is provided in FIG. 1

TABLE 4

XPRD Conditions and Data for Compound 1-A (Experiment 2)
SCAN: 2.0/50.0/0.02/5(deg/m), Cu(40 kV,30 mA), I(max)=460
PEAK: 47-pts/Parabolic Filter, Threshold=1.0, Cutoff=2.5%, BG=3/3.0, Peak-Top=Summit
NOTE: Intensity = Counts, 2T(0)=0.0(deg), Wavelength to Compute d-Spacing = 1.54056? (Cu/K-alpha1)

| # | 2-Theta | d(?) | BG | Height | Height% | Area | Area% | FWHM | XS(?) |
|---|---|---|---|---|---|---|---|---|---|
| 1 | 4.181 | 21.1178 | 116 | 110 | 35.5 | 2846 | 16.0 | 0.440 | 186 |
| 2 | 4.780 | 18.4703 | 107 | 131 | 42.3 | 7073 | 39.7 | 0.918 | 87 |
| 3 | 5.477 | 16.1227 | 97 | 87 | 28.1 | 2853 | 16.0 | 0.557 | 145 |
| 4 | 8.179 | 10.8008 | 95 | 81 | 26.1 | 3808 | 21.4 | 0.799 | 100 |
| 5 | 9.139 | 9.6689 | 94 | 310 | 100.0 | 13645 | 76.6 | 0.748 | 107 |
| 6 | 10.989 | 8.0448 | 100 | 54 | 17.4 | 513 | 2.9 | 0.162 | 629 |
| 7 | 12.420 | 7.1207 | 137 | 47 | 15.2 | 1432 | 8.0 | 0.518 | 157 |
| 8 | 13.382 | 6.6109 | 159 | 39 | 12.6 | 2333 | 13.1 | 1.017 | 79 |
| 9 | 13.543 | 6.5327 | 163 | 31 | 10.0 | 2370 | 13.3 | 1.300 | 62 |
| 10 | 14.762 | 5.9957 | 172 | 92 | 29.7 | 5678 | 31.9 | 1.049 | 77 |
| 11 | 16.980 | 5.2175 | 238 | 56 | 18.1 | 1065 | 6.0 | 0.304 | 279 |
| 12 | 19.659 | 4.5119 | 255 | 113 | 36.5 | 10826 | 60.8 | 1.629 | 50 |
| 13 | 20.379 | 4.3542 | 248 | 146 | 47.1 | 10714 | 60.2 | 1.248 | 65 |
| 14 | 21.245 | 4.1787 | 238 | 196 | 63.2 | 17809 | 100.0 | 1.545 | 52 |
| 15 | 22.081 | 4.0223 | 250 | 78 | 25.2 | 2844 | 16.0 | 0.620 | 132 |
| 16 | 23.867 | 3.7252 | 225 | 77 | 24.8 | 5916 | 33.2 | 1.306 | 62 |
| 17 | 24.282 | 3.6625 | 216 | 86 | 27.7 | 6744 | 37.9 | 1.333 | 61 |
| 18 | 25.318 | 3.5149 | 193 | 147 | 47.4 | 9878 | 55.5 | 1.142 | 72 |
| 19 | 33.858 | 2.6453 | 113 | 51 | 16.5 | 981 | 5.5 | 0.327 | 267 |

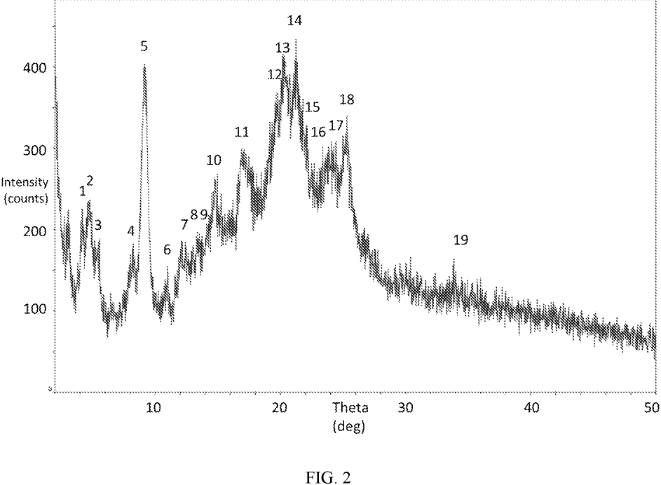

The diffractogram associated with the data from Table 4 is provided in FIG. 2

TABLE 5

XPRD Conditions and Data for Compound 1-A (Experiment 3)
SCAN: 2.0/50.0/0.02/5(deg/m), Cu(40 kV,30 mA), I(max)=532
PEAK: 45-pts/Parabolic Filter, Threshold=1.0, Cutoff=2.5%, BG=3/3.0, Peak-Top=Summit
NOTE: Intensity = Counts, 2T(0)=0.0(deg), Wavelength to Compute d-Spacing = 1.54056? (Cu/K-alpha1)

| # | 2-Theta | d(?) | BG | Height | Height% | Area | Area% | FWHM | XS(?) |
|---|---|---|---|---|---|---|---|---|---|
| 1 | 4.103 | 21.5152 | 132 | 108 | 24.9 | 3954 | 24.9 | 0.622 | 129 |
| 2 | 4.759 | 18.5543 | 120 | 200 | 46.1 | 9265 | 58.4 | 0.788 | 102 |
| 3 | 5.497 | 16.0637 | 106 | 118 | 27.2 | 3932 | 24.8 | 0.566 | 143 |
| 4 | 8.223 | 10.7435 | 102 | 108 | 24.9 | 4502 | 28.4 | 0.709 | 114 |
| 5 | 9.140 | 9.6675 | 98 | 434 | 100.0 | 15283 | 96.4 | 0.599 | 135 |
| 6 | 10.983 | 8.0487 | 106 | 52 | 12.0 | 512 | 3.2 | 0.167 | 595 |
| 7 | 12.378 | 7.1447 | 141 | 55 | 12.7 | 1536 | 9.7 | 0.475 | 172 |
| 8 | 13.169 | 6.7174 | 155 | 41 | 9.4 | 2797 | 17.6 | 1.160 | 69 |
| 9 | 13.265 | 6.6691 | 158 | 62 | 14.3 | 2757 | 17.4 | 0.756 | 107 |
| 10 | 13.356 | 6.6237 | 161 | 45 | 10.4 | 2755 | 17.4 | 1.041 | 77 |
| 11 | 14.763 | 5.9954 | 203 | 71 | 16.4 | 1366 | 8.6 | 0.327 | 257 |
| 12 | 17.019 | 5.2054 | 225 | 119 | 27.4 | 3476 | 21.9 | 0.497 | 165 |
| 13 | 17.899 | 4.9514 | 230 | 70 | 16.1 | 3133 | 19.8 | 0.761 | 107 |
| 14 | 20.441 | 4.3411 | 234 | 190 | 43.8 | 15854 | 100.0 | 1.419 | 57 |
| 15 | 21.301 | 4.1677 | 235 | 163 | 37.6 | 11758 | 74.2 | 1.226 | 66 |
| 16 | 22.115 | 4.0162 | 246 | 74 | 17.1 | 2893 | 18.2 | 0.665 | 123 |
| 17 | 23.601 | 3.7666 | 224 | 72 | 16.6 | 5994 | 37.8 | 1.415 | 57 |
| 18 | 24.318 | 3.6572 | 208 | 102 | 23.5 | 9206 | 58.1 | 1.534 | 53 |
| 19 | 25.281 | 3.5200 | 190 | 156 | 35.9 | 11297 | 71.3 | 1.231 | 66 |
| 20 | 29.596 | 3.0158 | 126 | 40 | 9.2 | 1018 | 6.4 | 0.433 | 195 |
| 21 | 29.739 | 3.0016 | 125 | 29 | 6.7 | 1042 | 6.6 | 0.611 | 136 |
| 22 | 31.196 | 2.8647 | 118 | 26 | 6.0 | 586 | 3.7 | 0.383 | 223 |
| 23 | 33.864 | 2.6449 | 106 | 40 | 9.2 | 2260 | 14.3 | 0.961 | 87 |

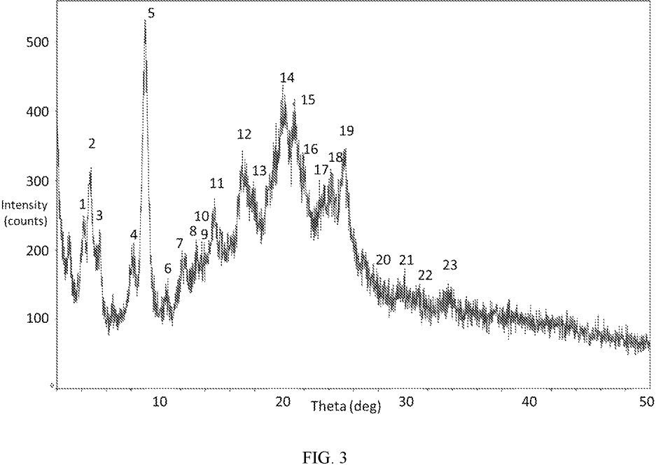

The diffractogram associated with the data from Table 5 is provided in FIG. 3

### Example 15: Differential Scanning Calorimetry Data for Selected Compound 1-A Samples

### Compound 1-A From Experiment 1

Processing software: METTLER TOLEDO STARe 15.00
Sample: 2.7560 mg of Compound 1-A from Experiment 1
Sample Holder: Aluminum Standard 40 µL; weight: 0; material: aluminum
Method: 80-200 10K N2 50; dt 1.00 s; [1] 80.0..200.0° C., 10.00 K/min, N2 50.0 ml/min; synchronization enabled

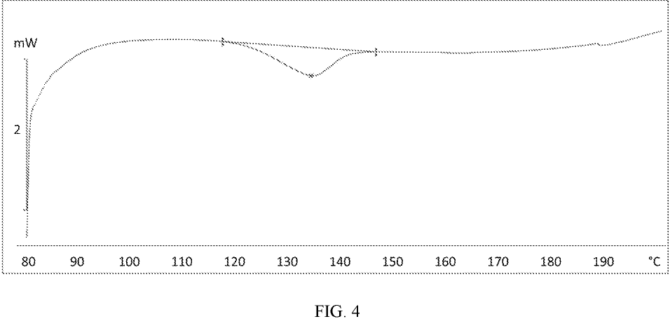

The DSC thermogram showed an endotherm peak with onset at 121.71° C., peak at 133.70° C., and endset at 141.02° C. with an integral of -26.34 mJ. The thermogram of this sample is shown in FIG. 4.

### Compound 1-A From Experiment 2

Processing software: METTLER TOLEDO STARe 15.00
Sample: 2.0660 mg of Compound 1-A from Experiment 2
Sample Holder: Aluminum Standard 40 µL; weight: 0; material: aluminum
Method: 80-200 10K N2 50; dt 1.00 s; [1] 80.0..200.0° C., 10.00 K/min, N2 50.0 ml/min; synchronization enabled

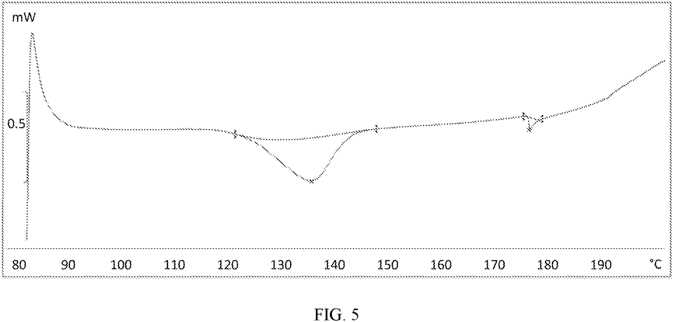

The DSC thermogram showed two endotherm peaks. The first peak with onset at 122.35° C., peak at 133.55° C., and endset at 140.41° C. with an integral of -14.97 mJ. The second peak with onset at 174.19° C., peak at 174.57° C., and endset at 176.18° C. with an integral of -0.43 mJ. The thermogram of this sample is shown in FIG. 5.

### Compound 1-A From Experiment 3

Processing software: METTLER TOLEDO STARe 15.00
Sample: 1.9360 mg of Compound 1-A from Experiment 3
Sample Holder: Aluminum Standard 40 µL; weight: 0; material: aluminum
Method: 80-200 10K N2 50; dt 1.00 s; [1] 80.0..200.0° C., 10.00 K/min, N2 50.0 ml/min; synchronization enabled

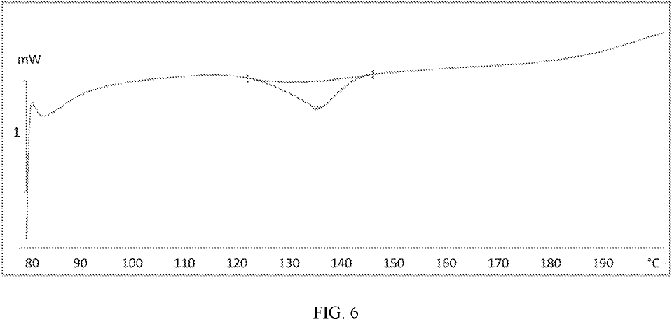

The DSC thermogram showed an endotherm peak with onset at 129.41° C., peak at 134.55° C., and endset at 141.56° C. with an integral of -13.10 mJ. The thermogram of this sample is shown in FIG. 6.

This specification has been described with reference to embodiments of the invention. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

What is claimed is:

1. A process for preparing a diastereomer Sₚ-phosphoramidate nucleotide of Formula XVI, wherein the nucleotide of Formula XVI is greater than about 90% pure, comprising the steps of (a) contacting the nucleoside Compound 2 with a compound of Formula XVII dihydroquinine salt, in the presence of a benzotriazole- or uronium-based activator and a base to afford the diastereomer Sₚ-phosphoramidate nucleotide of Formula XVI: 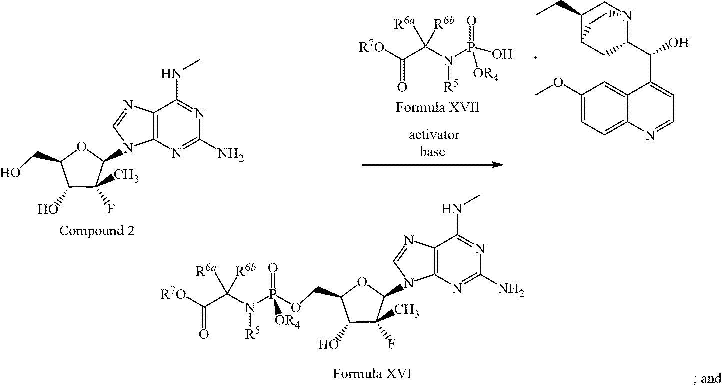
(b) optionally further purifying the diastereomerically enriched Sₚ-phosphoramidate nucleotide of Formula XVI to increase the purity;
wherein:
R⁴ is hydrogen, C₁₋₆alkyl, C₃₋₇cycloalkyl, or aryl;
R⁵ is hydrogen or C₁₋₆alkyl;
R^{6a} and R^{6b} are independently selected from the group consisting of hydrogen, C₁₋₆alkyl, and C₃₋₇cycloalkyl; and
R⁷ is hydrogen, C₁₋₆alkyl, C₁₋₆haloalkyl, or C₃₋₇cycloalkyl.

2. The process of claim 1 wherein R⁴ is aryl.

3. The process of claim 1 wherein R⁵ is hydrogen.

4. The process of claim 1 wherein at least one of R^{6a} and R^{6b} is hydrogen.

5. The process of claim 1 wherein R^{6a} and R^{6b} are hydrogen and methyl.

6. The process of claim 1 wherein R⁷ is C₁₋₆alkyl.

7. The process of claim 1 wherein R is isopropyl or ethyl.

8. The process of claim 1 wherein the uronium-based activator is COMU ((1-Cyano-2-ethoxy-2-oxoethylidenaminooxy)dimethylamino-morpholino-carbenium hexafluorophosphate).

9. The process of claim 1 wherein the benzotriazole-based activator is selected from HOBt ((1-hydroxybenzotriazole), PyBOP (benzotriazol-1-yloxytri(pyrrolidino)phosphonium hexafluorophosphate), HATU (O-(7-azabenzotriazol-1-yl)-1,1,3,3-tetramethyluronium hexafluorophosphate), HBTU (3-[bis(dimethylamino)methyliumyl]-3H-benzotriazol-1-oxide hexafluorophosphate), HCTU (2-(6-Chloro-1H-benzotriazole-1-yl)-1,1,3,3-tetramethylaminium hexafluorophosphate), and TBTU (O-benzotriazol-1-yl-1,1,3,3-tetramethyluronium tetrafluoroborate).

10. The process of claims 9 wherein the benzotriazole-based activator is HATU (O-(7-azabenzotriazol-1-yl)-1,1,3,3-tetramethyluronium hexafluorophosphate).

11. The process of claim 1, wherein the base is DIPEA (N,N-diisopropylethylamine).

12. The process of claims 1 wherein step (a) is performed in a polar aprotic solvent.

13. The process of claim 12 wherein the solvent is selected from dimethylformamide (DMF), dichloromethane (DCM), tetrahydrofuran (THF), 2-methyltetrahydrofuran (2-MeTHF), ethyl acetate (EtOAc), acetonitrile (MeCN), dimethyl sulfoxide (DMSO), acetone, and N-methylpyrrolidone.

14. The process of claim 1 wherein step (a) is performed in a mixture of solvents selected from dimethylformamide (DMF), dichloromethane (DCM), tetrahydrofuran (THF), 2-methyltetrahydrofuran (2-MeTHF), ethyl acetate (EtOAc), acetonitrile (MeCN), dimethyl sulfoxide (DMSO), acetone, and N-methylpyrrolidone.

15. The process of claim 14 wherein the mixture of solvents comprises dichloromethane (DCM) and 2-methyltetrahydrofuran (2-MeTHF).

16. The process of claim 1, wherein the base is of the formula NR₃, wherein R can be selected independently in each instance from H, alkyl, aryl, heteroaryl, alkenyl, alkynyl, benzyl and allyl, wherein at least one R is not hydrogen.

17. A process for preparing Compound 1, wherein the diastereomeric purity is greater than about 90%, comprising the steps of (a) contacting the nucleoside Compound 2 with dihydroquinine salt of isopropyl (hydroxy(phenoxy)phosphoryl)-L-alaninate, in the presence of a benzotriazole- or uronium-based activator and a base to afford the Compound 1: 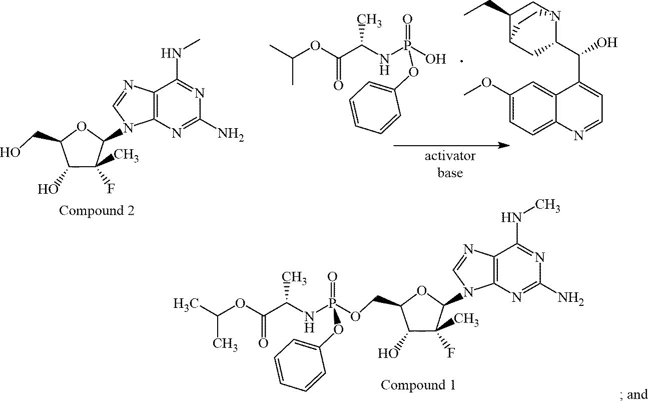
(b) optionally further purifying the diastereomerically enriched Sₚ-phosphoramidate nucleotide of Formula XVI to increase the purity.

18. The process of claim 17, wherein the base is of the formula NR₃, wherein R can be selected independently in each instance from H, alkyl, aryl, heteroaryl, alkenyl, alkynyl, benzyl and allyl, wherein at least one R is not hydrogen.

19. The process of claim 17 wherein the uronium-based activator is COMU ((1-Cyano-2-ethoxy-2-oxoethylidenaminooxy)dimethylamino-morpholino-carbenium hexafluorophosphate).

20. The process of claim 18, wherein the base is DIPEA (N,N-diisopropylethylamine).

21. The process of claim 17, wherein step (a) is performed in a mixture of solvents comprising dichloromethane and 2-MeTHF.

22. The process of claim 17, wherein the uronium-based activator is COMU ((1-Cyano-2-ethoxy-2-oxoethylidenaminooxy)dimethylamino-morpholino-carbenium hexafluorophosphate), the base is DIPEA (N,N-diisopropylethylamine), and step (a) is performed in a mixture of solvents comprising dichloromethane and 2-MeTHF.

23. The process of claim 17, wherein the benzotriazole-based activator is HATU (O-(7-azabenzotriazol-1-yl)-1,1,3,3-tetramethyluronium hexafluorophosphate).

24. The process of claim 23, wherein the base is quinine.

25. The process of claim 24, wherein step (a) is performed in a polar aprotic solvent.

26. The process of claim 25, wherein the polar aprotic solvent is dichloromethane.

27. The process of claim 17, wherein the benzotriazole-based activator is HATU (O-(7-azabenzotriazol-1-yl)-1,1,3,3-tetramethyluronium hexafluorophosphate), the base is quinine, and step (a) is performed in dichloromethane.

28. The process of claim 17, wherein the ratio of Sₚ:Rₚ diastereomers before step (b) is greater than about 60:40.

29. The process of claim 17, wherein the ratio of Sₚ:Rₚ diastereomers before step (b) is greater than about 70:30.

30. The process of claim 17, wherein the ratio of Sₚ:Rₚ diastereomers before step (b) is greater than about 80:20.

31. The process of claim 17, wherein the ratio of Sₚ:Rₚ diastereomers before step (b) is greater than about 90:10.

32. The process of claim 17, wherein the purification of step (b) is a selective crystallization.

33. The process of claim 32, wherein the crystallization is conducted in a polar organic solvent.

34. The process of claim 33, wherein the crystallization is conducted in an alkyl ester.

35. The process of claim 34, wherein the crystallization is conducted in ethyl acetate or isopropyl acetate.

36. The process of claim 32, wherein the crystallization is conducted in a mixture of solvents.

37. The process of claim 36, wherein the crystallization is conducted in a mixture of polar organic solvent and aromatic solvent.

38. The process of claim 37, wherein the crystallization is conducted in a mixture of ethyl acetate and toluene.

39. The process of claim 17, further comprising step (c) wherein Compound 1 is converted to a pharmaceutically acceptable salt.

40. The process of claim 39, wherein the pharmaceutically acceptable salt is the hemi-sulfate salt.

41. A compound of Formula XVII: 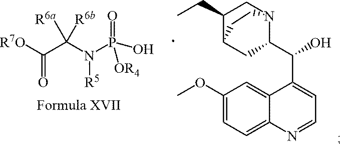
wherein:
R⁴ is hydrogen, C₁₋₆alkyl, C₃₋₇cycloalkyl, or aryl;
R⁵ is hydrogen or C₁₋₆alkyl;
R^{6a} and R^{6b} are independently selected from hydrogen, C₁₋₆alkyl, or C₃₋₇cycloalkyl; and
R⁷ is hydrogen, C₁₋₆alkyl, C₁₋₆haloalkyl, or C₃₋₇cycloalkyl.

42. The compound of claim 41, wherein R⁴ is aryl.

43. The compound of claim 42, wherein R⁴ is phenyl.

44. The compound of claim 41, wherein R⁵ is hydrogen.

45. The compound of claim 41, wherein at least one of R^{6a} and R^{6b} is hydrogen.

46. The compound of claim 41, wherein R^{6a} and R^{6b} are hydrogen and methyl.

47. The compound of claim 41, wherein R⁷ is C₁₋₆alkyl.

48. The compound of claim 41, wherein R⁷ is isopropyl.

49. The compound of claim 41, of the structure: 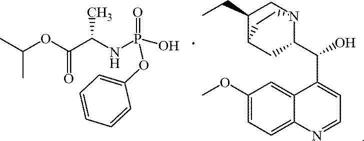
.
